(19) United States
(12) Patent Application Publication
(10) Pub. No.: US 2023/0157660 A1
(43) Pub. Date: May 25, 2023

(54) IMAGING SYSTEMS AND METHODS

(71) Applicant: SHANGHAI UNITED IMAGING HEALTHCARE CO., LTD., Shanghai (CN)

(72) Inventors: Jiali TU, Shanghai (CN); Wei LI, Shanghai (CN); Yifeng ZHOU, Shanghai (CN); Xingyue YI, Shanghai (CN)

(73) Assignee: SHANGHAI UNITED IMAGING HEALTHCARE CO., LTD., Shanghai (CN)

(21) Appl. No.: 18/157,791

(22) Filed: Jan. 20, 2023

Related U.S. Application Data
(63) Continuation of application No. PCT/CN2020/104970, filed on Jul. 27, 2020.

(51) Int. Cl. A61B 6/00; A61B 6/03

(52) U.S. Cl. CPC A61B 6/547; A61B 6/032; G06T 2207/30004

(57) ABSTRACT

The present disclosure provides systems and methods for performing an automated scan preparation for a scan of a target subject. The automated scan preparation may include, for example, identifying a target subject to be scanned, generating a target posture model of the target subject, causing a movable component of a medical imaging device to move to its target position, controlling a light field of the medical imaging device, determining a target subject orientation, determining a dose estimation, selecting at least one target ionization chamber, determining whether the posture of the target subject needs to be adjusted, determining one or more scanning parameters (e.g., a size of a light field), performing a preparation check, or the like, or any combination thereof.

### CROSS-REFERENCE TO RELATED APPLICATIONS

This application is a Continuation of International Application No. PCT/CN2020/104970 filed on Jul. 27, 2020, the contents of which are hereby incorporated by reference.

### TECHNICAL FIELD

The present disclosure generally relates to medical imaging, and more particularly, relates to systems and methods for automated scan preparation in medical imaging.

### BACKGROUND

Medical imaging technique is widely used in clinical examinations and medical diagnoses in recent years. For example, with the development of X-ray imaging technology, a digital radiography (DR) system has become more and more important in, such as, breast tomosynthesis, chest examination, or the like.

### SUMMARY

According to a first aspect of the present disclosure, a method for subject identification may include one or more operations. The one or more operations may be implemented on a computing device having one or more processors and one or more storage devices. The one or more processors may obtain image data of at least one candidate subject. The image data may be captured by an image capturing device when or after the at least one candidate subject enters an examination room. The one or more processors may also obtain reference information associated with a target subject to be examined. The one or more processors may further identify, from the at least one candidate subject, the target subject based on the reference information and the image data.

According to a second aspect of the present disclosure, a method for generating a target posture model of a target subject may include one or more operations. The one or more operations may be implemented on a computing device having one or more processors and one or more storage devices. The one or more processors may obtain image data of a target subject. The one or more processors may generate a subject model of the target subject based on the image data. The one or more processors may also obtain a reference posture model associated with the target subject. The one or more processors may further generate the target posture model of the target subject based on the subject model and the reference posture model.

According to a third aspect of the present disclosure, a method for scan preparation may include one or more operations. The one or more operations may be implemented on a computing device having one or more processors and one or more storage devices. The one or more processors may obtain image data of a target subject. For one or more movable components of a medical imaging device, the one or more processors may determine, based on the image data, a target position of each of the one or more movable components. For each of the one or more movable components of the medical imaging device, the one or more processors may also cause the movable component to move to the target position of the movable component. The one or more operations may further cause the medical imaging device to scan the target subject when the each of the one or more movable components of the medical imaging device is at its respective target position.

According to a fourth aspect of the present disclosure, a method for controlling a light field of a medical imaging device may include one or more operations. The one or more operations may be implemented on a computing device having one or more processors and one or more storage devices. The one or more processors may obtain image data of a target subject to be scanned by the medical imaging device. The image data may be captured by an imaging capture device. The one or more processors may also determine, based on the image data, one or more parameter values of the light field. The one or more processors may further cause the medical imaging device to scan the target subject according to the one or more parameter values of the light field.

According to a fifth aspect of the present disclosure, a method for determining a target subject orientation may include one or more operations. The one or more operations may be implemented on a computing device having one or more processors and one or more storage devices. The one or more processors may obtain a first image of a target subject. The one or more processors may also determine, based on the first image, an orientation of the target subject. The one or more processors may further cause a terminal device to display a second image of the target subject based on the first image and the orientation of the target subject, wherein a representation of the target subject having a reference orientation in the second image.

According to a sixth aspect of the present disclosure, a method for dose estimation may include one or more operations. The one or more operations may be implemented on a computing device having one or more processors and one or more storage devices. The one or more processors may obtain at least one parameter value of at least one scanning parameter relating to a scan to be performed on a target subject. The one or more processors may also obtain a relationship between a reference dose and the at least one scanning parameter. The one or more processors may further determine, based on the relationship and the at least one parameter value of the at least one scanning parameter, a value of an estimated dose associated with the target subject.

According to a seventh aspect of the present disclosure, an imaging method may include one or more operations. The one or more operations may be implemented on a computing device having one or more processors and one or more storage devices. The one or more processors may obtain target image data of a target subject to be scanned by a medical imaging device. The medical imaging device may include a plurality of ionization chambers. The one or more processors may also select, among the plurality of ionization chambers, at least one target ionization chamber based on the target image data. The one or more processors may further cause the medical imaging device to scan the target subject using the at least one target ionization chamber.

According to an eighth aspect of the present disclosure, a method for subject positioning may include one or more operations. The one or more operations may be implemented on a computing device having one or more processors and one or more storage devices. The one or more processors may obtain target image data of a target subject holding a posture captured by an image capturing device. The one or more processors may also obtain a target posture model representing a target posture of the target subject. The one or more processors may further determine, based on the target image data and the target posture model, whether the posture of the target subject needs to be adjusted.

According to a ninth aspect of the present disclosure, a method for image display may include one or more operations. The one or more operations may be implemented on a computing device having one or more processors and one or more storage devices. The one or more processors may obtain image data of a target subject scanned or to be scanned by a medical imaging device. The one or more processors may also generate a display image based on the image data. The one or more processors may further transmit the display image to a terminal device for display.

According to a tenth aspect of the present disclosure, an imaging method may include one or more operations. The one or more operations may be implemented on a computing device having one or more processors and one or more storage devices. The one or more processors may cause a supporting device to move a target subject from an initial subject position to a target subject position. The one or more processors may further cause a medical imaging device to perform a scan on a region of interest (ROI) of a target subject holding an upright posture. The target subject may be supported by the supporting device at the target subject position during the scan. The one or more processors may obtain scan data relating to the scan. The one or more processors may further generate an image corresponding to the ROI based on the scan data.

According to an eleventh aspect of the present disclosure, a method for determining a target subject orientation may include one or more operations. The one or more operations may be implemented on a computing device having one or more processors and one or more storage devices. The one or more processors may obtain an image of a target subject. The one or more processors may determine, based on the image, an orientation of the target subject. The one or more processors may adjust the image based on the orientation of the target subject. The one or more processors may cause a terminal device to display an adjusted image of the target subject. A representation of the target subject may have a reference orientation in the adjusted image.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device according to some embodiments of the present disclosure;

FIG. 4A is a schematic diagram illustrating an exemplary medical imaging device according to some embodiments of the present disclosure;

FIG. 4B is a schematic diagram illustrating an exemplary supporting device of a medical imaging device according to some embodiments of the present disclosure;

FIG. 5A is a flowchart illustrating a traditional process for scanning a target subject;

FIG. 5B is a flowchart illustrating an exemplary process for scanning a target subject according to some embodiments of the present disclosure;

FIG. 6 is a flowchart illustrating an exemplary process for scan preparation according to some embodiments of the present disclosure;

FIG. 7 is a block diagram illustrating an exemplary processing device according to some embodiments of the present disclosure;

FIG. 8 is a flowchart illustrating an exemplary process for identfiying a target subject to be scanned according to some embodiments of the present disclosure;

FIG. 9 is a flowchart illustrating an exemplary process for generating a target posture model of a target subject according to some embodiments of the present disclosure;

FIG. 10 is a flowchart illustrating an exemplary process for scan preparation according to some embodiments of the present disclosure;

FIG. 11A is a schematic diagram illustrating an exemplary patient model of a patient according to some embodiments of the present disclosure;

FIG. 11B is a schematic diagram illustrating an exemplary patient model of a patient according to some embodiments of the present disclosure;

FIG. 12 is a flowchart illustrating an exemplary process for controlling a light field of a medical imaging device according to some embodiments of the present disclosure;

FIG. 13 is a flowchart illustrating an exemplary process for determining an orientation of a target subject according to some embodiments of the present disclosure;

FIG. 14 is a schematic diagram illustrating exemplary images of a hand of different orientations according to some embodiments of the present disclosure;

FIG. 15 is a flowchart illustrating an exemplary process for dose estimation according to some embodiments of the present disclosure;

FIG. 16A is a flowchart illustrating an exemplary process for selecting a target ionization chamber among a plurality of ionization chambers according to some embodiments of the present disclosure;

FIG. 16B is a flowchart illustrating an exemplary process for selecting at least one target ionization chamber for an ROI of a target subject based on target image data of the target subject according to some embodiments of the present disclosure;

FIG. 16C is a flowchart illustrating an exemplary process for selecting at least one target ionization chamber for an ROI of a target subject based on target image data of the target subject according to some embodiments of the present disclosure;

FIG. 17 is a flowchart illustrating an exemplary process for subject positioning according to some embodiments of the present disclosure;

FIG. 18 is a schematic diagram illustrating an exemplary composite image according to some embodiments of the present disclosure;

FIG. 19 is a flowchart illustrating an exemplary process for image display according to some embodiments of the present disclosure;

FIG. 20 is a schematic diagram of an exemplary display image relating to a target subject according to some embodiments of the present disclosure; and

FIG. 21 is a flowchart illustrating an exemplary process for imaging a target subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

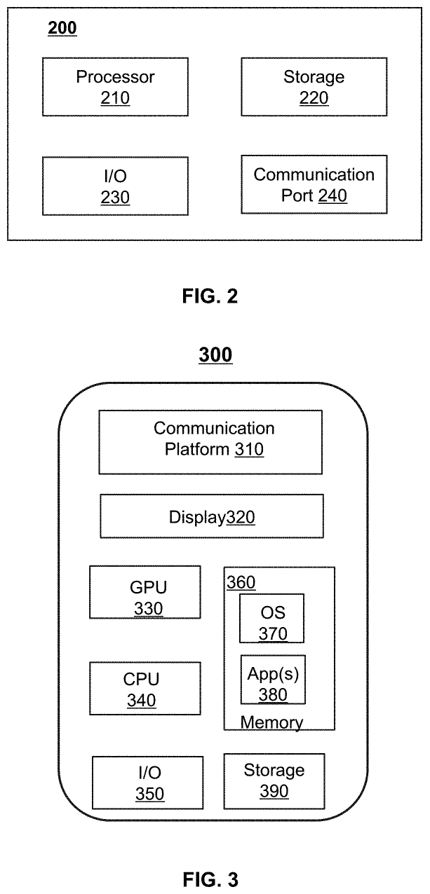

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “region,” “location,” and “area” in the present disclosure may refer to a location of an anatomical structure shown in the image or an actual location of the anatomical structure existing in or on a target subject's body, since the image may indicate the actual location of a certain anatomical structure existing in or on the target subject's body. The term “an image of a subject” may be referred to as the subject for brevity. Segmentation of an image of a subject may be referred to as segmentation of the subject.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

A conventional medical imaging procedure often involves a lot of human intervention. Merely by way of example, a user (e.g., a doctor, an operator, a technician, etc.) may need to manually perform a scan preparation for a scan of a target subject, which involves, for example, adjusting positions of a plurality of components of a medical imaging device, setting one or more scanning parameters, guiding the target subject to hold a specific posture, checking the position of the target subject, or the like. Such a medical imaging procedure may be inefficient and/or susceptible to human errors or subjectivity. Thus, it may be desirable to develop systems and methods for automated scan preparation in medical imaging, thereby improving the imaging efficiency and/or accuracy. The terms “automatic” and “automated” are used interchangeable referring to methods and systems that analyzes information and generates results with little or no direct human intervention.

The present disclosure may provide systems and methods for automated scan preparation in medical imaging. According to some embodiments of the present disclosure, a plurality of scan preparation operations may be performed automatically or semi-automatically. The plurality of scan preparation operations may include identifying a target subject to be scanned by a medical imaging device from one or more candidate subjects, generating a target posture model of the target subject, adjusting position(s) of one or more components (e.g., a scanning table, a detector, a X-ray tube, a supporting device) of the medical imaging device, setting one or more scanning parameters (e.g., a size of a light field, an estimated dose associated with the target subject), guiding the target subject to hold a specific posture, checking the position of the target subject, determining an orientation of the target subject, selecting at least one target ionization chamber, or the like, or any combination thereof. Compared with a conventional scan preparation which involves a lot of human intervention, the systems and methods of the present disclosure may be implemented with reduced or minimal or without user intervention, which is more efficient and accurate by, e.g., reducing the workload of a user, cross-user variations, and the time needed for the scan preparation.

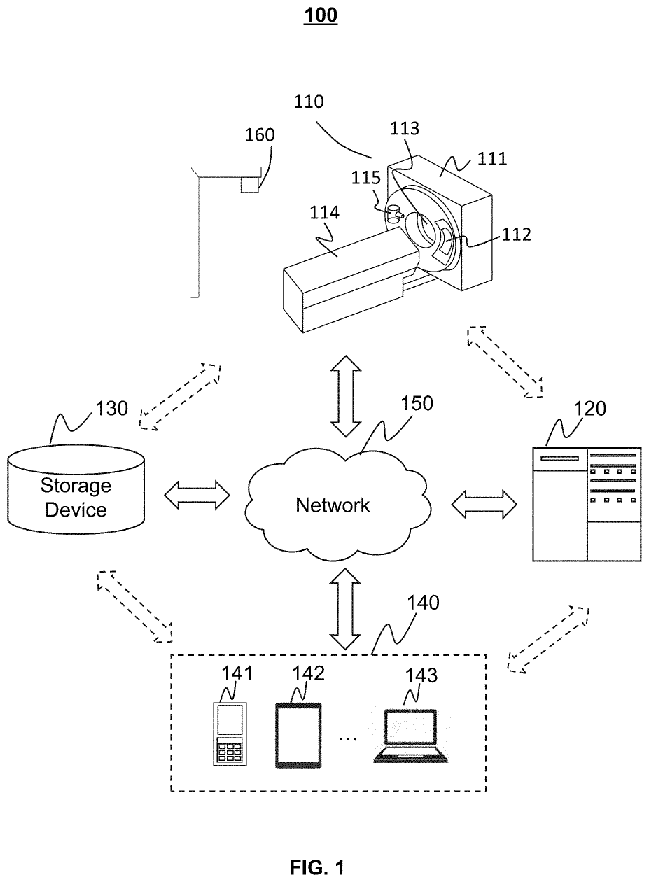

FIG. 1 is a schematic diagram illustrating an exemplary imaging system 100 according to some embodiments of the present disclosure. As shown, the imaging system 100 may include a medical imaging device 110, a processing device 120, a storage device 130, one or more terminals 140, a network 150, and an image capturing device 160. In some embodiments, the medical imaging device 110, the processing device 120, the storage device 130, the terminal(s) 140, and/or the image capturing device 160 may be connected to and/or communicate with each other via a wireless connection, a wired connection, or a combination thereof. The connection between the components of the imaging system 100 may be variable. Merely by way of example, the medical imaging device 110 may be connected to the processing device 120 through the network 150 or directly. As a further example, the storage device 130 may be connected to the processing device 120 through the network 150 or directly.

The medical imaging device 110 may generate or provide image data related to a target subject via scanning the target subject. For illustration purposes, image data of a target subject acquired using the medical imaging device 110 is referred to as medical image data, and image data of the target subject acquired using the image capturing device 160 is referred to as image data. In some embodiments, the target subject may include a biological subject and/or a non-biological subject. For example, the target subject may include a specific portion of a body, such as the head, the thorax, the abdomen, or the like, or a combination thereof. As another example, the target subject may be a man-made composition of organic and/or inorganic matters that are with or without life. In some embodiments, the imaging system 100 may include modules and/or components for performing imaging and/or related analysis. In some embodiments, the medical image data relating to the target subject may include projection data, one or more images of the target subject, etc. The projection data may include raw data generated by the medical imaging device 110 by scanning the target subject and/or data generated by a forward projection on an image of the target subject.

In some embodiments, the medical imaging device 110 may be a non-invasive biomedical medical imaging device for disease diagnostic or research purposes. The medical imaging device 110 may include a single modality scanner and/or a multi-modality scanner. The single modality scanner may include, for example, an ultrasound scanner, an X-ray scanner, an computed tomography (CT) scanner, a magnetic resonance imaging (MRI) scanner, an ultrasonography scanner, a positron emission tomography (PET) scanner, an optical coherence tomography (OCT) scanner, an ultrasound (US) scanner, an intravascular ultrasound (IVUS) scanner, a near infrared spectroscopy (NIRS) scanner, a far infrared (FIR) scanner, or the like, or any combination thereof. The multi-modality scanner may include, for example, an X-ray imaging-magnetic resonance imaging (X-ray-MRI) scanner, a positron emission tomography-X-ray imaging (PET-X-ray) scanner, a single photon emission computed tomography-magnetic resonance imaging (SPECT-MRI) scanner, a positron emission tomography-computed tomography (PET-CT) scanner, a digital subtraction angiography-magnetic resonance imaging (DSA-MRI) scanner, etc. It should be noted that the scanner described above is merely provided for illustration purposes, and not intended to limit the scope of the present disclosure. The term “imaging modality” or “modality” as used herein broadly refers to an imaging method or technology that gathers, generates, processes, and/or analyzes imaging information of a target subject.

For illustration purposes, the present disclosure mainly describes systems and methods relating to an X-ray imaging system. It should be noted that the X-ray imaging system described below is merely provided as an example, and not intended to limit the scope of the present disclosure. The systems and methods disclosed herein may be applied to any other imaging systems.

In some embodiments, the medical imaging device 110 may include a gantry 111, a detector 112, a detection region 113, a scanning table 114, and a radiation source 115. The gantry 111 may support the detector 112 and the radiation source 115. The target subject may be placed on the scanning table 114 and moved into the detection region 113 to be scanned. The radiation source 115 may emit radioactive rays to the target subject. The radioactive rays may include a particle ray, a photon ray, or the like, or a combination thereof. In some embodiments, the radioactive rays may include a plurality of radiation particles (e.g., neutrons, protons, electron, p-mesons, heavy ions), a plurality of radiation photons (e.g., X-ray, y-ray, ultraviolet, laser), or the like, or a combination thereof. The detector 112 may detect radiation and/or a radiation event (e.g., gamma photons) emitted from the detection region 113. In some embodiments, the detector 112 may include a plurality of detector units. The detector units may include a scintillation detector (e.g., a cesium iodide detector) or a gas detector. The detector unit may be a single-row detector or a multi-rows detector.

In some embodiments, the medical imaging device 110 may be or include an X-ray imaging device, for example, a computed tomography (CT) scanner, a digital radiography (DR) scanner (e.g., a mobile digital radiography), a digital subtraction angiography (DSA) scanner, a dynamic spatial reconstruction (DSR) scanner, an X-ray microscopy scanner, a multimodality scanner, etc. For example, the X-ray imaging device may include a support, an X-ray source, and a detector. The support may be configured to support the X-ray source and/or the detector. The X-ray source may be configured to emit X-rays toward the target subject to be scanned. The detector may be configured to detect X-rays passing through the target subject. In some embodiments, the X-ray imaging device may be, for example, a C-shape X-ray imaging device, an upright X-ray imaging device, a suspended X-ray imaging device, or the like.

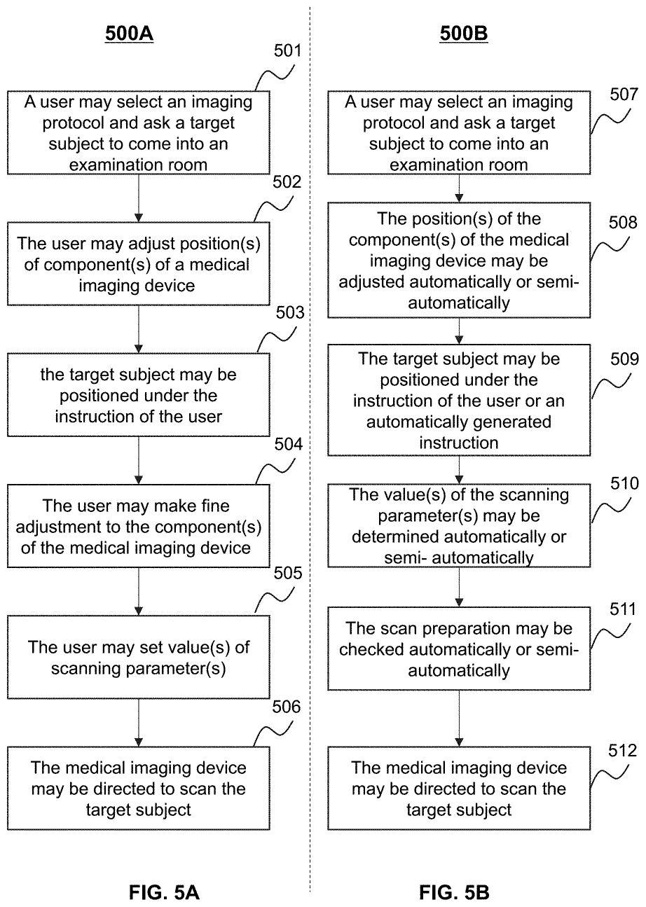

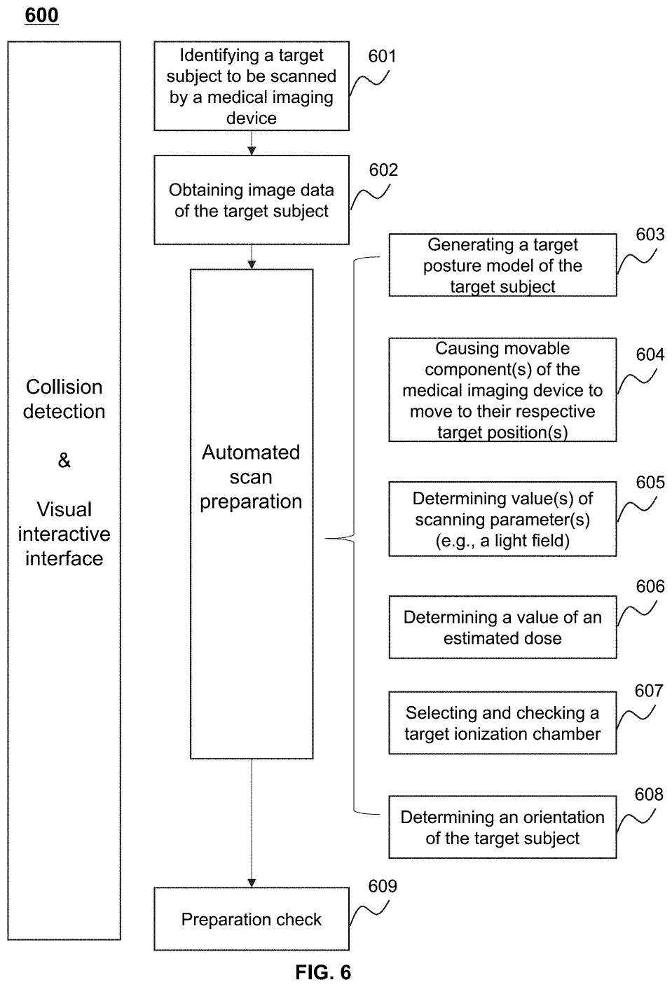

The processing device 120 may process data and/or information obtained from the medical imaging device 110, the storage device 130, the terminal(s) 140, and/or the image capturing device 160. For example, the processing device 120 may implement an automated scan preparation for a scan to be performed on a target subject. The automated scan preparation may include, for example, identifying the target subject to be scanned, generating a target posture model of the target subject, causing a movable component of the medical imaging device 110 to move to its target position, determining one or more scanning parameters (e.g., a size of a light field), or the like, or any combination thereof. More descriptions regarding the automated scan preparation may be found elsewhere in the present disclosure. See, e.g., FIGS. 5 and 6 and relevant descriptions thereof.

In some embodiments, the processing device 120 may be a single server or a server group. The server group may be centralized or distributed. In some embodiments, the processing device 120 may be local to or remote from the imaging system 100. For example, the processing device 120 may access information and/or data from the medical imaging device 110, the storage device 130, the terminal(s) 140, and/or the image capturing device 160 via the network 150. As another example, the processing device 120 may be directly connected to the medical imaging device 110, the terminal(s) 140, the storage device 130, and/or the image capturing device 160 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be implemented by a computing device 200 having one or more components as described in connection with FIG. 2.

In some embodiments, the processing device 120 may include one or more processors (e.g., single-core processor(s) or multi-core processor(s)). Merely by way of example, the processing device 120 may include a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

The storage device 130 may store data, instructions, and/or any other information. In some embodiments, the storage device 130 may store data obtained from the processing device 120, the terminal(s) 140, the medical imaging device 110, and/or the image capturing device 160. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform as described elsewhere in the disclosure.

In some embodiments, the storage device 130 may be connected to the network 150 to communicate with one or more other components of the imaging system 100 (e.g., the processing device 120, the terminal(s) 140). One or more components of the imaging system 100 may access the data or instructions stored in the storage device 130 via the network 150. In some embodiments, the storage device 130 may be part of the processing device 120.

The terminal(s) 140 may enable user interaction between a user and the imaging system 100. For example, the terminal(s) 140 may display a composite image in which the target subject and a target posture model of the target subject are overplayed. In some embodiments, the terminal(s) 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal(s) 140 may include an input device, an output device, etc. In some embodiments, the terminal(s) 140 may be part of the processing device 120.

The network 150 may include any suitable network that can facilitate the exchange of information and/or data for the imaging system 100. In some embodiments, one or more components of the imaging system 100 (e.g., the medical imaging device 110, the processing device 120, the storage device 130, the terminal(s) 140) may communicate information and/or data with one or more other components of the imaging system 100 via the network 150. For example, the processing device 120 may obtain medical image data from the medical imaging device 110 via the network 150. As another example, the processing device 120 may obtain user instruction(s) from the terminal(s) 140 via the network 150.

The network 150 may be or include a public network (e.g., the Internet), a private network (e.g., a local area network (LAN)), a wired network, a wireless network (e.g., an 802.11 network, a Wi-Fi network), a frame relay network, a virtual private network (VPN), a satellite network, a telephone network, routers, hubs, switches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the imaging system 100 may be connected to the network 150 to exchange data and/or information.

The image capturing device 160 may be configured to capture image data of the target subject before, during, and/or after the medical imaging device 110 performs a scan on the target subject. For example, before the scan, the image capturing device 160 may capture first image data of the target subject, which may be used to generate a target posture model of the target subject and/or determine one or more scanning parameters of the medical imaging device 110. As another example, after the target subject is positioned at a scan position (i.e., a specific position for receiving the scan), the image capturing device 160 may be configured to capture second image data of the target subject, which may be used to check whether the posture and/or position of the target subject needs to be adjusted.

The image capturing device 160 may be and/or include any suitable device that is capable of capturing image data of the target subject. For example, the image capturing device 160 may include a camera (e.g., a digital camera, an analog camera, etc.), a red-green-blue (RGB) sensor, an RGB-depth (RGB-D) sensor, or another device that can capture color image data of the target subject. As another example, the image capturing device 160 may be used to acquire point-cloud data of the target subject. The point-cloud data may include a plurality of data points, each of which may represent a physical point on a body surface of the target subject and can be described using one or more feature values of the physical point (e.g., feature values relating to the position and/or the composition of the physical point). Exemplary image capturing devices 160 capable of acquiring point-cloud data may include a 3D scanner, such as a 3D laser imaging device, a structured light scanner (e.g., a structured light laser scanner). Merely by way of example, a structured light scanner may be used to execute a scan on the target subject to acquire the point cloud data. During the scan, the structured light scanner may project structured light (e.g., a structured light spot, a structured light grid) that has a certain pattern toward the target subject. The point-cloud data may be acquired according to the structure light projected on the target subject. As yet another example, the image capturing device 160 may be used to acquire depth image data of the target subject. The depth image data may refer to image data that includes depth information of each physical point on the body surface of the target subject, such as a distance from each physical point to a specific point (e.g., an optical center of the image capturing device 160). The depth image data may be captured by a range sensing device, e.g., a structured light scanner, a time-of-flight (TOF) device, a stereo triangulation camera, a sheet of light triangulation device, an interferometry device, a coded aperture device, a stereo matching device, or the like, or any combination thereof.

In some embodiments, the image capturing device 160 may be a device independent from the medical imaging device 110 as shown in FIG. 1. For example, the image capturing device 160 may be a camera mounted on the ceiling in an examination room where the medical imaging device 110 is located or out of the examination room. Alternatively, the image capturing device 160 may be integrated into or mounted on the medical imaging device 110 (e.g., the gantry 111). In some embodiments, the image data acquired by the image capturing device 160 may be transmitted to the processing device 120 for further analysis. Additionally or alternatively, the image data acquired by the image capturing device 160 may be transmitted to a terminal device (e.g., the terminal(s) 140) for display and/or a storage device (e.g., the storage device 130) for storage.

In some embodiments, the image capturing device 160 may be configured to capture image data of the target subject continuously or intermittently (e.g., periodically) before, during, and/or after a scan of the target subject performed by the medical imaging device 110. In some embodiments, the acquisition of the image data by the image capturing device 160, the transmission of the captured image data to the processing device 120, and the analysis of the image data may be performed substantially in real time so that the image data may provide information indicating a substantially real time status of the target subject.

It should be noted that the above description of the imaging system 100 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the imaging system 100 may include one or more additional components. Additionally or alternatively, one or more components of the imaging system 100, such as the image capturing device 160 or the medical imaging device 110 described above may be omitted. As another example, two or more components of the imaging system 100 may be integrated into a single component. Merely by way of example, the processing device 120 (or a portion thereof) may be integrated into the medical imaging device 110 or the image capturing device 160.

FIG. 2 is a schematic diagram illustrating exemplary hardware and/or software components of a computing device 200 according to some embodiments of the present disclosure. The computing device 200 may be used to implement any component of the imaging system 100 as described herein. For example, the processing device 120 and/or the terminal 140 may be implemented on the computing device 200, respectively, via its hardware, software program, firmware, or a combination thereof. Although only one such computing device is shown, for convenience, the computer functions relating to the imaging system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 120 in accordance with techniques described herein. The computer instructions may include, for example, routines, programs, subjects, components, data structures, procedures, modules, and functions, which perform particular functions described herein. For example, the processor 210 may process image data obtained from the medical imaging device 110, the terminal(s) 140, the storage device 130, the image capturing device 160, and/or any other component of the imaging system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information obtained from the medical imaging device 110, the terminal(s) 140, the storage device 130, the image capturing device 160, and/or any other component of the imaging system 100. In some embodiments, the storage device 220 may include a mass storage device, a removable storage device, a volatile read-and-write memory, a read-only memory (ROM), or the like, or any combination thereof. In some embodiments, the storage device 220 may store one or more programs and/or instructions to perform exemplary methods described in the present disclosure. For example, the storage device 220 may store a program for the processing device 120 to execute to perform an automated scan preparation for a scan to be performed on a target subject.

The I/O 230 may input and/or output signals, data, information, etc. In some embodiments, the I/O 230 may enable a user interaction with the processing device 120. In some embodiments, the I/O 230 may include an input device and an output device. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to another component (e.g., the processing device 120) via, for example, a bus, for further processing. Other types of the input device may include a cursor control device, such as a mouse, a trackball, or cursor direction keys, etc. The output device may include a display (e.g., a liquid crystal display (LCD), a light-emitting diode (LED)-based display, a flat panel display, a curved screen, a television device, a cathode ray tube (CRT), a touch screen), a speaker, a printer, or the like, or a combination thereof.

The communication port 240 may be connected to a network (e.g., the network 150) to facilitate data communications. The communication port 240 may establish connections between the processing device 120 and the medical imaging device 110, the terminal(s) 140, the image capturing device 160, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee™ link, a mobile network link (e.g., 3G, 4G, 5G), or the like, or a combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of a mobile device 300 according to some embodiments of the present disclosure. In some embodiments, one or more components (e.g., a terminal 140 and/or the processing device 120) of the imaging system 100 may be implemented on the mobile device 300.

As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to the imaging system 100. User interactions with the information stream may be achieved via the I/O 350 and provided to the processing device 120 and/or other components of the imaging system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

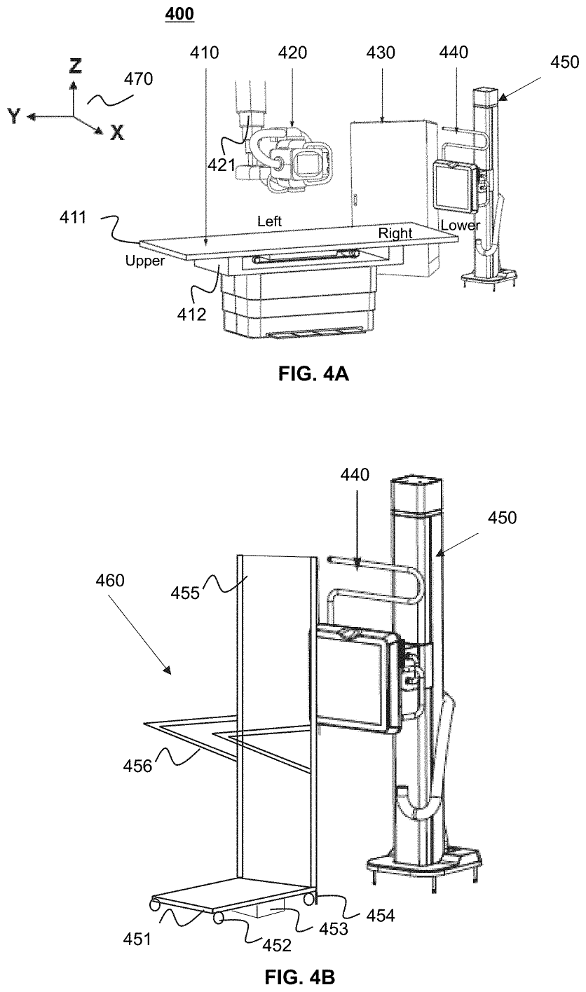

FIG. 4A is a schematic diagram illustrating an exemplary medical imaging device 400 according to some embodiments of the present disclosure. FIG. 4B is a schematic diagram illustrating an exemplary supporting device 460 of the medical imaging device 400 according to some embodiments of the present disclosure. The medical imaging device 400 may be an exemplary embodiment of the medical imaging device 110 as described in connection with FIG. 1. As shown in FIG. 4A, the medical imaging device 400 may be a suspended digital radiography device. The medical imaging device 400 may include a scanning table 410, an X-ray source 420, a suspension device 421, a control apparatus 430, a flat panel detector 440, and a column 450.

In some embodiments, the scanning table 410 may include a supporting component 411 and a driving component 412. The supporting component 411 may be configured to support a target subject to be scanned. The driving component 412 may be configured to drive the supporting component to move by, e.g., translating and/or rotating. The positive direction of the X axis of the coordinate system 470 indicates the direction from the left edge to the right edge of the scanning table 410 (or the supporting component 411). The positive direction of the Y axis of the coordinate system 470 indicates the direction from the lower edge to the upper edge of the scanning table 410 (or the supporting component 411).

The suspension device 421 may be configured to suspend the X-ray source 420 and control the X-ray source 420 to move. For example, the suspension device 421 may control the X-ray source 420 to move to adjust the distance between the X-ray source 420 and the flat panel detector 440. In some embodiments, the X-ray source 420 may include an X-ray tube and a beam limiting device (not shown in FIG. 4A). The X-ray tube may be configured to emit one or more X-rays toward the target subject to be scanned. The beam limiting device may be configured to control an irradiation region of the X-rays on the target subject. Additionally or alternatively, the beam limiting device may be configured to adjust the intensity and/or the amount of the X-rays that irradiate on the target subject. In some embodiments, a handle may be mounted on the X-ray source 420. A user may grasp the handle to move the X-ray source 420 to a desirable position.

The flat panel detector 440 may be detachably mounted on and supported by the column 450. In some embodiments, the flat panel detector 440 may move with respect to the column 450 by, for example, translating along the column 450 and/or rotating around the column 450. The control apparatus 430 may be configured to control one or more components of the medical imaging device 400. For example, the control apparatus 430 may control the X-ray source 420 and the flat panel detector 440 to move to their respective target positions.

In some embodiments, the scanning table 410 of the medical imaging device 400 may be replaced by a supporting device 460 as shown in FIG. 4B. The supporting device 460 may be used to support a target subject who holds an upright posture when the medical imaging device 400 scans the target subject. For example, the target subject may stand, sit, or kneel on the supporting device 460 to receive a scan. In some embodiments, the supporting device 460 may be used in a stitching scan of the target subject. A stitching scan refers to a scan in which a plurality of regions of the target subject may be scanned in sequence to acquire a stitched image of the regions. For instance, an image of the whole body of the target subject may be obtained by performing a plurality of scans of various portions of the target subject in sequence in a stitching scan.

In some embodiments, the supporting device 460 may include a supporting component 451, a first driving component 452, a second driving component 453, a fixing component 454, and a panel 455. The supporting component 451 may be configured to support the target subject. In some embodiments, the supporting component 451 may be a flat plate made of any suitable material that has high strength and/or stability to provide a stable support for the target subject. The first driving component 452 may be configured to drive the supporting device to move in a first direction (e.g., on an X-Y plane of a coordinate system 470 as shown in FIG. 4A). In some embodiments, the first driving component 451 may be a roller, a wheel (e.g., a universal wheel), or the like. For example, the supporting device 460 may move around on the ground via the wheels.

The second driving component 453 may be configured to drive the supporting component 451 to move along a second direction. The second direction may be perpendicular to the first direction. For example, the first direction may be parallel to the X-Y plane of the coordinate system 470, and the second direction may be parallel to a Z-axis direction of the coordinate system 470. In some embodiments, the second driving component 453 may be a lifting device. For example, the second driving component 453 may be a scissors arm, a rod type lifting device (e.g., a hydraulic rod lifting device), or the like. The fixing component 454 may be configured to fix the supporting device 460 at a certain position. For example, the fixing component 454 may be a column, a bolt, or the like.

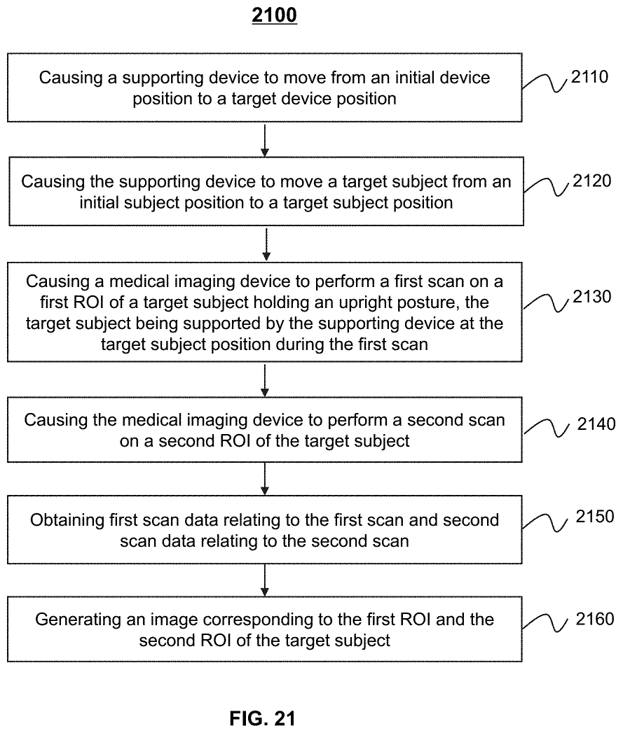

The panel 455 may be located between the target subject and one or more other components of the medical imaging device 400 during the scan of the target subject. The panel 455 may be configured to separate the target subject from the one or more components (e.g., the flat panel detector 440) of the medical imaging device 400 to avoid a collision between the target subject and the one or more components (e.g., the flat panel detector 440) of the medical imaging device 400. In some embodiments, the panel 455 may be made of any material that is transparent to light and has a relatively low X-ray absorption rate (e.g., the X-ray absorption rate lower than a threshold). In such cases, the panel 455 may exert little or no interference on the reception of X-ray by the flat panel detector 440, e.g., X-ray beams emitted by an X-ray tube that have traversed the target subject or not. For example, the panel 455 may be made of polymethyl methacrylate (PMMA), polyethylene (PE), polyvinyl chloride (PVC), polystyrene (PS), high impact polystyrene (HIPS), polypropylene (PP), acrylonitrile butadiene-styrene (ABS) resin, or the like, or any combination thereof. In some embodiments, the panel 455 may be fixed on the supporting component 451 using an adhesive, a threaded connection, a lock, a bolt, or the like, or any combination thereof. More descriptions regarding the supporting device 460 may be found elsewhere in the present disclosure (e.g., FIG. 21 and the relevant descriptions thereof).

In some embodiments, the supporting device 460 may further include one or more handles 456. The target subject may grasp the one or more handles 456 when he/she gets on and/or gets off the supporting device 460. The target subject may also grab the one or more handles 456 when the supporting device 460 moves the target subject from one scan position to another scan position. In some embodiments, the one or more handles 456 may be movable. For example, the handle(s) 456 may move along the Z-axis direction of the coordinate system 470 as shown in FIG. 4A. In some embodiments, the position of the handle(s) 456 may be adjusted automatically according to, for example, the height of the target subject, such that the target subject may easily grab the handle(s). More descriptions regarding a supporting device may be found elsewhere in the present disclosure. See, e.g., FIG. 21 and relevant descriptions thereof.

It should be noted that the examples illustrated in FIGS. 4A and 4B are provided for the purposes of illustration, and not intended to limit the scope of the present disclosure. For persons having ordinary skills in the art, various modifications and changes in the forms and details of the application of the above method and system may occur without departing from the principles of the present disclosure. In some embodiments, the column 450 may be configured in any suitable manner, such as a C-shaped support, a U-shape support, a G-shape support, or the like. In some embodiments, the medical imaging device 400 may include one or more additional components not described and/or without one or more components illustrated in FIGS. 4A and 4B. For example, the medical imaging device 400 may further include a camera. As another example, two or more components of the medical imaging device 400 may be integrated into a single component. Merely by way of example, the first driving component 452 and the second driving component 453 may be integrated into a single driving component.

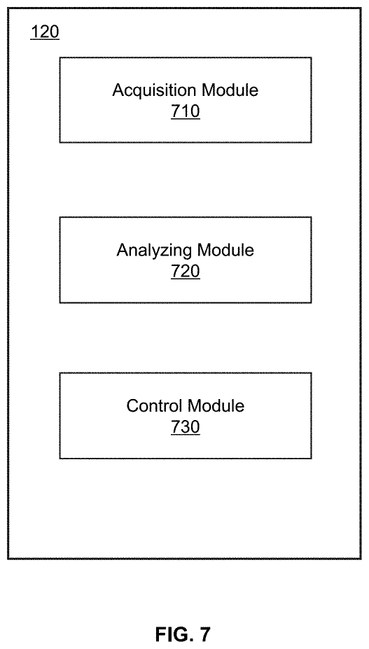

FIG. 5A is a flowchart illustrating a traditional process 500A for scanning a target subject. FIG. 5B is a flowchart illustrating an exemplary process 500B for scanning a target subject according to some embodiments of the present disclosure. In some embodiments, the process 500B may be implemented in the imaging system 100 illustrated in FIG. 1. For example, the process 500B may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 800 may be accomplished with one or more additional operations not described, and/or without one or more of the operations discussed. Additionally, the order in which the operations of the process 500B as illustrated in FIG. 5B and described below is not intended to be limiting.

As illustrated in FIG. 5A, the traditional scanning process of the target subject may include operations 501 to 506.

In 501, a user may select an imaging protocol and ask the target subject to come into an examination room.

For example, the target subject may be a patient to be imaged (or treated) by a medical imaging device (e.g., the medical imaging device 110) in the examination room. In some embodiments, the user (e.g., a doctor, an operator, a technician, etc.) may call an examination number and/or a name of the target subject to ask the target subject come into the examination room. In some embodiments, the user may select the imaging protocol based on equipment parameters of the medical imaging device, the user's preference, and/or information associated with the target subject (e.g., a body shape of the target subject, the gender of the target subject, a portion of the target subject to be imaged, etc.).

In 502, the user may adjust position(s) of component(s) of the medical imaging device.

The medical imaging device (e.g., the medical imaging device 110) may be an X-ray imaging device (e.g., a suspended X-ray imaging device, a C-arm X-ray imaging device), a digital radiography (DR) device (e.g., a mobile digital X-ray imaging device), a CT device, a PET device, an MRI device, or the like, as described elsewhere in the present disclosure. Merely by way of example, for an X-ray imaging device, the one or more components of the X-ray imaging device may include a scanning table (e.g., the scanning table 114), a detector (e.g., the detector 112, the flat panel detector 440), an X-ray source (e.g., the radiation source 115, the X-ray source 420), a supporting device (e.g., the supporting device 460), or the like. In some embodiments, the user may input position parameter(s) of a component according to the imaging protocol via a terminal device. Additionally or alternatively, the user may manually move a component of the medical imaging device to a suitable position.

In 503, the target subject may be positioned under the instruction of the user.

In some embodiments, the target subject may need to hold a standard posture (also referred to as a reference posture) during the scan to be performed on the target subject. The user may instruct the target subject to stand or lie on a specific position and hold a specific pose. In some embodiments, after the target subject is positioned at a scan position (i.e., a specific position for receiving the scan), the user may check the posture and/or the position of the target subject, and/or instruct the target subject to adjust his/her posture and/or position if needed.

In 504, the user may make fine adjustment to the component(s) of the medical imaging device.

In some embodiments, after the target subject is positioned at the scan position, the user may further check and/or adjust the position(s) of one or more components of the medical imaging device. For example, the user may determine whether the position of the detector needs to be adjusted based on the scan position and the posture of the target subject.

In 505, the user may set value(s) of scanning parameter(s).

The scanning parameter(s) may include an X-ray tube voltage and/or current, a scan mode, a table moving speed, a gantry rotation speed, a field of view (FOV), a scan time, a size of a light field, or the like, or any combination thereof. In some embodiments, the user may set the value(s) of the scanning parameter(s) based on the imaging protocol, the information associated with the target subject, or the like, or any combination thereof.

In 506, the medical imaging device may be directed to scan the target subject.

In some embodiments, medical image data of the target subject may be acquired during the scan of the target subject by the medical imaging device. The user may perform one or more image processing operations on the medical image data. For example, the user may perform an image segmentation operation, an image classification operation, an image scaling operation, an image rotation operation, or the like, on the medical image data.

As illustrated in FIG. 5B, an exemplary process 500B for scanning the target subject according to some embodiments of the present disclosure may include one or more of operations 507 to 512.

In 507, a user may select an imaging protocol and ask a target subject to come into an examination room.

Operation 507 may be performed in a similar manner with operation 501 as described in connection with FIG. 5A, and the descriptions thereof are not repeated here. In some embodiments, the processing device 120 may select the imaging protocol according to, for example, the portion of the target subject to be scanned and/or other information of the target subject. Additionally or alternatively, the processing device 120 may cause a terminal device to output a notification to ask the target subject to come into the examination room.

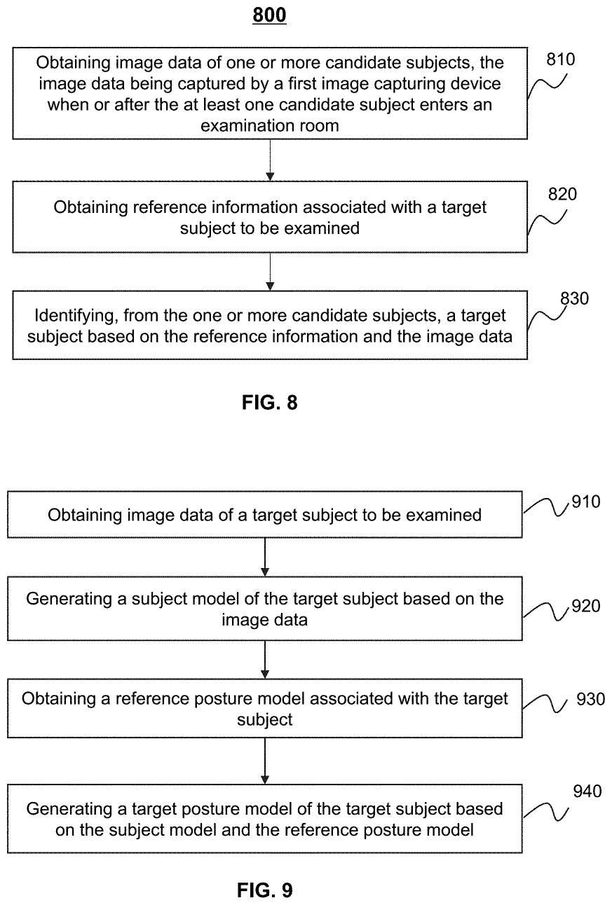

In some embodiments, one or more candidate subjects may enter the examination room. The processing device 120 may identify the target subject from the one or more candidate subjects automatically or semi-automatically. For example, the processing device 120 may obtain image data of the one or more candidate subjects when or after the one or more candidate subjects enter the examination room. The image data may be captured by an image capturing device mounted in or out of the examination room. The processing device 120 may automatically identify, from the one or more candidate subjects, the target subject based on reference information associated with the target subject and the image data of the one or more candidate subjects. More descriptions of the identification of the target subject may be found elsewhere in the present disclosure (e.g., FIG. 8 and descriptions thereof).

In 508, the position(s) of the component(s) of the medical imaging device may be adjusted automatically or semi-automatically.

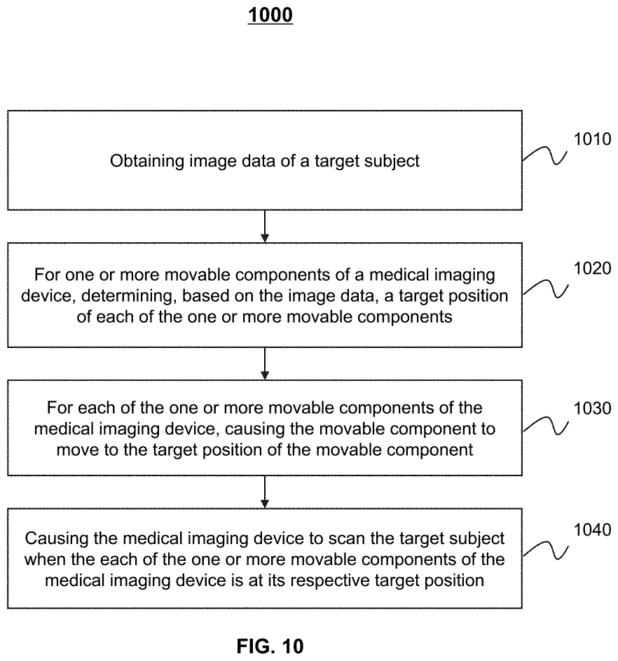

In some embodiments, the processing device 120 may determine the position(s) of the component(s) of the medical imaging device based on image data of the target subject. For example, the processing device 120 may obtain the image data of the target subject from an image capturing device mounted in the examination room. The processing device 120 may then generate a subject model (or a target posture model) representing the target subject based on the image data of the target subject. The processing device 120 may further determine a target position of a component (e.g., a detector, a scanning table, a supporting device) of the medical imaging device based on the subject model (or the target posture model). More descriptions for determining a target position for a component of the medical imaging device may be found elsewhere in the present disclosure (e.g., FIGS. 10, 11, and 21 and descriptions thereof).

In 509, the target subject may be positioned under the instruction of the user or an automatically generated instruction.

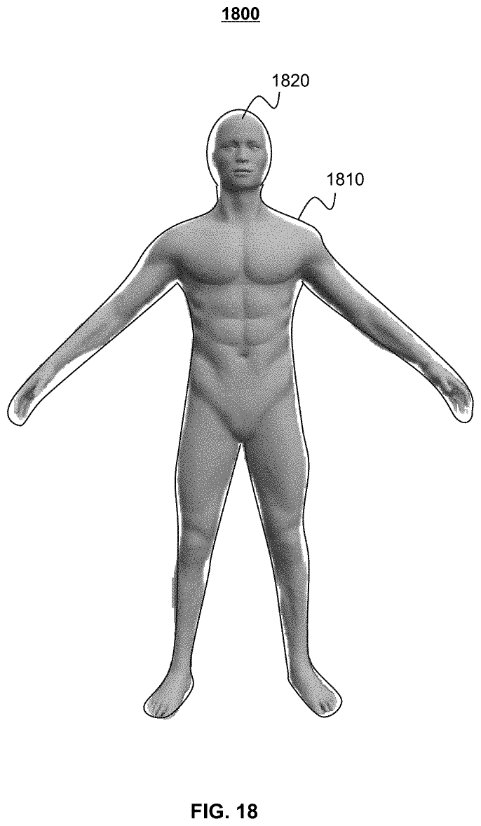

In some embodiments, after the target subject is positioned at the scan position, the processing device 120 may obtain target image data of the target subject holding a posture. The processing device 120 may determine whether the posture of the target subject needs to be adjusted based on the target image data and a target posture model. If it is determined that the posture of the target subject needs to be adjusted, the processing device 120 may further cause an instruction to be generated. The instruction may guide the target subject to move one or more body parts of the target subject to hold the target posture. More descriptions for the positioning of the target subject may be found elsewhere in the present disclosure (e.g., FIG. 17 and descriptions thereof).

In some embodiments, if the target subject holds an upright posture to receive the scan, the position of a detector of the medical imaging device (e.g., the flat panel detector 440 as shown in FIG. 4B) may be adjusted first. Then the target subject may be asked to stand at a specific scan position to receive the scan (for example, stands on the supporting component 451 as shown in FIG. 4B to receive the scan), and a radiation source of the medical imaging device may be adjusted after the target subject stands at the specific scan position. If the target subject lies on a scanning table of the medical imaging device to receive the scan, the target subject may be asked to lie on the scanning table first, and then the radiation source and the detector may be adjusted to their respective target positions. This may avoid a collision between the target subject, the detector, and the radiation source.

In 510, value(s) of the scanning parameter(s) may be determined automatically or semi-automatically.

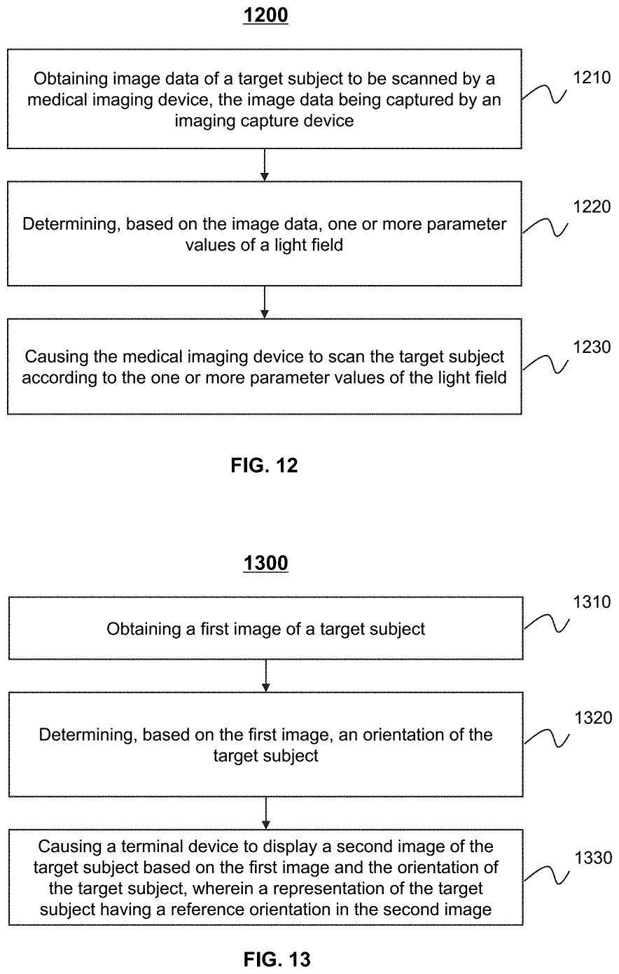

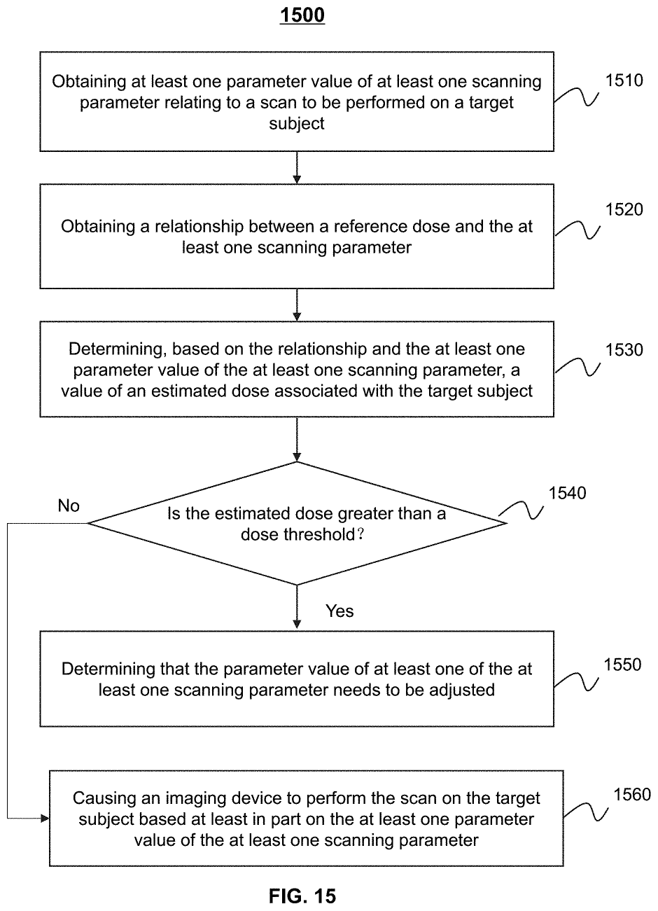

In some embodiments, the processing device 120 may determine the value(s) of the scanning parameter(s) based on feature information (e.g., the width, the thickness, the height) relating to a region of interest (ROI) of the target subject. An ROI of the target subject refers to a scanning region or a portion thereof (e. a specific organ or tissue in the scanning region) of the target subject to be imaged (or examined or treated). For example, the processing device 120 may determine the feature information relating to the ROI of the target subject based on the image data of the target subject or the subject model (or the target posture model) of the target subject generated based on the image data. The processing device 120 may further determine values of a voltage of a radiation source, a current of a radiation source and/or an exposure time of the scan based on the thickness of the ROI. Additionally or alternatively, the processing device 120 may determine a target size of a light field based on the width and the height of the ROI of the target subject. More descriptions of the determination of the value(s) of the scanning parameter(s) may be found elsewhere in the present disclosure (e.g., FIGS. 12 and 15 and descriptions thereof).

In 511, the scan preparation may be checked automatically or semi-automatically.

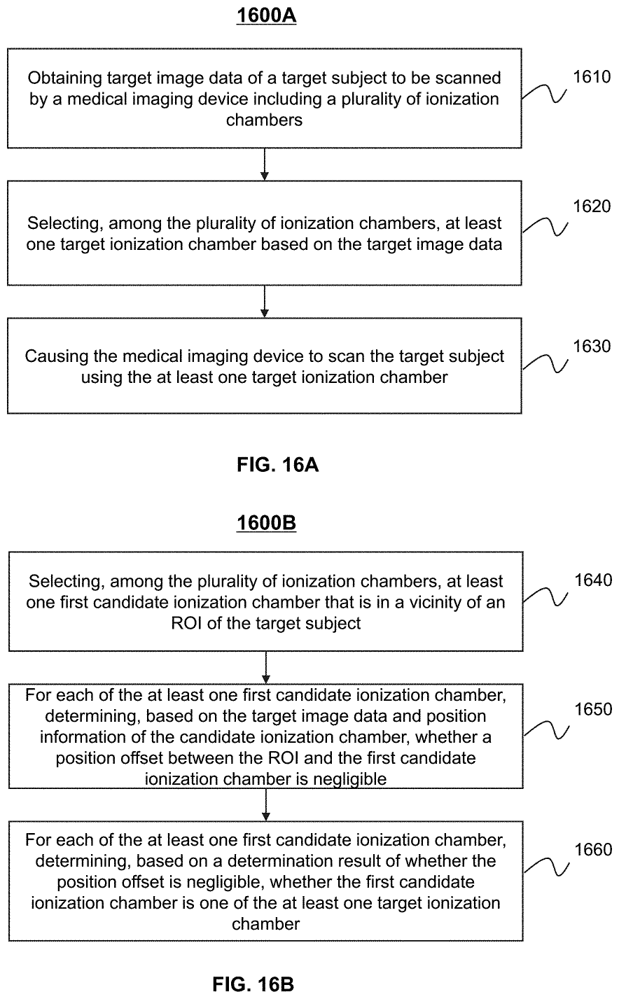

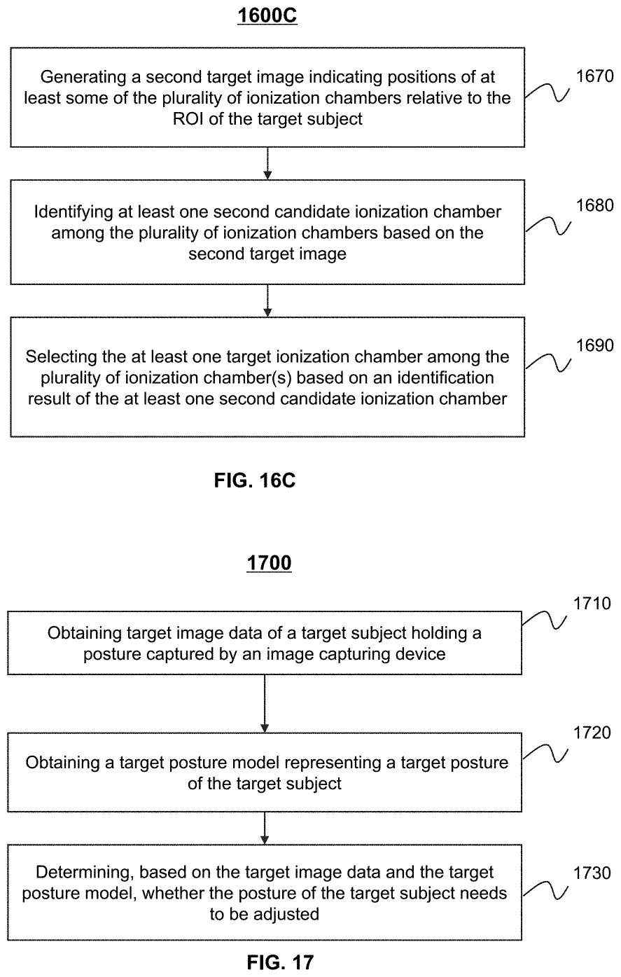

In some embodiments, the position(s) of the component(s) determined in operation 508, the position and/or the posture of the target subject, and/or the value(s) of the scanning parameter(s) determined in operation 510 may be further checked and/or adjusted. For example, the position of a movable component may be manually checked and/or adjusted by a user of the imaging system 100. As yet another example, after the target subject is positioned at the scan position, target image data of the target subject may be captured using an image capturing device. The target position of a movable component (e.g., the detector) may be automatically checked and/or adjusted by one or more components (e.g., the processing device 120) of the imaging system 100 based on the target image data. More descriptions regarding the scan preparation check based on the target image data may be found elsewhere in the present disclosure. See, e.g., FIGS. 16, 17, and 19 and relevant descriptions thereof.

In 512, the medical imaging device may be directed to scan the target subject

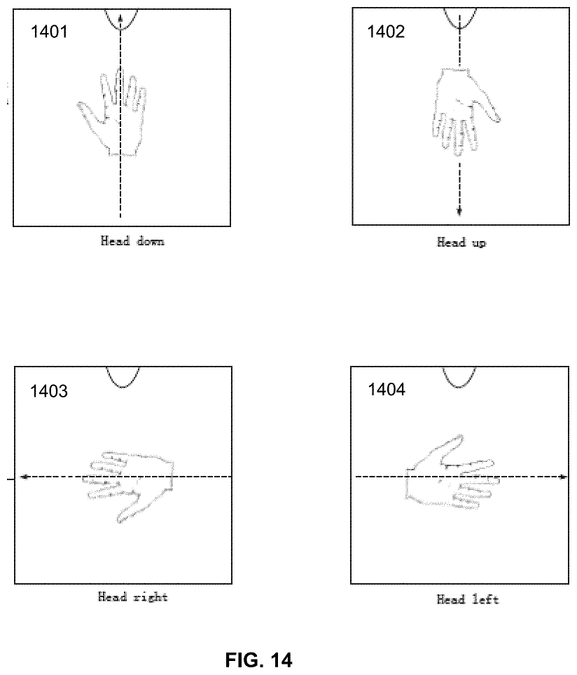

In some embodiments, medical image data of the target subject may be acquired during the scan of the target subject by the medical imaging device. The processing device 120 may perform one or more additionally operations to process the medical image data. For example, the processing device 120 may determine an orientation of the target subject based on the medical image data, and display the medical image data according to the orientation of the target subject. More descriptions for the determination of the orientation of the target subject may be found elsewhere in the present disclosure (e.g., FIGS. 13 and 14 and descriptions thereof).

It should be noted that the above description of the process 500B is merely provided for the purposes of illustration, and not intended to limit the scope of the present disclosure. In some embodiments, one or more additional operations may be added, and/or one or more operations described above may be omitted. Merely by way of example, operation 511 may be omitted. Additionally or alternatively, the order of the operations of the process 500B may be modified according to an actual need. For example, operations 508-510 may be performed in any order.

FIG. 6 is a flowchart illustrating an exemplary process for scan preparation according to some embodiments of the present disclosure. Process 600 may be an exemplary embodiment of the process 500B as described in connection with FIG. 5.

In 601, the processing device 120 (e.g., the analyzing module 720) may identify a target subject to be scanned by a medical imaging device. More descriptions of the identification of the target subject may be found elsewhere in the present disclosure (e.g., FIG. 8 and descriptions thereof).

In 602, the processing device 120 (e.g., the acquisition module 710) may obtain image data of the target subject.

The image data may include a 2D image, a 3D image, a 4D image (e.g., a time series of 3D images), and/or any related image data (e.g., scan data, projection data) of the target subject. The image data may include color image data, point-cloud data, depth image data, mesh data, medical image data, or the like, or any combination thereof, of the target subject.

In some embodiments, the image data obtained in 602 may include one or more sets of image data, for example, a plurality of images of the target subject captured at a plurality of time points by an image capturing device (e.g., the image capturing device 160), a plurality of images of the target subject captured by different image capturing devices. For example, the image data may include a first set of image data capture by a specific image capturing device before the target subject is positioned at a scan position. Additionally or alternatively, the image data may include a second set of image data (also referred to as target image data) capture by the specific image capturing device (or another image capturing device) after the target subject is positioned at a scan position.

The processing device 120 may then perform an automated scan preparation. The automated scan preparation may include one or more one or more preparation operations, such as, one or more of operations 603 to 608 as shown in FIG. 6. In some embodiments, the automated scan preparation may include a plurality of preparation operations. Different preparation operations may be performed based on a same set of image data or different sets of image data of the target subject captured by one or more image capturing devices. For example, a target posture model of the target subject as described in operation 603, target position(s) of movable component(s) of the medical imaging device as described in operation 604, value(s) of scanning parameter(s) as described in operation 605 may be determined based on a same set of image data or different sets of image data of the target subject captured before the target subject is positioned at the scan position. As another example, target ionization chamber(s) as described in operation 607 may be selected based on a set of image data of the target subject captured after the target subject is positioned at the scan position.

For the convenience of descriptions, the terms “image data of a target subject” used in detail descriptions regarding different preparation operations (e.g., different processes in FIGS. 8 to 21) refer to a same set of image data or different sets of image data of the target subject unless the context clearly indicates otherwise.

In 603, the processing device 120 (e.g., the analyzing module 720) may generate a target posture model of the target subject.

As used herein, a target posture model of the target subject refers to a model representing the target subject holding a target posture (or referred to as a reference posture). The target posture may a standard posture that the target subject needs to hold during the scan to be performed on the target subject. More descriptions of the generation of the target posture model of the target subject may be found elsewhere in the present disclosure (e.g., FIG. 9, and descriptions thereof).

In 604, the processing device 120 (e.g., the control module 730) may cause movable component(s) of the medical imaging device to move to their respective target position(s).

For example, the processing device 120 may determine a target position of a movable component (e.g., a scanning table) by determining a size (e.g., a height, a width, a thickness) of the target subject based on the image data obtained in 602, especially when the target subject is positioned almost completely. Additionally or alternatively, the processing device 120 may determine a target position of a movable component (e.g., a detector, a supporting device) by generating the subject model (or the target posture model) based on the image data of the target subject. More descriptions of the determination the target position of a movable component of the medical imaging device may be found elsewhere in the present disclosure (e.g., FIGS. 10, 11A, 11B, 21, and descriptions thereof).

In 605, the processing device 120 (e.g., the analyzing module 720) may determine value(s) of scanning parameter(s) (e.g., a light field).

Operation 605 may be performed in a similar manner with operation 510, and the descriptions thereof are not repeated here.

In 606, the processing device 120 (e.g., the analyzing module 720) may determine a value of an estimated dose.

In some embodiments, the processing device 120 may obtain a relationship between a reference dose and one or more specific scanning parameters (e.g., a voltage of a radiation source, a current of a radiation source, an exposure time, etc.). The processing device 120 may determine a value of an estimated dose associated with the target subject based on the obtained relationship and parameter value(s) of the specific scanning parameter(s). More descriptions of the determination of the value of the estimated does may be found elsewhere in the present disclosure (e.g., FIG. 15 and descriptions thereof).

In 607, the processing device 120 (e.g., the analyzing module 720) may select at least one target ionization chamber.

In some embodiments, the processing device 120 may include a plurality of ionization chambers. When necessary, the at least one target ionization chamber may be actuated during the scan of the target subject, while other ionization chamber(s) (if any) may be shut down during the scan. More descriptions of the selection of the at least one target ionization chamber may be found elsewhere in the present disclosure (e.g., FIG. 16 and descriptions thereof).

In 608, the processing device 120 (e.g., the analyzing module 720) may determine an orientation of the target subject.

In some embodiments, the processing device 120 may determine an orientation of a target region corresponding to the ROI of the target subject in the image data obtained in 601. The processing device 120 may further determine the orientation of the target subject based on the orientation of the target region. In some embodiments, the processing device 120 may determine a position of the target region corresponding to the ROI of the target subject in the image data, and determine the orientation of the target subject based on the position of the target region. More descriptions for determining the orientation of the target subject may be found elsewhere in the present disclosure (e.g., FIG. 12 and descriptions thereof).

In some embodiments, after the orientation of the target subject is determined, the processing device 120 may process the image data based on the orientation of the target subject, and cause a terminal device of the user to display the processed image data. For example, if the orientation of the target subject is different from a reference orientation (e.g., a head-up orientation), the image data may be rotated to generate the processed image data, wherein a representation of the target subject in the processed image data may have the reference orientation. In some embodiments, the processing device 120 may process another set of image data (e.g., a medical image acquired by the medical imaging device 110) based on the orientation of the target subject. In some embodiments, operation 608 may be performed after the scan of the target subject to determine the orientation of the target subject based on medical image data acquired in the scan.

In 609, the processing device 120 (e.g., the analyzing module 720) may perform a preparation check. Operation 609 may be performed in a similar manner with operation 511 as described in connection with FIG. 5, and the descriptions thereof are not repeated here.

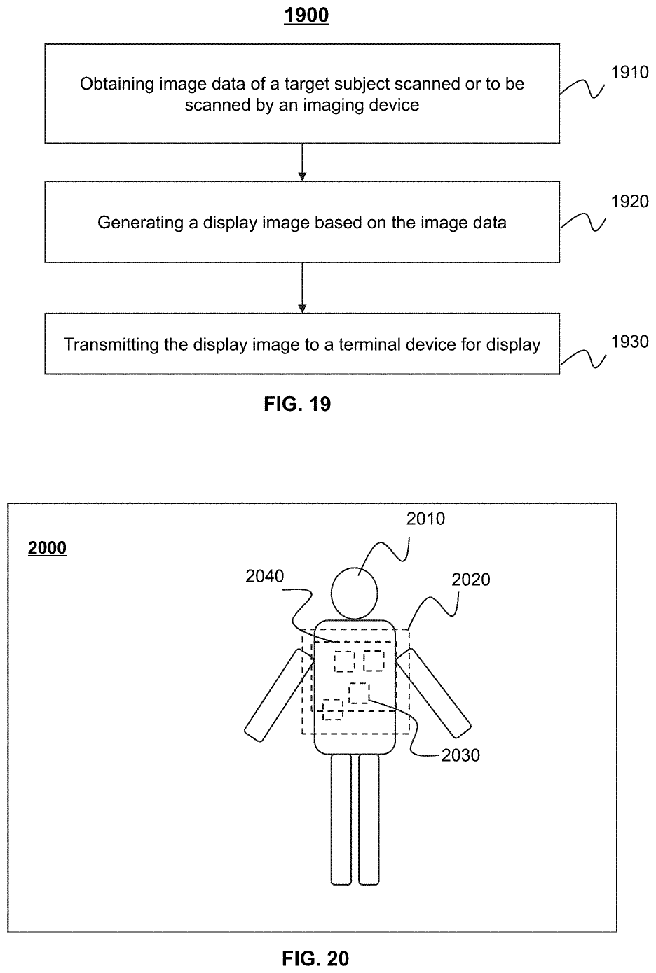

In some embodiments, as shown in FIG. 6, a collision detection may be performed during the implementation of the process 600 (or a portion thereof). For example, the processing device 120 may obtain real-time image data of the examination room, and track the movement of components (e.g., a human, the image capturing device) in the examination room based on the real-time image data. The processing device 120 may further estimate the likelihood of a collision between two or more components in the examination room. If it is detected that a collision between different components is likely to occur, the processing device 120 may cause a terminal device to output a notification regarding the collision. Additionally or alternatively, a visual interactive interface may be used to achieve a user interaction between the user and the imaging system and/or between the target subject and the imaging system. The visual interactive interface may be implemented on, for example, a terminal device 140 as described in connection with FIG. 1 or a mobile device 300 as described in connection with FIG. 3. The visual interactive interface may present data obtained and/or generated by the processing device 120 (e.g., an analysis result, an intermediate result) in the implementation of the process 600. For example, one or more display images as described in connection with FIG. 19 may be displayed by the visual interactive interface. Additionally or alternatively, the visual interactive interface may receive a user input from the user and/or the target subject.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations of the process 500B and the process 600 may be added or omitted. For example, one or more of the operations 601, 608, and 609 may be omitted. In some embodiments, two or more operations may be performed simultaneously. For example, operation 601 and operation 602 may be performed simultaneously. As another example, operation 602 and operation 603 may be performed simultaneously. As yet another example, operation 605 may be performed before operation 604. In some embodiments, an automatic preparation operation of the process 500B or the process 600 may be performed by the processing device 120 semi-automatically based on user intervention or manually by a user.

FIG. 7 is a block diagram illustrating an exemplary processing device 120 according to some embodiments of the present disclosure. As shown in FIG. 7, the processing device 120 may include an acquisition module 710, an analyzing module 720, and a control module 730.

The acquisition module 710 may be configured to obtain information relating to the imaging system 100. For example, the acquisition module 710 may obtain image data of a target subject before, during, and/or after the target subject is scanned by a medical imaging device, wherein the image data may be captured by an image capturing device (e.g., a camera mounted in an examination room where the target subject is located). As another example, the acquisition module 710 may obtain reference information including, such as reference identity information, reference feature information, reference image data of the target subject. As yet another example, the acquisition module 710 may obtain a reference posture model of the target subject. As yet another example, the acquisition module 710 may obtain at least one parameter value of at least one scanning parameter relating to a scan to be performed on the target subject.

The analyzing module 720 may be configured to perform one or more scan preparation operations for a scan of the target subject by analyzing the information obtained by the acquisition module 710. More descriptions regarding the analysis of the information and the scan preparation operation(s) may be found elsewhere in the present disclosure. See, e.g., FIG. 6 and FIGS. 8-21 and relevant descriptions thereof.

The control module 730 may be configured to control one or more components of the imaging system 100. For example, the control module 730 may cause movable component(s) of the medical imaging device to move to their respective target position(s). More descriptions of the determination of the target position of a movable component of the medical imaging device may be found elsewhere in the present disclosure (e.g., FIGS. 10, 11A, 11B, 21, and descriptions thereof).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 120 may further include a storage module (not shown in FIG. 7). The storage module may be configured to store data generated during any process performed by any component of in the processing device 120. As another example, each of components of the processing device 120 may include a storage device. Additionally or alternatively, the components of the processing device 120 may share a common storage device.

FIG. 8 is a flowchart illustrating an exemplary process for identfiying a target subject to be scanned according to some embodiments of the present disclosure. In some embodiments, the process 800 may be implemented in the imaging system 100 illustrated in FIG. 1. For example, the process 800 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 800 may be accomplished with one or more additional operations not described, and/or without one or more of the operations discussed. Additionally, the order in which the operations of the process 800 as illustrated in FIG. 8 and described below is not intended to be limiting.

In 810, the processing device 120 (e.g., the acquisition module 710) may obtain image data of one or more candidate subjects. The image data may be captured by a first image capturing device when or after the candidate subject(s) enter an examination room.

In some embodiments, the one or more candidate subjects may include the target subject to be examined. For example, the target subject may be a patient to be imaged by a medical imaging device (e.g., the medical imaging device 110) in the examination room. In some embodiments, the one or more candidate subjects may further include one or more subjects other than the target subject. For example, the candidate subject(s) may include a companion (e.g., a relative, a friend) of the target subject, a doctor, a nurse, a technician, or the like.

As used herein, image data of a target subject (e.g., a candidate subject, a target subject) refers to image data corresponding to the entire subject or image data corresponding to a portion of the target subject (e.g., a body part including a face of a patient). In some embodiments, the image data of the target subject may include a two-dimensional (2D) image, a three-dimensional (3D) image, a four-dimensional (4D) image (e.g., a series of images over time), and/or any related image data (e.g., scan data, projection data). In some embodiments, the image data of the candidate subject(s) may include color image data, point-cloud data, depth image data, mesh data, or the like, or any combination thereof, of the candidate subject(s).

The image data of the candidate subject(s) may be captured by the first image capturing device (e.g., the image capturing device 160) mounted in the examination room or at the door of the examination room. The first image capturing device may include any type of device that is capable of acquiring image data as described elsewhere in this disclosure (e.g., FIG. 1 and the relevant descriptions), such as a 3D camera, an RGB sensor, an RGB-D sensor, a 3D scanner, a 3D laser imaging device, a structured light scanner, or the like. In some embodiments, the first image capturing device may automatically capture the image data of the one or more candidate subjects when or after the one or more candidate subjects enter the examination room.

In some embodiments, the processing device 120 may obtain the image data from the first image capturing device. Alternatively, the image data may be acquired by the first image capturing device and stored in a storage device (e.g., the storage device 130, the storage device 220, the storage 390, or an external source). The processing device 120 may retrieve the image data from the storage device.

In 820, the processing device 120 (e.g., the acquisition module 710) may obtain reference information associated with the target subject to be examined.

The reference information associated with the target subject may include reference image data of the target subject, reference identity information of the target subject, one or more reference features of the target subject, or any other information that may be used to distinguish the target subject from other subjects, or any combination thereof. The reference image data of the target subject may include image data that includes the human face of the target subject. For example, the reference image data may include an image of the target subject after the identity of the target subject is confirmed. The reference identify information may include an identification (ID) number, a name, the gender, the age, a date of birth, an occupation, contact information (e.g., a mobile phone number), a driver's license, or the like, or any combination thereof, of the target subject. The one or more reference features may include a body shape (e.g., a contour, a height, a width, a thickness, a ratio between two dimensions of the body), clothing (e.g., color, style), or the like, or any combination thereof, of the target subject.

In some embodiments, the reference information of the target subject may be obtained by, for example, one or more image capturing devices on the spot in or out of the examination room. Additionally or alternatively, the reference information of the target subject may be previously generated and stored in a storage device (e.g., the storage device 130, the storage device 220, the storage 390, or an external source). The processing device 120 may retrieve the reference information from the storage device.

Taking the reference image data of the target subject as an example, it may be captured by a second image capturing device that is mounted in or outside the examination room. The first image capturing device and the second image capturing device may be of a same type or different types. In some embodiments, the second image capturing device may be the same device as the first image capturing device. Merely by way of example, before, when, or after a subject enters the examination room, a quick response (QR) code on his/her medical card or examination application form may be scanned by a scanner (e.g., a component of the second image capturing device) in order to confirm the identity of the subject. If it is confirmed that the subject is the target subject, the second image capturing device may be directed to capture the reference image data of the target subject.

As another example, the target subject may be instructed to make a specific behavior (e.g., make a specific gesture and/or sound, stand in a specific area for a period of time that exceeds a time threshold) before, when, or after he/she enters the examination room. The processing device 120 may be configured to track the state (e.g., a gesture, a posture, an expression, a sound) of each candidate subject based on, for example, image data captured by the second image capturing device. If a certain candidate subject makes the specific behavior, the candidate subject may be determined as the target subject, and the second image capturing device may capture the image data of the certain candidate subject as the reference image data.

In some embodiments, the reference information of the target subject may be obtained based on a replication image of an identification certification of the target subject. The identification certification may be an identity card, a medical insurance card, a medical card, an examination application form, or the like, of the target subject. For example, the replication image of the identification certification may be obtained by an image capturing device (e.g., the first image capturing device, the second image capturing device, another image capturing device) via scanning the identification certification before, when, or after the target subject enters the examination room. As another example, the replication image of the identification certification may be previously generated and stored in a storage device, such as a storage device of the imaging system 100 or another system (e.g., a public security system). The processing device 120 may obtain the replication image from the image capturing device or the storage device, and determine the reference information of the target subject based on the replication image.

For example, the identify certification may include an identification photo of the target subject. The processing device 120 may detect a human face of the target subject in the replication image according to one or more face detection algorithms. Exemplary face detection or recognition algorithms may include a knowledge-based technique, a feature-based technique, a template matching technique, an eigenface-based technique, a distribution-based technique, a neural-network based technique, a support vector machine (SVM) based technique, a sparse network of winnows (SNoVV) based technique, a naive bayes classifier, a hidden markov model, an information theoretical algorithm, an inductive learning technique, or the like. The processing device 120 may segment the human face of the target subject from the replication image based on one or more image segmentation algorithms. Exemplary image segmentation algorithms may include a region-based algorithm (e.g., a threshold segmentation, a region-growth segmentation), an edge detection segmentation algorithm, a compression-based algorithm, a histogram-based algorithm, a dual clustering algorithm, or the like. The segmented human face of the target subject may be designated as the reference image data of the target subject.

As another example, the identification certification may include the reference identity information of the target subject. The processing device 120 may recognize the reference identity information in the replication image according to one or more text recognition algorithms. Exemplary text recognition algorithms may include a template algorithm, an indicative algorithm, a structural recognition algorithm, an artificial neural network, or the like.

In some embodiments, the reference information of the target subject may be determined based on a unique symbol associated with the target subject. The unique symbol may include a bar code, a QR code, a serial number including letters and/or digits, or the like, or any combination thereof. For example, the reference information of the target subject may be obtained by scanning the QR code on a wristband or a sticker of the target subject via an image capturing device (e.g., the first image capturing device, the second image capturing device, or another image capturing device). In some embodiments, a user, e.g., the target subject or a doctor may manually input the reference identity information via a terminal device (e.g., the terminal device 140) of the imaging system 100.

In 830, the processing device 120 (e.g., the analyzing module 720) may identify, from the one or more candidate subjects, the target subject based on the reference information and the image data.

In some embodiments, the processing device 120 may identify the target subject from the one or more candidate subjects based on the reference image data of the target subject and the image data of the one or more candidate subjects. Merely by way of example, the processing device 120 may extract reference feature information of the target subject from the reference image data. The reference feature information may include a shape (e.g., a contour, an area, a height, a width, a ratio of height to width), a color, a texture, or the like, or any combination thereof, of the target subject or a portion of the target subject, such as a face component (e.g., eyes, the nose, the mouth) of the target subject. For example, the processing device 120 may detect a human face of the target subject in the reference image data according to one or more face detection algorithms as described elsewhere in the present disclosure. The processing device 120 may extract the feature information of the human face of the target subject according to one or more feature extraction algorithms. Exemplary feature extraction algorithms may include a principal component analysis (PCA), a linear discriminant analysis (LDA), an independent component analysis (ICA), a multi-dimensional scaling (MDS) algorithm, a discrete cosine transform (DCT) algorithm, or the like, or any combination thereof. The processing device 120 may further extract feature information of each of the one or more candidate subjects from the image data. The extraction of the feature formation of the each candidate subject from the image data may be performed in a similar manner as that of the reference feature information of the target subject from the reference image data.

The processing device 120 may then identify the target subject based on the reference feature information of the target subject and the feature information of the each of the one or more candidate subjects. For example, for the each candidate subject, the processing device 120 may determine a degree of similarity between the target subject and the candidate subject based on the reference feature information of the target subject and the feature information of the candidate subject. The processing device 120 may further select, among the candidate subject(s), a candidate subject that has the highest degree of similarity to the target subject as the target subject.

The degree of similarity between the target subject and a candidate subject may be determined by various approaches. Merely by way of example, the processing device 120 may determine a first feature vector representing the reference feature information of the target subject (also referred to as the first feature vector corresponding to the target subject). The processing device 120 may determine a second feature vector representing the feature information of the candidate subject (also referred to as the second feature vector corresponding to the candidate subject). The processing device 120 may determine the degree of similarity between the target subject and the candidate subject by determining a degree of similarity between the first feature vector and the second feature vector. A degree of similarity between two feature vectors may be determined based on a similarity algorithm, e.g., a Euclidean distance algorithm, a Manhattan distance algorithm, a Minkowski distance algorithm, a cosine similarity algorithm, a Jaccard similarity algorithm, a Pearson correlation algorithm, or the like, or any combination thereof.

In some embodiments, the processing device 120 may identify the target subject from the one or more candidate subjects based on the reference identity information of the target subject and identify information of the each candidate subject of the one or more candidate subjects. For example, for the each candidate subject, the processing device 120 may determine the identity information of the candidate subject based on the image data. In some embodiments, the processing device 120 may segment a human face of each candidate subject from the image data according to, for example, one or more face detection algorithms and/or one or more image segmentation algorithms as described elsewhere in the present disclosure.

For each candidate subject, the processing device 120 may then determine the identity information of the candidate subject based on the human face of the candidate subject and an identity information database. Exemplary identity information databases may include a public security database, a medical insurance database, a social insurance database, or the like. The identity information database may store a plurality of human faces of a plurality of subjects (humans) and their respective identity information. For example, the processing device 120 may determine a degree of similarity between the human face of the candidate subject and each human face stored in the identity information database, and select a target human face that has the highest degree of similarity to the human face of the candidate subject identified. In some embodiments, a degree of similarity between a human face of a candidate subject and a human face stored in the identity information database may be determined based on a degree of similarity between a feature vector representing feature information of the human face of the candidate subject and a feature vector representing feature information of the human face stored in the identity information database. The processing device 120 may determine identity information corresponding to the selected target human face as the identity information of the candidate subject. The processing device 120 may further identify the target subject from the at least one candidate subject by comparing the identity information of the each candidate subject with the reference identity information of the target subject. For example, the processing device 120 may compare an ID number of the each candidate subject and a reference ID number of the target subject. The processing device 120 may determine a candidate subject having a same ID number as the reference ID number as the target subject.

In some embodiments, the processing device 120 may identify the target subject from the one or more candidate subjects based on a combination of the reference image data and the reference identity information of the target subject. For example, the processing device 120 may determine a first target subject from the at least one candidate subject based on the reference image data of the target subject and the image data of the one or more candidate subjects. The processing device 120 may determine a second target subject from the one or more candidate subjects based on the reference identity information of the target subject and the identity information of the each of the one or more candidate subjects. The processing device 120 may determine whether the first target subject is the same as the second target subject. If the first target subject is the same as the second target subject, the processing device 120 may determine that the first target subject (or the second target subject) is the final target subject. In such cases, the accuracy of the identification of the target subject may be improved.

If the first target subject is different from the second target subject, the processing device 120 may re-identify the first and second target subjects and/or generate a reminder regarding the identification result. The reminder may be in the form of text, voice, an image, a video, a haptic alert, or the like, or any combination thereof. For example, the processing device 120 may transmit the reminder to a terminal device (e.g., the terminal device 140) of a user (e.g., a doctor) of the imaging system 100. The terminal device may output the reminder to the user. Optionally, the user may input an instruction or information in response to the reminder. Merely by way of example, the user may manually select the final target subject from the first target subject and the second target subject. For example, the processing device 120 may cause the terminal device to display information (e.g., image data, identity information) of the first target subject and the second target subject. The user may select the final target subject from the first target subject and the second target subject based on the information of the first target subject and the second target subject.

In some embodiments, the processing device 120 may identify the target subject from the one or more candidate subjects based on one or more reference features of the target subject and the image data of the one or more candidate subjects. For example, the processing device 120 may detect each candidate subject in the image data and further extract one or more features of the candidate subject. The processing device 120 may identify the target subject from the one or more candidate subjects by comparing the one or more features of the each candidate subject with the one or more reference features of the target subject. Merely by way of example, the processing device 120 may select a candidate subject having the most similar body shape to the target subject as the target subject.

Based on the image data of the candidate subject(s) and the reference information of the target subject, the target subject may be identified from the candidate subject(s) automatically. Compared with conventional imaging procedures in which a user (e.g., a doctor or nurse) needs to determine the target subject and check the identification of the target subject by, for example, looking up profile information of the target subject (e.g., by visually inspecting the candidate subject(s) with respect to a profile image of the target subject), the target subject identification methods disclosed herein may obviate the need of subjective judgment and be more efficient and accurate.

In some embodiments, after the image data of the one or more candidate subjects is obtained, the processing device 120 may cause the terminal device (e.g., the terminal device 140) of the user to display the image data. The processing device 120 may obtain, via the terminal device, an input associated with the target subject from the user. The processing device 120 may identify the target subject from the one or more candidate subjects based on the input. For example, the terminal device may display the image data, and the user may select (e.g., by clicking an icon corresponding to) a specific candidate subject from the displayed image via an input component of the terminal device (e.g., a mouse, a touch screen). The processing device 120 may determine the selected candidate subject as the target subject.

In some embodiments, after the target subject (or the final target subject) is identified, the processing device 120 may perform one or more additional operations to prepare for the scan of the target subject. For example, the processing device 120 may generate a target posture model of the target subject. As another example, the processing device 120 may cause movable component(s) (e.g., a scanning table) of a medical imaging device to move to their respective target positions. As yet another example, the processing device 120 may determine value(s) of scanning parameter(s) (e.g., a light field) corresponding to the target subject. More descriptions regarding the preparation of the scan may be found elsewhere in the present disclosure. See, e.g., FIG. 6 and relevant descriptions thereof.

Compared to a conventional way that a user needs to manually identify the target subject and/or check the identity of the target subject, the automated target subject identification systems and methods disclosed herein may be more accurate and efficient by, e.g., reducing the workload of a user, cross-user variations, and the time needed for the target subject identification.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a process for preprocessing (e.g., denoising) the image data of the at least one candidate subject may be added before operation 830. In some embodiments, two or more operations may be performed simultaneously. For example, operation 810 and operation 820 may be performed simultaneously. As another example, operation 820 may be performed before operation 810.

FIG. 9 is a flowchart illustrating an exemplary process for generating a target posture model of a target subject according to some embodiments of the present disclosure. In some embodiments, the process 900 may be implemented in the imaging system 100 illustrated in FIG. 1. For example, the process 900 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 900 may be accomplished with one or more additional operations not described, and/or without one or more of the operations discussed. Additionally, the order in which the operations of the process 900 as illustrated in FIG. 9 and described below is not intended to be limiting.

In 910, the processing device 120 (e.g., the acquisition module 710) may obtain image data of a target subject (e.g., a patient) to be examined (or scanned).

The image data may include a 2D image, a 3D image, a 4D image (e.g., a time series of 3D images), and/or any related image data (e.g., scan data, projection data) of the target subject. The image data may include color image data, point-cloud data, depth image data, mesh data, medical image data, or the like, or any combination thereof, of the target subject.

In some embodiments, the image data of the target subject may be captured by an image capturing device, such as the image capturing device 160, mounted in an examination room. The image capturing device may include any type of device that is capable of acquiring image data, such as a 3D camera, an RGB sensor, an RGB-D sensor, a 3D scanner, a 3D laser imaging device, a structured light scanner. In some embodiments, the image capturing device may obtain the image data of the target subject before the target subject is positioned at a scan position. For example, the image data of the target subject may be captured after the target subject enters the examination room and the identity of the target subject is confirmed (e.g., after the process 800 as described in connection with FIG. 8 is implemented).

In some embodiments, the processing device 120 may obtain the image data of the target subject from the image capturing device. Alternatively, the image data may be acquired by the image capturing device and stored in a storage device (e.g., the storage device 130, the storage device 220, the storage 390, or an external source). The processing device 120 may retrieve the image data from the storage device.

In 920, the processing device 120 (e.g., the analyzing module 720) may generate a subject model of the target subject based on the image data.

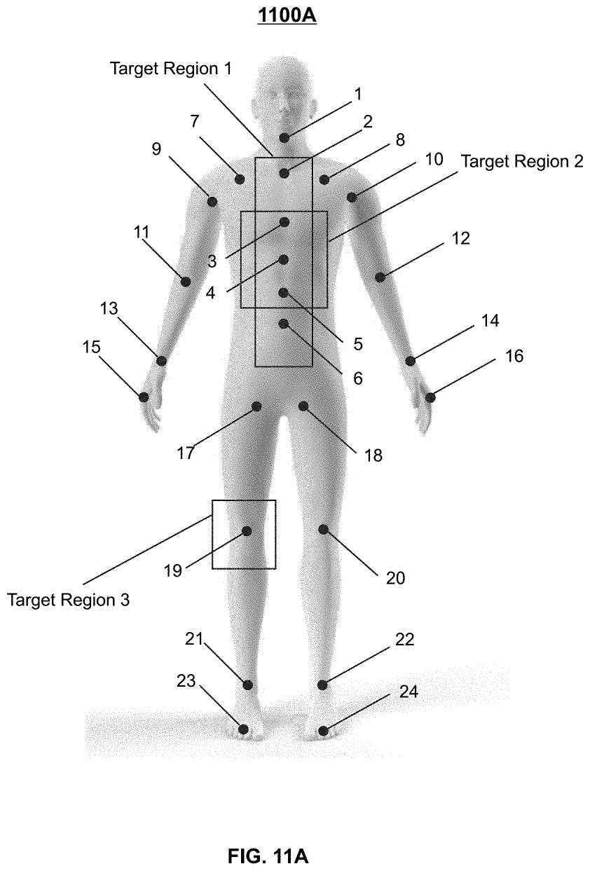

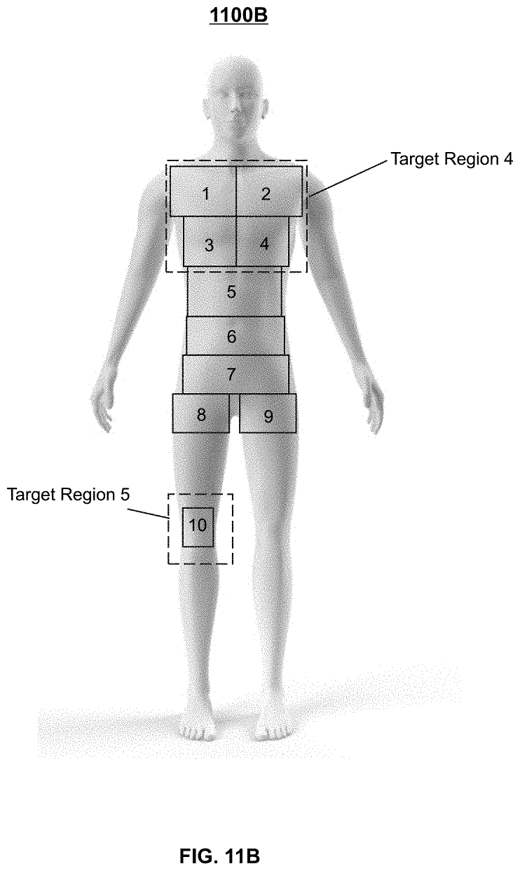

As used herein, a subject model of a target subject (e.g., a subject model 1100 as illustrated in FIG. 11) determined based on image data of the target subject refers to a model representing the target subject holding a posture when the image data is captured. A posture of a target subject may reflect one or more of a position, a pose, a shape, a size, etc., of the target subject (or a portion thereof).

In some embodiments, the subject model may include a 2D skeleton model, a 3D skeleton model, a 3D mesh model, or the like. A 2D skeleton model of a target subject may include an image illustrating one or more anatomical joints and/or bones of the target subject in 2D space. A 3D skeleton model of a target subject may include an image illustrating one or more anatomical joints and/or bones of the target subject in 3D space. A 3D mesh model of a target subject may include a plurality of vertices, edges, and faces that define a 3D shape of the target subject.

In some embodiments, the processing device 120 may generate the subject model of the target subject based on the image data of the target subject. For illustration purposes, an exemplary generation process of a 3D mesh model of the target subject is described hereinafter as an example. The processing device 120 may extract body surface data of the target subject (or a portion thereof) from the image data by, for example, performing an image segmentation operation on the image data according to one or more image segmentation algorithms as described elsewhere in the present disclosure. The body surface data may include a plurality of pixels (or voxels) corresponding to a plurality of physical points of the body surface of the target subject. In some embodiments, the body surface data may be represented in a mask, which includes a two-dimensional matrix array, a multi-value image, or the like, or any combination thereof. In some embodiments, the processing device 120 may process the body surface data. For example, the processing device 120 may remove a plurality of noise points (e.g., a plurality of pixels of clothes or accessories) from the body surface data. As another example, the processing device 120 may perform a filtering operation, a smoothing operation, a boundary calculation operation, or the like, or any combination thereof, on the body surface data. The processing device 120 may further generate the 3D mesh model based on the (processed) body surface data. For example, the processing device 120 may generate a plurality of meshes by combining (e.g., connecting) a plurality of points of the body surface data.

In some embodiment, the processing device 120 may generate the 3D mesh model of the target subject based on the image data according to one or more mesh generation techniques, such as a Triangular/Tetrahedral (Tri/Tet) technique (e.g., an Octree algorithm, an Advancing Front algorithm, a Delaunay algorithm, etc.), a Quadrilateral/Hexahedra (Quad/Hex) technique (e.g., a Trans-finite Interpolation (TFI) algorithm, an Elliptic algorithm, etc.), a hybrid technique, a parametric model based technique, a surface meshing technique, or the like, or any combination thereof.

In some embodiments, one or more feature points may be identified from the subject model. For example, a feature point may correspond to a specific physical point of the target subject, such as an anatomical joint (e.g., a shoulder joint, a knee joint, an elbow joint, an ankle joint, a wrist joint) or another representative physical point in a body region (e.g., the head, the neck, a hand, a leg, a foot, a spine, a pelvis, a hip) of the target subject.

In some embodiments, the one or more feature points may be annotated manually by a user (e.g., a doctor, an imaging specialist, a technician) on an interface (e.g., implemented on a terminal device 140) that displays the image data. Alternatively, the one or more feature points may be generated by a computing device (e.g., the processing device 120) automatically according to an image analysis algorithm (e.g., an image segmentation algorithm, a feature point extraction algorithm). Alternatively, the one or more feature points may be generated by the computing device semi-automatically based on an image analysis algorithm in combination with information provided by a user. Exemplary information provided by the user may include a parameter relating to the image analysis algorithm, a position parameter relating to a feature point, an adjustment to, or rejection or confirmation of a preliminary feature point generated by the computing device, etc.

In some embodiments, the subject model may be represented by one or more model parameters, such as one or more contour parameters and/or one or more posture parameters of the subject model or the target subject represented by the subject model. For example, the one or more contour parameters may be a quantitative expression that describes the contour of the subject model (or the target subject). Exemplary contour parameters may include a shape and/or a size (e.g., a height, a width, a thickness) of the subject model or a portion of the subject model. The one or more posture parameters may be a quantitative expression that describes the posture of the subject model (or the target subject). Exemplary posture parameters may include a position of a feature point of the reference posture model (e.g., a coordinate of a joint in a certain coordinate system), a relative position between two feature points of the reference posture model (e.g., a joint angle of a joint), or the like.

In 930, the processing device 120 (e.g., the acquisition module 710) may obtain a reference posture model associated with the target subject.

As used herein, a reference posture model refers to a model representing a reference subject holding a reference posture. The reference subject may be a real human or a phantom. The reference posture model may include a 2D skeleton model, a 3D skeleton model, a 3D mesh model, or the like, of the reference subject. In some embodiments, the reference posture model may be represented by one or more model parameters, such as one or more reference contour parameters and/or one or more reference posture parameters of the reference posture model or the reference subject represented by the reference posture model. The one or more reference contour parameters may be a quantitative expression that describes the contour of the reference posture model or the reference subject. The one or more reference posture parameters may be a quantitative expression that describes the posture of the reference posture model or the reference subject. Exemplary reference contour parameters may include a shape and/or a size (e.g., a height, a width, a thickness) of the reference posture model or a portion of the reference posture model. Exemplary reference posture parameters may include a position of a reference feature point of the reference posture model (e.g., a coordinate of a joint in a certain coordinate system), a relative position between two reference feature points of the reference posture model (e.g., a joint angle of a joint), or the like.

In some embodiments, the reference posture model and the subject model may be of a same type of model or different types of models. For example, the reference posture model and the subject model may be 3D mesh models. As another example, the subject model may be represented by a plurality of model parameters (e.g., one or more contour parameters and one or more posture parameters), and the reference posture model may be a 3D mesh model. The reference posture may be a standard posture that the target subject needs to hold during the scan to be performed on the target subject. Exemplary reference postures may include a head-first supine posture, a feet-first prone posture, a head-first left lateral recumbent posture, or a feet-first right lateral recumbent posture, or the like.

In some embodiments, the processing device 120 may obtain the reference posture model associated with the target subject based on an imaging protocol of the target subject. The imaging protocol may include, for example, value(s) or value range(s) of one or more scanning parameters (e.g., an X-ray tube voltage and/or current, an X-ray tube angle, a scan mode, a table moving speed, a gantry rotation speed, a field of view (FOV)), a source image distance (SID), a portion of the target subject to be imaged, feature information of the target subject (e.g., the gender, the body shape), or the like, or any combination thereof. The imaging protocol (or a portion thereof) may be determined manually by a user (e.g., a doctor) or by one or more components (e.g., the processing device 120) of the imaging system 100 according to different situations.

For example, the imaging protocol may define the portion of the target subject to be imaged, and the processing device 120 may obtain the reference posture model corresponding to the portion of the target subject to be imaged. Merely by way of example, if the chest of the target subject needs to be imaged, a first reference posture model corresponding to a chest examination may be obtained. The first reference posture model may represent a reference subject who is standing on the floor and placing his/her hands on the waist. As another example, if the vertebral of the target subject needs to be imaged, a second reference posture model corresponding to a vertebral examination may be obtained. The second reference posture model may represent a reference subject who lies on a scanning table with legs and arms splaying on the scanning table.

In some embodiments, a posture model library having a plurality of posture models may be previously generated and stored in a storage device (e.g., the storage device 130, the storage device 220, and/or the storage 390, an external source). In some embodiments, the posture model library may be updated from time to time, e.g., periodically or not, based on data of reference subjects that are at least partially different from original data from which an original posture model library is generated. The data of a reference subject may include a portion of the reference subject to be imaged, one or more features (e.g., the gender, the body shape) of the reference subject, or the like. In some embodiments, the plurality of posture models may include posture models corresponding to different examination regions of human. For example, for each examination region (e.g., the chest, the vertebral, the elbow), a set of posture models may be available, wherein each posture model in the set may represent a reference subject who has a particular feature (e.g., having a particular gender, and/or a particular body shape) and hold a reference posture corresponding to the examination region. Merely by way of example, for the chest of human, the corresponding set of posture models may include posture models representing a plurality of reference subjects who hold a standard posture for the chest examination and have different body shapes (e.g., heights and/or weights).

The posture models (or a portion thereof) may be previously generated by a computing device (e.g., the processing device 120) of the imaging system 100. Additionally or alternatively, the posture models (or a portion thereof) may be generated and provided by a system of a vendor that provides and/or maintains such posture models, wherein the system of the vendor is different from the imaging system 100. The processing device 120 may generate or retrieve the posture models from the computing device and/or a storage device that stores the posture models directly or via a network (e.g., the network 150).

The processing device 120 may further select the reference posture model from the posture model library based on the portion of the target subject to be imaged and one or more features (e.g., the gender, the body shape, or the like) of the target subject. For example, the processing device 120 may acquire a set of posture models corresponding to the portion of the target subject to be imaged, and select one from the set of posture models as the reference posture model. The selected posture model may represent a reference subject having the same feature as or a similar feature to the target subject. Merely by way of example, if the portion of the target subject to be imaged is the chest and the target subject is a female, the processing device 120 may obtain a set of posture models corresponding to the chest examination, and select a posture model that represents a female reference subject as the reference posture model of the target subject. By generating the posture models in advance, the generation process of the reference posture model may be simplified, which in turn, may improve the efficiency of the generation of the target posture model of the target subject.

In some embodiments, the reference posture model of the reference subject may be annotated with one or more reference feature points. Similar to a feature point of the subject model, a reference feature point may correspond to a specific anatomical point (e.g., a joint) of the reference subject. The identification of the reference feature point(s) from the reference posture model may be performed in a similar manner with that of the feature point(s) from the subject model as described in connection with operation 920, and the descriptions thereof are not repeated here.

In 940, the processing device 120 (e.g., the analyzing module 720) may generate the target posture model of the target subject based on the subject model and the reference posture model. As used herein, a target posture model of the target subject refers to a model representing the target subject holding the reference posture.

In some embodiments, the processing device 120 may generate the target posture model of the target subject by transforming the subject model according to the reference posture model. For example, the processing device 120 may obtain one or more reference posture parameters of the reference posture model. The one or more reference posture parameters may be previously generated by a computing device and stored in a storage device, such as a storage device (e.g., the storage device 130) of the imaging system 100. Alternatively, the one or more reference posture parameters may be determined by the processing device 120 by analyzing the reference posture model.

The processing device 120 may further generate the target posture model of the target subject by transforming the subject model based on the one or more reference posture parameters. In some embodiments, the processing device 120 may perform one or more image processing operations (e.g., rotation, translation, distortion) on one or more portions of the subject model based on the one or more reference posture parameters, so as to the generate the target posture model. For example, the processing device 120 may rotate a portion of the subject model representing the right wrist of the target subject so that the joint angle of the right wrist of the target subject in the transformed subject model may be equal to or substantially equal to a reference value of the joint angle of the right wrist of the reference posture model. As another example, the processing device 120 may translate a first portion representing the left ankle of the target subject and/or a second portion representing the right ankle of the target subject so that the distance between the first and second portions in the transformed subject model may be equal to or substantially equal to the distance between the left ankle and the right ankle of the reference posture model.

In some embodiments, the processing device 120 may generate the target posture model of the target subject by transforming the reference posture model according to the subject model. For example, the processing device 120 may obtain one or more contour parameters of the subject model. The one or more contour parameters may be previously generated by a computing device and stored in a storage device, such as a storage device (e.g., the storage device 130) of the imaging system 100. Alternatively, the one or more contour parameters may be determined by the processing device 120 by analyzing the subject model.

The processing device 120 may further generate the target posture model of the target subject by transforming the reference posture model based on the one or more contour parameters of the subject model. In some embodiments, the processing device 120 may perform one or more image processing operations (e.g., rotation, translation, distortion) on one or more portions of the reference posture model based on the one or more contour parameters, so as to the generate the target posture model. For example, the processing device 120 may stretch or shrink the reference posture model so that the height of the transformed reference posture model may be equal to or substantially equal to the height of the subject model.

In some embodiments, the subject model and/or the target posture model may be utilized in one or more other scan preparation operations by the processing device 120. For example, the processing device 120 may cause movable component(s) (e.g., a scanning table) of a medical imaging device to move to their respective target positions based on the subject model. In some embodiments, the target posture model may be used to assist the positioning of the target subject. For example, the target posture model or a composite image generated based on the target posture model may be displayed to the target subject to guide the target subject to adjust his/her posture. As another example, after the target subject is positioned to a scan position, the processing device 120 may determine whether a posture of the target subject needs to be adjusted based on the target posture model. Compared with conventional positioning approaches which need a user (e.g., a doctor) to check the posture of the target subject and/or instruct the target subject to adjust his/her posture, the target subject positioning technique disclosed herein may be implemented without or with reduced or minimal user intervention, which is time-saving, more efficient, and more accurate. More descriptions regarding the utilization of the subject model and/or the target posture model may be found elsewhere in the present disclosure. See, e.g., FIGS. 16A to 17 and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a process for preprocessing (e.g., denoising) the image data of the target subject may be added before operation 920. In some embodiments, two or more operations may be performed simultaneously. For example, operation 920 and operation 930 may be performed simultaneously. As another example, operation 930 may be performed before operation 920.

FIG. 10 is a flowchart illustrating an exemplary process for scan preparation according to some embodiments of the present disclosure. In some embodiments, the process 1000 may be implemented in the imaging system 100 illustrated in FIG. 1. For example, the process 1000 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1000 may be accomplished with one or more additional operations not described, and/or without one or more of the operations discussed. Additionally, the order in which the operations of the process 1000 as illustrated in FIG. 10 and described below is not intended to be limiting.

In 1010, the processing device 120 (e.g., the acquisition module 710) may obtain image data of a target subject.

Operation 1010 may be performed in a similar manner as operation 910 as described in connection with FIG. 9, and the descriptions thereof are not repeated here.

In 1020, for one or more movable components of a medical imaging device, the processing device 120 (e.g., the analyzing module 720) may determine, based on the image data, a target position of each of the one or more movable components of the medical imaging device.

The medical imaging device may be used to perform a scan on the target subject. In some embodiments, the medical imaging device (e.g., the medical imaging device 110) may be an X-ray imaging device (e.g., a suspended X-ray imaging device, a C-arm X-ray imaging device), a digital radiography (DR) device (e.g., a mobile digital X-ray imaging device), a CT device, a PET device, an MRI device, or the like. Merely by way of example, for an X-ray imaging device, the one or more movable components of the X-ray imaging device may include a scanning table (e.g., the scanning table 114), a detector (e.g., the detector 112, the flat panel detector 440), an X-ray source (e.g., the radiation source 115, the X-ray source 420), or the like. A target position of a movable component refers to an estimated position where the movable component needs to be located during the scan of the target subject according to, for example, the posture of the target subject and/or an imaging protocol of the target subject.

In some embodiments, the processing device 120 may determine a target position of a movable component (e.g., a scanning table) by determining a height of the target subject based on the image data. For example, the processing device 120 may identify a representation of the target subject in the image data, and determine a reference height of the representation of the target subject in the image domain. Merely for illustration purposes, a first point at the feet of the target subject and a second point at the top of the head of the target subject may be identified in the image data. A pixel distance (or voxel distance) between the first point and the second point may be determined as the reference height of the representation of the target subject in the image domain. The processing device 120 may then determine the height of the target subject in the physical world based on the reference height and one or more parameters (e.g., intrinsic parameters, extrinsic parameters) of the image capturing device that captures the image data.

The processing device 120 may further determine the target position (e.g., a height) of the movable component based on the height of the target subject. For example, the processing device 120 may determine the height of the scanning table as ⅓, ½, or the like, of the height of the target subject. The height of the scanning table may be represented as, for example, a Z-axis coordinate of the surface of the scanning table where the target subject lies on in the coordinate system 470 as shown in FIG. 4A. In this way, the height of the scanning table may be determined and adjusted automatically based on the height of the target subject, which may be convenient for the target subject to get on and/or get off the scanning table. After the target subject gets on the scanning table, the scanning table may further move to a second target position to get ready for the target subject to be imaged (or treated).

Additionally or alternatively, the processing device 120 may determine a target position of a movable component by generating a subject model (or a target posture model as described in FIG. 9) based on the image data of the target subject. More descriptions of the generation of the subject model (or the target posture model) may be found elsewhere in the present disclosure (e.g., FIG. 9 and descriptions thereof). The processing device 120 may determine a target region in the subject model, wherein the target region may correspond to an ROI of the target subject. An ROI may include one or more physical portions (e.g., a tissue, an organ) of the target subject to be imaged by the medical imaging device. The processing device 120 may further determine the target position of the movable component based on the target region.

For illustration purposes, the determination of the target position of a detector (e.g., a flat panel detector) of the medical imaging device based on the target region is described as an example. In some embodiments, based on the target region, the processing device 120 may determine the target position of the detector at which the detector may cover the entire ROI of the target subject when the target subject is located at a scan position. In such cases, the detector may receive X-ray beams emitted by an X-ray tube that have traversed the ROI of the target subject efficiently at one detector position (or one source position). In some embodiments, if the detector cannot cover the entire ROI of the target subject (e.g., an area of the ROI is greater than an area of the detector), the processing device 120 may determine a center of the ROI as the target position of the detector based on the target region. Alternatively, based on the target region, the processing device 120 may determine a plurality of target positions of the detector at each of which the detector may cover a specific portion of the ROI. The processing device 120 may cause the detector to move to each of the plurality of target positions to obtain an image of the corresponding specific portion of the ROI of the target subject. The processing device 120 may further generate an image of the ROI of the target subject by combining a plurality of images corresponding to the different portions of the ROI.

In some embodiments, the target region corresponding to the ROI of the target subject may be identified from the subject model according to various approaches. For example, the processing device 120 may identify one or more feature points corresponding to the ROI of the target subject from the subject model. A feature point corresponding to the ROI may include a pixel or voxel in the subject model corresponding to a representative physical point of the ROI. Different ROIs of the target subject may have their corresponding representative physical or anatomical point(s). Merely by way of example, one or more representative physical points corresponding to the chest of the target subject may include the ninth thoracic vertebra (i.e., the spine T9), the eleventh thoracic vertebra (i.e., the spine T11), and the third lumbar vertebra (i.e., the spine L3). One or more representative physical points corresponding to the right leg of the target subject may include the right knee. Taking the chest of the target subject as an exemplary ROI, as shown in FIG. 11A, a feature point 3 corresponding to the spine T9, a feature point 4 corresponding to the spine T11, and a feature point 5 corresponding to the spine L3 may be identified from the subject model. The processing device 120 may further determine the target region of the subject model based on the one or more identified feature points. For example, the processing device 120 may determine a region in the subject model that encloses the one or more identified feature points as the target region. More descriptions of the determination of the target region based on the one or more identified feature points may be found elsewhere in the present disclosure (e.g., FIG. 11A and descriptions thereof).

As another example, the processing device 120 may divide the subject model into a plurality of regions (e.g., a region 1, a region 2, . . . , and a region 10 as illustrated in FIG. 11B). The processing device 120 may select the target region corresponding to the ROI of the target subject from the plurality of regions. More descriptions of the determination of the target region based on the plurality of regions may be found elsewhere in the present disclosure (e.g., FIG. 11B and descriptions thereof).

In some embodiments, the processing device 120 may further determine a target position of an X-ray tube based on the target position of the detector and an imaging protocol of the target subject. The X-ray tube may generate and/or emit radiation beams (e.g., X-ray beams) toward the target subject. For example, the processing device 120 may determine the target position of the X-ray tube based on the target position of the detector and a source image distance (SID) defined in the imaging protocol. The target position of the X-ray tube may include coordinates (e.g., an X-axis coordinate, a Y-axis coordinate, and/or a Z-axis coordinate) of the X-ray tube in the coordinate system 470 as shown in FIG. 4A, and/or an angle of the X-ray tube (e.g., an inclination angle of an anode target of the X-ray tube). As used herein, an SID refers to a distance between a focal spot target of the X-ray tube to an image receptor (e.g., an X-ray detector) along a beam axis of a radiation beam generated by and emitted from the X-ray tube. In some embodiments, the SID may be set manually by a user (e.g., a doctor) of the imaging system 100, or determined by one or more components (e.g., the processing device 120) of the imaging system 100 according to different situations. For example, the user may manually input information regarding the SID (e.g., a value of the SID) via a terminal device. The medical imaging device (e.g., the medical imaging device 110) may receive the information regarding the SID and set the value of the SID based on the inputted information by the user. As another example, the user may manually set the SID by controlling the movement of one or more components of the medical imaging device (e.g., the radiation source and/or the detector).

Additionally or alternatively, the processing device 120 may determine a target position of a collimator based on the target position of the X-ray tube and one or more parameters relating to a light field (e.g., a target size of the light field). More descriptions of the determination of the one or more parameters relating to the light field and the determination of the target position of the collimator may be found elsewhere in the present disclosure (e.g., FIG. 12 and descriptions thereof).

It should be noted that the above description of the determination of the target position of a movable component based on the image data is merely provided for the purposes of illustration, and not intended to limit the scope of the present disclosure. For example, the height of the target subject may be determined based on the subject model instead of the original image data, and the target position of the scanning table may be further determined based on the height of the target subject. As another example, the target position of the detector may be determined based on the original image data without generating the subject model. Merely by way of example, feature points corresponding to the ROI of the target subject may be identified from the original image data, and the target position of the detector may be determined based on the feature points identified from the original image data.

In 1030, for each of the one or more movable components of the medical imaging device, the processing device 120 (e.g., the control module 730) may cause the movable component to move to the target position of the movable component.

In some embodiments, the processing device 120 may send an instruction to the movable component, or a driving apparatus that drives the movable component to move, to cause a movable component to move to its target position. The instruction may include various parameters related to the movement of the movable component. Exemplary parameters related to the movement of the movable component may include a distance of movement, a direction of movement, a speed of movement, or the like, or any combination thereof.

Compared to a conventional way that a user needs to manually determine and/or check the position of the movable component(s) of the medical imaging device, the automated systems and methods for determining target position(s) of the movable component(s) disclosed herein may be more accurate and efficient by, e.g., reducing the workload of a user, cross-user variations, and the time needed for the system setting.

In 1040, the processing device 120 (e.g., the control module 730) may cause the medical imaging device to scan the target subject when the each of the one or more movable components of the medical imaging device is at its respective target position.

In some embodiments, before operation 1040, the target position(s) of the movable component(s) determined in operation 1030 may be further checked and/or adjusted. For example, the target position of a movable component may be manually checked and/or adjusted by a user of the imaging system 100. As yet another example, after the target subject is positioned at a scan position, target image data of the target subject may be captured using an image capturing device. The target position of a movable component (e.g., the detector) may be automatically checked and/or adjusted by one or more components (e.g., the processing device 120) of the imaging system 100 based on the target image data. For example, according to the target image data, the processing device 120 may select at least one target ionization chamber from a plurality of ionization chambers of the medical imaging device. The processing device 120 may further determine whether the target position of the detector needs to be adjusted based on the position of the at least one selected target ionization chamber. More descriptions regarding the selection of the at least one target ionization chamber may be found elsewhere in the present disclosure. See, e.g., FIGS. 16A to 16C and relevant descriptions thereof.

In some embodiments, medical image data of the target subject may be acquired during the scan of the target subject. The processing device 120 may perform one or more additional operations to process the medical image data. For example, the processing device 120 may determine an orientation of the target subject based on the medical image data, and display the medical image data according to the orientation of the target subject. More descriptions regarding the determination of the orientation of the target subject may be found elsewhere in the present disclosure. See, e.g., FIGS. 13 to 14 and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a process for preprocessing (e.g., denoising) the image data of the target subject may be added before operation 1020.

FIG. 11A is a schematic diagram illustrating an exemplary patient model 1100A of a patient according to some embodiments of the present disclosure. The patient model 1100A may be an exemplary subject model as described elsewhere in this disclosure (e.g., FIG. 9 and the relevant descriptions).

As illustrated in FIG. 11A, a plurality of feature points may be identified from the patient model. Each feature point may correspond to a physical point (e.g., an anatomical joint) of an ROI of the patient. For example, a feature point 1 may correspond to the head of the patient. A feature point 2 may correspond to the neck of the patient. A feature point 3 may correspond to the spine T9 of the patient. A feature point 4 may correspond to the spine T11 of the patient. A feature point 5 may correspond to the spine L3 of the patient. A feature point 6 may correspond to the pelvis of the patient. A feature point 7 may correspond to the right collar of the patient. A feature point 7 may correspond to the right collar of the patient. A feature point 8 may correspond to the left collar of the patient. A feature point 9 may correspond to the right shoulder of the patient. A feature point 10 may correspond to the left shoulder of the patient. A feature point 11 may correspond to the right elbow of the patient. A feature point 12 may correspond to the left elbow of the patient. A feature point 13 may correspond to the right wrist of the patient. A feature point 14 may correspond to the left wrist of the patient. A feature point 15 may correspond to the right hand of the patient. A feature point 16 may correspond to the left hand of the patient. A feature point 17 may correspond to the right hip of the patient. A feature point 18 may correspond to the left hip of the patient. A feature point 19 may correspond to the right knee of the patient. A feature point 20 may correspond to the left knee of the patient. A feature point 21 may correspond to the right ankle of the patient. A feature point 22 may correspond to the left ankle of the patient. A feature point 23 may correspond to the right foot of the patient. A feature point 24 may correspond to the left foot of the patient.

In some embodiments, a target region of the patient model 1100A corresponding to a specific ROI of the patient may be determined based on one or more feature points corresponding to the ROI. For example, the feature points 2, 3, 4, 5, and 6 may both correspond to the spine of the patient. A target region 1 corresponding to the spine of the patient may be determined by identifying the feature points 2, 3, 4, 5, and 6 from the subject model 1100A, wherein the target region 1 may enclose the feature points 2, 3, 4, 5, and 6. As another example, the feature points 3, 4, and 5 may both correspond to the chest of the patient. A target region 2 corresponding to the chest of the patient may be determined by identifying the feature points 3, 4, and 5 from the subject model 1100A, wherein the target region 2 may enclose the feature points 3, 4, and 5. As still another example, the feature point 19 may correspond to the right knee of the patient. A target region 3 corresponding to the right knee of the patient may be determined by identifying the feature point 19 from the subject model 1100A, wherein the target region 3 may enclose the feature point 19.

FIG. 11B is a schematic diagram illustrating an exemplary patient model 1100B of a patient according to some embodiments of the present disclosure.

As illustrated in FIG. 11 B, a plurality of regions (e.g., a region 1, a region 2, a region 3, a region 4, . . . , and a region 10) may be segmented from the patient model 1100B. A target region corresponding to a specific ROI may be identified in the patient model 1100B based on the plurality of regions. For example, as shown in FIG. 11B, a region covering the regions 1, 2, 3, and 4 may be identified as a target region 4 corresponding to the chest of the patient. As another example, a region covering the region 10 may be identified as a target region 5 corresponding to the right knee of the patient.

In some embodiments, an ROI of the patient may be scanned by a medical imaging device (e.g., the medical imaging device 110). A target position of a movable component (e.g., a detector) of the medical imaging device may be determined based on the target region corresponding to the ROI. More descriptions regarding the determination of the position of a movable component based on the target region may be found elsewhere in the present disclosure. See, e.g., operation 1020 and relevant descriptions thereof.

FIG. 12 is a flowchart illustrating an exemplary process for controlling a light field of a medical imaging device according to some embodiments of the present disclosure. In some embodiments, the process 1200 may be implemented in the imaging system 100 illustrated in FIG. 1. For example, the process 1200 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1200 may be accomplished with one or more additional operations not described, and/or without one or more of the operations discussed. Additionally, the order in which the operations of the process 1200 as illustrated in FIG. 12 and described below is not intended to be limiting.

In 1210, the processing device 120 (e.g., the acquisition module 710) may obtain image data of a target subject to be scanned (or examined or treated) by a medical imaging device. The image data may be captured by an imaging capture device.

Operation 1210 may be performed in a similar manner with operation 910 as described in connection with FIG. 9, and the descriptions thereof are not repeated here.

In 1220, the processing device 120 (e.g., the analyzing module 720) may determine, based on the image data, one or more parameter values of the light field.

As used herein, a light field refers to an irradiation area of radiation rays (e.g., X-ray beams) emitted from a radiation source (e.g., an X-ray source) of the medical imaging device on the target subject. The one or more parameter values of the light field may relate to one or more parameters of the light field, such as, a size, a shape, a position, or the like, or any combination thereof, of the light field. In some embodiments, a beam-limiting device (e.g., a collimator) may be positioned between the radiation source and the target subject and configured to control the one or more parameters relating to the light field. For illustration purposes, the following descriptions are described with reference to the determination of the value of the size of the light field (or referred to as a target size). This is not intended to be limiting and the systems and methods disclosed herein may be used to determine one or more other parameters relating to the light field.

In some embodiments, the processing device 120 may determine the target size of the light field based on feature information relating to an ROI of the target subject. The feature information relating to the ROI of the target subject may include a position, a height, a width, a thickness, or the like, of the ROI. As used herein, a width of an ROI refers to a length of the ROI (e.g., a length at the center of the ROI, a maximum length of the ROI) along a direction perpendicular to a sagittal plane of the target subject. A height of an ROI refers to a length of the ROI (e.g., a length at the center of the ROI, a maximum length of the ROI) along a direction perpendicular to a transverse plane of the target subject.

In some embodiments, the processing device 120 may determine feature information relating to the ROI of the target subject by identifying a target region in the image data or a subject model (or a target posture model) of the target subject generated based on the image data, wherein the target region may correspond to the ROI of the target subject. For example, the processing device 120 may generate the subject model based on the image data of the target subject, and identify the target region from the subject model. More descriptions of the identification of a target region from the image data or the subject model (or the target posture model) may be found elsewhere in the present disclosure (e.g., operation 1020 and descriptions thereof). The processing device 120 may further determine the feature information (e.g., the width and the height) of the ROI based on the target region and one or more parameters (e.g., intrinsic parameters, extrinsic parameters) of the image capturing device that captures the image data.

Additionally or alternatively, the processing device 120 may determine the feature information of the ROI of the target subject based on anatomical information associated with human. The anatomical information may include position information of one or more ROIs inside the human, size information of the one or more ROIs, shape information of the one or more ROIs, or the like, or any combination thereof. In some embodiments, the anatomical information may be acquired from a plurality of samples (e.g., images) showing the ROIs of different persons. For example, the size information of an ROI may be associated with the average size of same ROIs in the plurality of samples. Specifically, the plurality of samples may be of other persons having a similar characteristic to the patient (e.g., a similar height or weight). In some embodiments, the anatomical information associated with human may be stored in a storage device (e.g., the storage device 130, the storage device 220, the storage 390, or an external source)

After the feature information of the ROI is determined, the processing device 120 may further determine the target size of the light field based on the feature information of the ROI of the target subject. The light field with the target size may be able to cover the entire ROI of the subject model during the scan to be performed on the target subject. For example, a width of the light field may be greater than or equal to the width of the ROI, and a height of the light field may be greater than or equal to the height of the ROI.

In some embodiments, the processing device 120 may determine the target size of the light field based on a relationship between feature information of the ROI and the size of the light field (also referred to as a first relationship). Merely by way of example, the target size may be determined based on the first relationship between the height (and/or the width) of the ROI and the size of the light field. A larger height (and/or a larger width) may correspond to a larger value of the size of the light field. The first relationship between the height (and/or the width) of the ROI and the size may be represented in the form of a table or curve recording different heights (and/or widths) of the ROI and their corresponding values of the size, a mathematical function, or the like. In some embodiments, the first relationship between the height (and/or the width) of the ROI and the size may be stored in a storage device (e.g., the storage device 130, the storage device 220, the storage 390, or an external source). The processing device 120 may retrieve the first relationship from the storage device and determine the target size of the light field based on the retrieved first relationship and the height (and/or width) of the ROI.

Additionally or alternatively, the processing device 120 may determine the target size of the light field using a light field determination model. As used herein, a light field determination model refers to a model (a neural network) or algorithm configured to receive an input and output a target size of a light field of a medical imaging device based on the input. For example, the image data obtained in operation 1210 and/or the feature information of the ROI determined based on the image data may be inputted into the light field determination model, and the light field determination model may output the target size of the light field.

In some embodiments, the light field determination model may be obtained from one or more components of the imaging system 100 or an external source via a network (e.g., the network 150). For example, the light field determination model may be previously trained by a computing device (e.g., the processing device 120 ora processing device of a vendor of the light field determination model), and stored in a storage device (e.g., the storage device 130, the storage device 220, the storage 390, or an external source). The processing device 120 may access the storage device and retrieve the light field determination model. In some embodiments, the light field determination model may be trained according to a machine learning algorithm, such as an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, a rule-based machine learning algorithm, or the like, or any combination thereof. The machine learning algorithm used to generate the light field determination model may be a supervised learning algorithm, a semi-supervised learning algorithm, an unsupervised learning algorithm, or the like.

In some embodiments, the light field determination model may be trained based on a plurality of training samples. Each training sample may include sample image data of a sample subject and/or sample feature information (e.g., a height and/or width) of a sample ROI of the sample subject, and a sample size of a sample light field. As used herein, sample image data of a sample subject refers to image data of the sample subject that is used to train the light field determination model. For example, the sample image data of the sample subject may include a 2D image, point-cloud data, color image data, depth image data, or medical image data of the sample subject. The sample size of a sample light field may be used as a ground truth, which may be determined in a similar manner as how the target size of the light field is determined as described above, or manually set by a user (e.g., a doctor) based on experiences. The processing device 120 or another computing device may generate the light field determination model by training a preliminary model using the plurality of training samples. For example, the preliminary model may be trained according to a machine learning algorithm as aforementioned (e.g., a supervised machine learning algorithm).

In some embodiments, if the target size of the light field cannot cover the entire ROI of the target subject (e.g., a size of the ROI is greater than the target size of the light field), the processing device 120 may determine a plurality of light fields. Each light field may cover a specific portion of the ROI, and a total size of the plurality of light fields may be equal to or greater than the size of the ROI so that the light fields may cover the entire ROI of the target subject.

In 1230, the processing device 120 (e.g., the control module 730) may cause the medical imaging device to scan the target subject according to the one or more parameter values of the light field.

In some embodiments, the processing device 120 may determine one or more parameter values of one or more components of the medical imaging device for generating and/or controlling radiation to achieve the one or more parameter values of the light field. Merely by way of example, the processing device 120 may determine a target position of a beam-limiting device (e.g., a collimator) of the medical imaging device based on the one or more parameter values of the light field (e.g., the target size of the light field). In some embodiments, the collimator may include a plurality of leaves. The processing device 120 may determine a position of each leaf of the collimator based on the one or more parameter values of the light field. The processing device 120 may further cause the medical imaging device to adjust the component(s) for generating and/or controlling radiation according to their respective parameter value(s), and scan the target subject after the adjustment.

In some embodiments, after the one or more parameter values of the light field are determined, the processing device 120 may perform one or more additional operations to prepare for the scan on the target subject. For example, the processing device 120 may determine a value of an estimated dose associated with the target subject based at least partially on the one or more parameter values of the light field. More descriptions regarding the dose estimation may be found elsewhere in the present disclosure. See, e.g., FIG. 15 and relevant descriptions thereof. As another example, the one or more parameter values of the light field determined in process 1200 may further be checked and/or adjusted after the target subject is positioned at a scan position.

Using the automated light field control systems and methods disclosed herein, the light field may be controlled in a more accurate and efficient manner by, e.g., reducing the workload of a user, cross-user variations, and the time needed for the light field control.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a process for preprocessing (e.g., denoising) the image data of the target subject may be added before operation 1220.

FIG. 13 is a flowchart illustrating an exemplary process for determining an orientation of a target subject according to some embodiments of the present disclosure. In some embodiments, the process 1300 may be implemented in the imaging system 100 illustrated in FIG. 1. For example, the process 1300 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1300 may be accomplished with one or more additional operations not described, and/or without one or more of the operations discussed. Additionally, the order in which the operations of the process 1300 as illustrated in FIG. 13 and described below is not intended to be limiting.

In 1310, the processing device 120 (e.g., the acquisition module 710) may obtain a first image of the target subject.

As used herein, a first image of the target subject refers to an original image captured using an image capturing device (e.g., the image capturing device 160) or a medical imaging device (e.g., the medical imaging device 110). For example, the first image may be captured by a camera after the target subject is positioned at a scan position. As another example, the first image may be generated based on medical image data acquired by an X-ray imaging device in an X-ray scan of the target subject.

In some embodiments, the processing device 120 may obtain the first image from the image capturing device or the medical imaging device. Alternatively, the first image may be acquired by the image capturing device or the medical imaging device and stored in a storage device (e.g., the storage device 130, the storage device 220, the storage 390, or an external source). The processing device 120 may retrieve the first image from the storage device.

In 1320, the processing device 120 (e.g., the analyzing module 720) may determine an orientation of the target subject based on the first image.

As used herein, an orientation of the target subject refers to a direction from an upper portion (also referred to as a head portion) of the target subject to a lower portion (also referred to as a feet portion) of the target subject or from the lower portion to the upper portion. Normally, a human or a portion of human (e.g., an organ) may have an upper portion that is closer to the head of human and a lower portion that is closer to the feet of human. The upper portion and the lower portion of a human part may be defined according to the human anatomy. For example, for a hand of the target subject, a finger of the hand may correspond to the lower portion of the hand, and the wrist of the hand may correspond to the upper portion of the hand.

In some embodiments, the orientation of the target subject may include a “head up” orientation, a “head down” orientation, a “head left” orientation, and a “head right” orientation, or the like, or any combination thereof. For example, the target subject may be placed on the scanning table 410 as shown in FIG. 4A. The four edges of the scanning table 410 may be denoted as an upper edge, a lower edge, a left edge, and a right edge, respectively. For the target subject having a “head up” orientation, the upper portion of the target subject may be closer to the upper edge of the scanning table 410, and the lower portion of the target subject may be closer to the lower edge of the scanning table 410. In other words, the direction from the upper portion to the lower portion of the target subject may be (substantially) the same as the direction from the upper edge to the lower edge of the scanning table 410. For the target subject having a “head down” orientation, the upper portion of the target subject may be closer to the lower edge of the scanning table 410, and the lower portion of the target subject may be closer to the upper edge of the scanning table 410. In other words, the direction from the upper portion to the lower portion of the target subject may be (substantially) the same as the direction from the lower edge to the upper edge of the scanning table 410. For the target subject having a “head right” orientation, the upper portion of the target subject may be closer to the right edge of the scanning table 410, and the lower portion of the target subject may be closer to the left edge of the scanning table 410. In other words, the direction from the upper portion to the lower portion of the target subject may be (substantially) the same as the direction from the right edge to the left edge of the scanning table 410. For the target subject having a “head left” orientation, the upper portion of the target subject may be closer to the left edge of the scanning table 410, and the lower portion of the target subject may be closer to the right edge of the scanning table 410. In other words, the direction from the upper portion to the lower portion of the target subject may be (substantially) the same as the direction from the left edge to the right edge of the scanning table 410. It should be noted that the above descriptions regarding the orientation of the target subject is merely provided for illustration purposes, and not intended to be limiting. For example, any edge of the scanning table 410 may be regarded as the upper edge.

In some embodiments, each side of the first image may correspond to a reference object in the imaging system 100. For example, the upper side of the first image may correspond to the upper edge of the scanning table, the lower side of the first image may correspond to the lower edge of the scanning table, the left side of the first image may correspond to the left edge of the scanning table, and the right side of the first image may correspond to the right edge of the scanning table. The correspondence relationship between a side of the first image and its corresponding reference object in the imaging system 100 may be manually set by a user of the imaging system 100, or determined by one or more components (e.g., the processing device 120) of the imaging system 100.

In some embodiments, the processing device 120 may determine an orientation of a target region corresponding to an ROI of the target subject in the first image. The ROI of the target subject may be the entire target subject itself or a portion thereof. For example, the processing device 120 may identify a plurality of feature points corresponding to the ROI from the first image. A feature point corresponding to the ROI may include a pixel or voxel in the first image corresponding to a representative physical point of the ROI. Different ROIs of the target subject may have their corresponding representative physical point(s). Merely by way of example, one or more representative physical points corresponding to a hand of the target subject may include a finger (e.g., a thumb, an index finger, a middle finger, a ring finger, and a little finger) and the wrist. A finger and the wrist of a hand may correspond to the upper portion and the lower portion of the hand, respectively. The plurality of feature points may be identified manually by a user (e.g., a doctor) and/or determined by a computing device (e.g., the processing device 120) automatically according to an image analysis algorithm (e.g., an image segmentation algorithm, a feature point extraction algorithm).

The processing device 120 may then determine the orientation of the target region based on the plurality of feature points. For example, the processing device 120 may determine the orientation of the target region based on relative positions between the plurality of feature points. The processing device 120 may further determine the orientation of the target subject based on the orientation of the target region. For example, the orientation of the target region may be designated as the orientation of the target subject.

Taking the determination of an orientation of a hand in the first image as an example, the processing device 120 may identify a first feature point corresponding to a middle finger (as an exemplary lower portion of the hand) and a second feature point corresponding to the wrist of the hand (as an exemplary upper portion of the hand) from the first image. The processing device 120 may determine a direction from the first feature point to the second feature point as the orientation of a target region corresponding to the hand in the first image. The processing device 120 may further determine the orientation of the hand based on the orientation of the target region in the first image and the correspondence relationship between the sides of the first image and their respective reference objects in the imaging system 100 (also referred to as a second relationship). Merely by way of example, if the orientation of the target region corresponding to the hand (i.e., the direction from the wrist to the middle finger) is from the upper side to the lower side of the first image, the upper side of the first image corresponds to the upper edge of the scanning table, and the lower side of the first image corresponds to the lower edge of the scanning table, the processing device 120 may determine that the orientation of the hand is “head up.”

In some embodiments, the processing device 120 may determine a position of the target region corresponding to the ROI of the target subject in the first image, and determine the orientation of the target subject based on the position of the target region. For example, the target subject may be a patient and the ROI may be the head of the patient. The processing device 120 may identify a target region corresponding to the head of the target subject from the first image according to an image analysis algorithm (e.g., an image segmentation algorithm). The processing device 120 may determine a position of a center of the identified target region as the position of the target region. Based on the position of the target region, the processing device 120 may further determine which side of the first image is closest to the target region in the first image. Merely by way of example, if in the first image, the target region is closest to the upper side of the first image, and the upper side of the first image corresponds to the upper edge of the scanning table, the processing device 120 may determine that the orientation of the patient is “head up.”

In 1330, the processing device 120 (e.g., the control module 730) may cause a terminal device (e.g., the terminal device 140) to display a second image of the target subject based on the first image and the orientation of the target subject. A representation of the target subject may have a reference orientation in the second image.

As used herein, a reference orientation of the target subject refers to an expected or intended direction from an upper portion to a lower portion of the target subject or from the lower portion to the upper portion of the target subject displayed in the second image. For example, in order to make the second image compatible with an image display convention or a reading habit of a user (e.g., a doctor), the reference orientation may be a “head up” orientation. In some embodiments, the reference orientation may be manually set by a user (e.g., a doctor) or determined by one or more components (e.g., the processing device 120) of the imaging system 100. For example, the reference orientation may be determined by the processing device 120 by analyzing the image browsing history of the user.

In some embodiments, the processing device 120 may generate the second image of the target subject based on the first image and the orientation of the target subject, and transmit the second image to the terminal device for display. For example, the processing device 120 may determine a display parameter based on the first image and the orientation of the target subject. The display parameter may include a rotation angle and/or a rotation direction of the first image. For example, the target subject has a “head down” orientation and the reference orientation is the “head up” orientation, the processing device 120 may determine that the first image needs to be rotated by 180 degrees clockwise. The processing device 120 may generate the second image by rotating the first image by 180 degrees clockwise. For illustration purposes, the processing device 120 may rotate the first image by 180 degrees clockwise, and transmit a rotated first image (also referred to as the second image or an adjusted first image) to the terminal device for display.

In some embodiments, the processing device 120 may add at least one annotation indicating the orientation of the target subject on the second image, and transmit the second image with the at least one annotation to the terminal device for display. For example, an annotation “R” representing the right side of the target subject and/or an annotation “L” representing the left side of the target subject may be added to the second image.

In some embodiments, the processing device 120 may transmit the first image and the orientation of the target subject to the terminal device. The terminal device may generate the second image of the target subject based on the first image and the orientation of the target subject. For example, the terminal device may determine the display parameter based on the first image and the orientation of the target subject. The terminal device may then generate the second image based on the first image and the display parameter, and display the second image. Merely by way of example, the terminal device may adjust (e.g., rotate) the first image based on the display parameter, and display an adjusted (rotated) first image (also referred to as the second image).

In some embodiments, the processing device 120 may determine the display parameter based on the first image and the orientation of the target subject. The processing device 120 may transmit the first image and the display parameter to the terminal device. The terminal device may generate the second image of the patient based on the first image and the display parameter. The terminal device may further display the second image. Merely by way of example, the terminal device may adjust (e.g., rotate) the first image based on the display parameter, and display an adjusted (rotated) first image (also referred to as the second image).

According to some embodiments of the present disclosure, the orientation of the target subject may be determined based on the first image, and the first image may be rotated to generate the second image representing the target subject with the reference orientation if the orientation of the target subject is inconsistent with the reference orientation. In this way, the displayed second image may be convenient for the user to view. In addition, the annotation indicating the orientation of the target subject may be added on the second image, and accordingly, the user may process the second image more accurately and efficiently.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a process for preprocessing (e.g., denoising) the first image of the target subject may be added before operation 1320.

FIG. 14 is a schematic diagram illustrating exemplary images 1401, 1402, 1403, and 1404 of a hand of different orientations according to some embodiments of the present disclosure.

As illustrated in FIG. 14, in the image 1401, a direction from the wrist to the fingers of the hand is (substantially) the same as a direction from the lower side to the upper side of the image 1401. In the image 1402, a direction from the wrist to the fingers of the hand is (substantially) the same as a direction from the upper side to the lower side of the image 1402. In the image 1403, a direction from the wrist to the fingers of the hand is (substantially) the same as a direction from the right side to the left side of the image 1403. In the image 1404, a direction from the wrist to the fingers of the hand is (substantially) the same as a direction from the left side to the right side of the image 1404.

It is assumed that the upper side, the lower side, the left side, and the right side of an image (e.g., the image 1401, the image 1402, the image 1403, and the image 1404) correspond to the upper edge, the lower edge, the left edge, and the right edge of a scanning table that supports the hand, respectively. The orientations of the hand in the images 1401 to 1404 may be “head down,” “head up,” “head right,” and “head left,” respectively.

FIG. 15 is a flowchart illustrating an exemplary process for dose estimation according to some embodiments of the present disclosure. In some embodiments, the process 1500 may be implemented in the imaging system 100 illustrated in FIG. 1. For example, at least a portion of the process 1500 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1500 may be accomplished with one or more additional operations not described, and/or without one or more of the operations discussed. Additionally, the order in which the operations of the process 1500 as illustrated in FIG. 15 and described below is not intended to be limiting.

In 1510, the processing device 120 (e.g., the acquisition module 710) may obtain at least one parameter value of at least one scanning parameter relating to a scan to be performed on a target subject.

For instance, the scan may be a CT scan, an X-ray scan, or the like, to be performed by a medical imaging device (e.g., the medical imaging device 110). The at least one scanning parameter may include a voltage of a radiation source (denoted as kV) of the medical imaging device, a current of the radiation source (denoted as mA), an exposure time of the scan (denoted as ms), a size of a light field, a scan mode, a table moving speed, a gantry rotation speed, a field of view (FOV), a distance between the radiation source and a detector (also referred to as a source image distance, or an SID) or the like, or any combination thereof.

In some embodiments, the at least one parameter value may be obtained according to an imaging protocol of the target subject with respect to the scan. The imaging protocol may include information relating to the scan and/or the target subject, for example, value(s) or value range(s) of the at least one scanning parameter (or a portion thereof), a portion of the target subject to be imaged, feature information of the target subject (e.g., the gender, the body shape, the thickness), or the like, or any combination thereof. The imaging protocol may be previously generated (e.g., manually input by a user or determined by the processing device 120) and stored in a storage device. The processing device 120 may receive the imaging protocol from the storage device, and determine the at least one parameter value based on the imaging protocol.

In some embodiments, the processing device 120 may determine the at least one parameter value based on an ROI. The ROI refers to a region of the target subject to be scanned or a portion thereof. Merely by way of example, different ROIs of human may have different default scanning parameter values, and the processing device 120 may determine the at least one parameter value according to the type of the ROI to be imaged. In some embodiments, the processing device 120 may determine the at least one parameter value based on feature information of the ROI. The feature information of the ROI may include a position, a height, a width, a thickness, or the like, of the ROI. For example, the feature information of the ROI may be determined based on image data of the target subject captured by an image capturing device. More descriptions regarding the determination of the feature information of the ROI based on the image data may be found elsewhere in the present disclosure, for example, in operation 1220 and the descriptions thereof.

For illustration purposes, the determination of the values of the kV and the mA based on the thickness of the ROI is described as an example hereinafter. In some embodiments, the ROI may include different organs and/or tissue. The thickness values of different portions (e.g., different organs or tissue) in the ROI may vary. The thickness of the ROI may be, e.g., an average thickness of the different portions of the ROI.

In some embodiments, the processing device 120 may obtain a plurality of historical protocols of a plurality of historical scans performed on the same subject or one or more other subjects (each referred to as a sample subject). Each of the plurality of historical protocols may include at least one historical parameter value of the at least one scanning parameter relating to a historical scan performed on a sample subject, wherein the historical scan is of a same type of scan as the scan to be performed on the target subject. Optionally, each historical protocol may further include feature information relating to the corresponding sample subject (e.g., an ROI of the sample subject, the gender of the sample subject, the body shape of the sample subject, the thickness of the ROI of the sample subject).

In some embodiments, the processing device 120 may select one or more historical protocols from the plurality of historical protocols based on feature information associated with the target subject (e.g., the ROI of the target subject to be imaged and thickness value of the ROI) and the information relating to the sample subject of each historical protocol. Merely by way of example, the processing device 120 may select one historical protocol, the sample subject of which has the highest degree of similarity as the target subject, among the plurality of historical protocols. The degree of similarity between a sample subject and the target subject may be determined based on the feature information of the sample subject and the feature information of the target subject, for example, in a similar manner as how a degree of similarity between the target subject and a candidate subject is determined as described in connection with operation 830. For a certain scanning parameter, the processing device 120 may further designate the historical parameter value of the certain scanning parameter in the selected historical protocol as the parameter value of the scanning parameter. As another example, the processing device 120 may modify the historical parameter value of the certain scanning parameter in the selected historical protocol based on the feature information of the target subject and the sample subject, for example, a thickness difference between the ROI of the target subject and the ROI of the sample subject. The processing device 120 may further designate the modified historical parameter value of the certain scanning parameter as the parameter value of the certain scanning parameter. More descriptions regarding the determination of a parameter value of a scanning parameter based on a plurality of historical protocols may be found, for example, in Chinese Application No. 202010185201.9 entitled “Systems and methods for determining acquisition parameters of a radiation device”, filed on Mar. 17, 2020, and Chinese Application No. 202010374378.3 entitled “Systems and methods for medical image acquisition”, filed on May 6, 2020, the contents of each of which are hereby incorporated by reference.

In some embodiments, the processing device 120 may use a parameter value determination model to determine the at least one parameter value based on the ROI of the target subject and the thickness of the ROI.

In 1520, the processing device 120 (e.g., the acquisition module 710) may obtain a relationship between a reference dose and the at least one scanning parameter (also referred to as a third relationship). In some embodiments, the reference dose may indicate a dose per unit area to be delivered to the target subject. Alternatively, the reference dose may indicate a total amount of the dose to be delivered to the target subject. For instance, the third relationship may be previously generated by a computing device (e.g., the processing device 120 or another processing device) and stored in a storage device (e.g., the storage device 130 or an external storage device). The processing device 120 may obtain the third relationship from the storage device.

In some embodiments, the third relationship between the reference dose and the at least one scanning parameter may be determined by performing a plurality of reference scans on a reference subject. For instance, the processing device 120 may obtain a plurality of sets of reference values of the at least one scanning parameter. Each set of the plurality of sets of reference values may include a reference value of each of the at least one scanning parameter. For each set of the plurality of sets of reference values, a medical imaging device (e.g., the medical imaging device 110) may perform a reference scan on the reference subject according to the set of reference values, and a value of the reference dose may be measured during the reference scan. For example, the reference subject may be the air, and a radiation dosimeter may be used to measure the value of the reference dose during the reference scan. The processing device 120 (e.g., the analyzing module 720) may determine the third relationship based on the plurality of sets of reference values of the at least one scanning parameter and the plurality of values of the reference dose corresponding to the plurality of sets of reference values.

In some embodiments, the processing device 120 may determine the third relationship by performing at least one of a mapping operation, a fitting operation, a model training operation, or the like, or any combination thereof, on the sets of reference values of the at least one scanning parameter and the values of the reference dose corresponding to the sets of reference values. For example, the third relationship may be presented in the form of a table recording the plurality of sets of reference values of the at least one scanning parameter and their corresponding values of the reference dose. As another example, the third relationship may be presented in the form of a fitting curve or a fitting function that describes how the value of the reference dose changes with the reference value of the at least one scanning parameter. As yet another example, the third relationship may be presented in the form of a dose estimation model. A plurality of second training samples may be generated based on the sets of reference values of the at least one scanning parameter and their corresponding values of the reference dose. The dose estimation model may be obtained by training a second preliminary model using the second training samples according to a machine learning algorithm as described elsewhere in this disclosure (e.g., FIG. 12 and the relevant descriptions).

Merely by way of example, the at least one parameter value may include the kV, the mA, and the ms. A first set of reference values may include a first value of the kV (denoted as kV1), a first value of the mA (denoted as mA1), and a first value of the ms (denoted as ms1). A second set of reference values may include a second value of the kV (denoted as kV2), a second value of the mA (denoted as mA2), and a second value of the ms (denoted as ms2). A first reference scan may be performed by scanning the air with the first set of reference values, and the radiation dosimeter may measure a total dose or a dose per unit area in the first scan as a first value of the reference dose corresponding to the first set of reference values. A second reference scan may be performed by scanning the air with the second set of reference values, and the radiation dosimeter may measure a total dose or a dose per unit area in the second scan as a second value of the reference dose corresponding to the second set of reference values. For example, the third relationship may be presented in a table, which includes a first column recording the kV1, the mA1, the ms1, and the first value of the reference dose, and a second column recording the kV2, the mA2, the ms2, and the second value of the reference dose. As another example, the kV1, the mA1, the ms1, and the first value of the reference dose may be regarded as a training sample S1, and the kV2, the mA2, the ms2, and the second value of the reference dose may be a training sample S2. The training samples S1 and S2 may be used as second training samples in generating the dose estimation model.

In 1530, the processing device 120 (e.g., the analyzing module 720) may determine, based on the third relationship and the at least one parameter value of the at least one scanning parameter, a value of an estimated dose associated with the target subject.

In some embodiments, the reference dose may indicate the total amount of dose. The processing device 120 may determine a value of the reference dose corresponding to the at least one parameter value of the at least one scanning parameter based on the third relationship. The processing device 120 may further designate the value of the reference dose as the value of the estimated dose.

In some embodiments, the reference dose may indicate the dose per unit area. The processing device 120 may determine a value of the reference dose corresponding to the at least one parameter value of the at least one scanning parameter based on the third relationship and the at least one parameter value. For example, the processing device 120 may determine the value of the reference dose corresponding to the at least one parameter value of the at least one scanning parameter by looking up a table recording the third relationship or inputting the at least one parameter value of the at least one scanning parameter into a dose estimation model. The processing device 120 may further obtain a size (or area) of the light field relating to the scan. For example, the processing device 120 may determine the size (or area) of the light field by performing one or more operations of the process 1200 as described in connection with FIG. 12. As another example, the size (or area) of the light filed may be previously determined, e.g., manually by a user or another computing device, and stored in a storage device. The processing device 120 may obtain the size (or area) of the light field from the storage device. The processing device 120 may then determine the value of the estimated dose based on the size (or area) of the light field and the value of the dose per unit area. For example, the processing device 120 may determine a product of the size (or area) of the corresponding light field and the corresponding value of the dose per unit area as the value of the estimated dose.

In some embodiments, the estimated dose may include a first estimated dose to be delivered to the target subject during the scan, which may be determined, for example, based on the size of the light field and the value of the dose per unit area as aforementioned. In some embodiments, the processing device 120 may further determine a value of a second estimated dose based on the first estimated dose. The second estimated dose may indicate a dose to be absorbed by the target subject (or a portion thereof) during the scan.

In some embodiments, a plurality of ROIs of the target subject may be scanned. For each of the plurality of ROIs, the processing device 120 may determine a value of a second estimated dose to be absorbed by the ROI during the scan. For instance, for each of the plurality of ROIs, the processing device 120 may obtain a thickness and an attenuation coefficient of the ROI. The processing device 120 may further determine a value of a second estimated dose to be absorbed by the corresponding ROI during the scan based on the value of the first estimated dose, the thickness of the ROI, and the attenuation coefficient of the ROI. Additionally or alternatively, the processing device 120 may further generate a dose distribution map based on the values of the second estimated dose of the plurality of ROIs. The dose distribution map may illustrate the distribution of an estimated dose to be absorbed by different ROIs during the scan in a more intuitive and efficient way. For instance, in the dose distribution map, a plurality of ROIs may be displayed in different colors according to their respective values of the second estimated dose. As another example, if the value of the second estimated dose of an ROI exceeds an absorbed dose threshold, the ROI may be marked by a specific color or an annotation for reminding the user that the parameter value of the at least one scanning parameter may need to be checked and/or adjusted.

Optionally, the processing device 120 may determine a total estimated dose to be absorbed by the target subject. In some embodiments, the processing device 120 may determine the total estimated dose to be absorbed by the target subject by summing up the values of the second estimated dose of the ROI(s). Additionally or alternatively, different ROIs (e.g., different organs or tissue of the target subject) may correspond to different thickness values and/or different values of the attenuation coefficient. The processing device 120 may determine an average thickness of the plurality of ROIs and an average attenuation coefficient of the plurality of ROIs.

The first estimated dose and/or the second estimated dose(s) may be used to evaluate whether the at least one parameter value of the at least one scanning parameter obtained in operation 1510 is appropriate. For example, an inadequate first estimated dose (e.g., less than a first dose threshold) may indicate a reduced quality of an image generated based on scan data acquired in the scan. As another example, the second estimated dose of an ROI exceeding a second dose threshold may indicate that the ROI may be subject to excessive damage. By determining the first estimated dose and/or the second estimated dose(s) and subsequently evaluating the at least one scanning parameters, some problems (such as a relatively low quality of the generated image and/or excessive damage to the target subject) may be avoided. Compared to a conventional way that a user needs to manually determine the first estimated dose and/or the second estimated dose, the automated dose estimation systems and methods disclosed herein may be more accurate and efficient by, e.g., reducing the workload of a user, cross-user variations, and the time needed for the selection of the at least one target ionization chamber.

In 1540, the processing device 120 (e.g., the analyzing module 720) may determine whether the estimated dose (e.g., the first estimated dose) is greater than a dose threshold (e.g., a dose threshold with respect to the first estimated dose). In response to determining that the estimated dose is greater than the dose threshold, the processing device 120 may proceed to operation 1550 to determine that the parameter value of the at least one scanning parameter needs to be adjusted.

In response to determining that the estimated dose is less than (or equal to) the dose threshold, the processing device 120 may determine that the parameter value of the at least one scanning parameter does not need to be adjusted. Optionally, the processing device 120 may perform 1560 to send a control signal to the medical imaging device to cause the medical imaging device to perform the scan on the target subject based on the at least one parameter value of the at least one scanning parameter. In some embodiments, the dose threshold may be a preset value stored in a storage device (e.g., the storage device 130) or set by a user manually. Alternatively, the dose threshold may be determined by the processing device 120. Merely by way of example, the dose threshold may be selected from a plurality of candidate dose thresholds based on the gender, the age, and/or other reference information of the target subject.

In some embodiments, the processing device 120 may transmit the dose evaluation result (e.g., value of the first estimated dose, the value of the second estimated dose(s), and/or the dose distribution map) to a terminal device (e.g., the terminal device 140). A user may view the dose evaluation result via the terminal device. Optionally, the user may further input a response regarding whether the parameter value of the at least one scanning parameter needs to be adjusted.

In 1550, in response to determining that the estimated dose exceeds the dose threshold, the processing device 120 (e.g., the analyzing module 720) may determine that the parameter value of the at least one scanning parameter needs to be adjusted.

In some embodiments, the processing device 120 may send a notification to the terminal device to notify the user that the parameter value of the at least one scanning parameter needs to be adjusted. The user may manually adjust the parameter value of the at least one scanning parameter. Merely by way of example, the user may adjust (e.g., reduce or increase) the parameter value of the voltage of the radiation source, the parameter value of the current of the radiation source, the parameter value of the exposure time, the SID, or the like, or any combination thereof.

In some embodiments, the processing device 120 may send a control signal to cause the medical imaging device to adjust the parameter value of the at least one scanning parameter. For instance, the control signal may cause the medical imaging device to reduce the parameter value of the current of the radiation source by, for example, 10 milliamperes.

In 1560, in response to determining that the estimated dose does not exceed the dose threshold, the processing device 120 (e.g., the control module 730) may cause the medical imaging device (e.g., the medical imaging device 110) to perform the scan on the target subject based at least in part on the at least one parameter value of the at least one scanning parameter. For example, the processing device 120 may transmit the at least one parameter value obtained in operation 1510 and/or parameter values of other parameters associated with the scan (e.g., the target position of the scanning table or the target position of the detector determined in operation 1030 of FIG. 10) to the medical imaging device. In some embodiments, the process 1500 (or a portion thereof) may be performed before, during, or after the target subject is placed at the scan position for receiving the scan.

In some embodiments, after the at least one parameter value of the at least one scanning parameter is adjusted, the processing device 120 may generate updated parameter value(s) of the at least one scanning parameter. The processing device 120 may transmit the updated parameter value(s) to the medical imaging device. The medical imaging device may perform the scan based on at least in part on the updated parameter value(s).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, operations 1540-1560 may be omitted. In some embodiments, operations in the process 1500 may be performed in a different order. For instance, operation 1520 may be performed before operation 1510.

FIG. 16A is a flowchart illustrating an exemplary process for selecting a target ionization chamber among a plurality of ionization chambers according to some embodiments of the present disclosure. In some embodiments, the process 1600A may be implemented in the imaging system 100 illustrated in FIG. 1. For example, the process 1600A may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1600A may be accomplished with one or more additional operations not described, and/or without one or more of the operations discussed. Additionally, the order in which the operations of the process 1600A as illustrated in FIG. 16A and described below is not intended to be limiting.

In 1610, the processing device 120 (e.g., the acquisition module 710) may obtain target image data of a target subject to be scanned by a medical imaging device. The medical imaging device may include a plurality of ionization chambers. In some embodiments, the medical imaging device (e.g., the medical imaging device 110) may be a suspended X-ray medical imaging device, a digital radiography (DR) device (e.g., a mobile digital X-ray medical imaging device), a C-arm device, a CT device, or the like, as described elsewhere in the present disclosure.

In some embodiments, the target image data may be captured by an imaging capturing device (e.g., the imaging capturing device 160) after a target subject is positioned at a scan position for receiving a scan by the medical imaging device. For example, the process 1600A may be performed after one or more movable components (e.g., a detector) of the medical imaging device are moved to their respective target positions. The target position(s) of the one or more movable components may be determined, for example, in a similar manner as operations 1010-1030. As another example, the process 1600A may be performed before or after the process 1500 for dose estimation.

The target image data may include 2D image data, 3D image data, depth image data, or the like, or any combination thereof. In some embodiments, the processing device 120 may transmit an instruction to the image capturing device to capture image data of the target subject after the target subject is positioned at the scan position. In response to the instruction, the image capturing device may capture image data of the target subject as the target image data and transmit the captured target image data to the processing device 120 directly or via a network (e.g., the network 150). As another example, the image capturing device may be directed to capture image data of the target subject continuously or intermittently (e.g., periodically) after the target subject is positioned at the scan position. In some embodiments, after the image capturing device captures image data, the image capturing device may transmit the image data to the processing device 120 as the target image data for further analysis. In some embodiments, the acquisition of the target image data by the image capturing device, the transmission of the captured target image data to the processing device 120, and the analysis of the target image data may be performed substantially in real-time so that the target image data may provide information indicating a substantially real-time status of the target subject.

An ionization chamber of the medical imaging device may be configured to detect an amount of radiation (e.g., an amount of radiation per unit area per unit time) that reaches the detector of the medical imaging device. For example, the plurality of ionization chambers may include a vented chamber, a sealed low pressure chamber, a high pressure chamber, or the like, or any combination thereof. In some embodiments, at least one target ionization chamber may be selected among the plurality of ionization chambers (as will be described in connection with operation 1620). The at least one target ionization chamber may be actuated during the scan of the target subject, while other ionization chamber(s) (if any) may be shut down during the scan of the target subject.

In 1620, the processing device 120 (e.g., the analyzing module 720) may select, among the plurality of ionization chambers, the at least one target ionization chamber based on the target image data.

In some embodiments, the processing device 120 may select a single target ionization chamber among the plurality of ionization chambers. Alternatively, the processing device 120 may select multiple target ionization chambers among the plurality of ionization chambers. For example, the processing device 120 may compare a size (e.g., an area) of a light filed relating to the scan with a size threshold. In response to determining that the size of the light field is greater than the size threshold, the processing device 120 may select two or more target ionization chambers among the plurality of ionization chambers. As another example, if there are at least two organs of interest in the ROI, the processing device 120 may select at least two target ionization chambers among the plurality of ionization chambers. An organ at interest refers to a specific organ or tissue of the target subject. Merely by way of example, if the ROI includes the chest, the processing device 120 may select two target ionization chambers from the plurality of ionization chambers, wherein one of the target ionization chambers may correspond to the left lung of the target subject and the other one of the target ionization chambers may correspond to the right lung of the target subject.

In some embodiments, the processing device 120 may select at least one candidate ionization chamber corresponding to the ROI among the plurality of ionization chambers based on the target image data and position information of the plurality of ionization chambers. The processing device 120 may further select the target ionization chamber(s) from the candidate ionization chamber(s). Merely by way of example, the processing device 120 (e.g., the analyzing module 720) may generate a target image (e.g., a first target image as described in connection with FIG. 16B and/or a second target image as described in connection with FIG. 16C) based at least in part on the target image data and select the target ionization chamber(s) from the candidate ionization chamber(s) based on the target image.

In some embodiments, the processing device 120 may select the target ionization chamber by performing one or more operations of process 1600B as described in connection with FIG. 16B and/or process 1600C as described in connection with FIG. 16C.

In 1630, the processing device 120 (e.g., the control module 730) may cause the medical imaging device to scan the target subject using the at least one target ionization chamber.

For example, the processing device 120 may transmit an instruction to the medical imaging device to direct the medical imaging device to start the scan. The instruction may include information regarding the at least one target ionization chamber, such as an identification number of each of the at least one target ionization chamber, the position of each of the at least one target ionization chamber, or the like. Optionally, the instruction may further include parameter value(s) for one or more parameters relating to the scan. For instance, the one or more parameters may include the current of the radiation source, the voltage of the radiation source, the exposure time, or the like, or any combination thereof. In some embodiments, the current of the radiation source, the voltage of the radiation source, and the exposure time may be determined by the processing device 120 by performing one or more operations of the process 1500 as described in connection with FIG. 15.

In some embodiments, an automatic exposure control (AEC) method may be implemented during the scan of the target subject. When an accumulative mount of radiations detected by the at least one target ionization chamber exceeds a threshold amount, a radiation controller (e.g., a component of the medical imaging device or a processing device) may cause the radiation source of the medical imaging device to stop the scan.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a user (e.g., an operator) may view the target image and select the at least one target ionization chamber from the plurality of ionization chambers. The process 1600A may further include an operation in which the processing device 120 receives a user input regarding the selection of the at least one target ionization chamber.

FIG. 16B is a flowchart illustrating an exemplary process for selecting at least one target ionization chamber for an ROI of a target subject based on target image data of the target subject according to some embodiments of the present disclosure. In some embodiments, one or more operations of process 1600B may be performed to achieve at least part of operation 1620 as described in connection with FIG. 16A.

In 1640, the processing device 120 (e.g., the analyzing module 720) may select, among the plurality of ionization chambers, at least one first candidate ionization chamber that is in a vicinity of the ROI of the target subject.

In some embodiments, the processing device 120 may select one or more first candidate ionization chambers in the vicinity of the ROI from the ionization chambers as based on the distances between the ionization chambers and the ROI. The distance between an ionization chamber and the ROI refers to a distance between a point (e.g., a central point) of the ionization chamber and a point (e.g., a central point) of the ROI. The distance between an ionization chamber and the ROI may be determined based on position information of the ionization chamber and position information of the ROI. For example, the position information of the ionization chamber may include a position of the ionization chamber relative to a reference component of the medical imaging device (e.g., the detector) and/or a position of the ionization chamber in a 3D coordinate system. The position information of the ionization chamber may be stored in a storage device (e.g., the storage device 130) or determined based on the target image data. The position information of the ROI may include a position of the ROI relative to a reference component of the medical imaging device (e.g., the detector). The position information of the ROI may be determined based on the target image data, e.g., by identifying a target region in the target image data. The target region may correspond to the ROI of the target subject.

Merely by way of example, for an ionization chamber, the processing device 120 may determine a distance between the ionization chamber and the ROI. The processing device 120 may determine whether the distance is less than a distance threshold. In response to determining that the distance corresponding to the ionization chamber is less than the distance threshold, the processing device 120 may determine that the ionization chamber is in the vicinity of the ROI and designate the ionization chamber as one of the first candidate ionization chamber(s). As another example, the processing device 120 may select an ionization chamber that is closest to the ROI among the ionization chambers. The selected ionization chamber may be regarded as being located in the vicinity of the ROI and designated as one of the first candidate ionization chambers.

In 1650, for each of the first candidate ionization chamber(s), the processing device 120 (e.g., the analyzing module 720) may determine whether a position offset between the ROI and the first candidate ionization chamber is negligible based on the target image data and the position information of the first candidate ionization chamber.

As used herein, if the position offset between the ROI and a first candidate ionization chamber is negligible, the positions of the first candidate ionization chamber and the position of the ROI may be regarded as being matched, and the first candidate ionization chamber may be selected as one of the at least one target ionization chamber.

In some embodiments, for a first candidate ionization chamber, the processing device 120 may determine whether the position offset between the first candidate ionization chamber and the ROI is negligible by generating a first target image. The first target image may be indicative of the position of the first candidate ionization chamber relative to the ROI, which may be generated based on the target image data and position information of the first candidate ionization chamber(s). For example, the first target image may be generated by annotating the ROI and the first candidate ionization chamber (and optionally other first candidate ionization chamber(s)) on the target image data. As another example, a target subject model representing the target subject may be generated based on the target image data. The first target image may be generated by annotating the ROI and the at least one first candidate ionization chamber (and optionally other ionization chamber(s) from the plurality of ionization chambers) on the target subject model. Merely by way of example, the first target image may be a similar image to an image 2000 as shown in FIG. 20, in which a plurality of representations 2030 of a plurality of ionization chambers are annotated on a representation 2010 (i.e., a target subject model) of a target subject.

The processing device 120 may further determine whether a representation of the first candidate ionization chamber in the first target image is covered by a target region corresponding to the ROI in the first target image. As used herein, in an image, if a target region corresponding to the ROI covers the entire or more than a certain percentage (e.g., 99%, 95%, 90%, 80%) of a representation of the first candidate ionization chamber, the representation of the first candidate ionization chamber may be regarded as being covered by the target region. In response to determining that the representation of the first candidate ionization chamber in the first target image is covered by the target region, the processing device 120 may determine that the position offset between the first candidate ionization chamber and the ROI is negligible. In response to determining that the representation of the first candidate ionization chamber in the first target image is not covered by the target region, the processing device 120 may determine that the position offset between the first candidate ionization chamber and the ROI is not negligible (or a position offset exists between the first candidate ionization chamber and the ROI).

Additionally or alternatively, the processing device 120 may transmit the first target image to a terminal device (e.g., the terminal device 140) for displaying the first target image to a user (e.g., an operator). The user may view the first target image and provide a user input via the terminal device 140. The processing device 120 may determine whether the position offset between a first candidate ionization chamber and the ROI is negligible based on the user input. For example, the user input may indicate whether the position offset between the first candidate ionization chamber and the ROI is negligible. As another example, the user input may indicate whether the first candidate ionization chamber should be selected as a target ionization chamber.

In 1660, for each of the at least one first candidate ionization chamber, the processing device 120 (e.g., the analyzing module 720) may determine whether the first candidate ionization chamber is one of the at least one target ionization chamber based on a determination result of whether the position offset is negligible.

For a first candidate ionization chamber, in response to determining that the corresponding position offset is negligible, the processing device 120 may designate the first candidate ionization chamber as one of the target ionization chamber(s) corresponding to the ROI. In some embodiments, the processing device 120 may select the target ionization chamber(s) and annotate the selected target ionization chamber(s) in the first target image. The processing device 120 may further transmit the first target image with the annotation of the selected target ionization chamber(s) to a terminal device of a user. The user may verify the selection result of the target ionization chamber(s).

For a first candidate ionization chamber, in response to determining that the position offset is not negligible (i.e., the position offset exists), the first candidate ionization chamber may not be determined as one of the target ionization chamber(s) by the processing device 120. In some embodiments, if the position offset (i.e., a non-negligible position offset) exists for each of the first candidate ionization chamber(s), the processing device 120 may determine that a position of the ROI relative to the plurality of ionization chambers needs to be adjusted. For instance, the processing device 120 and/or the user may cause a scanning table (e.g., the scanning table 114) and/or a detector (e.g., the detector 112, the flat panel detector 440) of the medical imaging device to move so as to adjust the position of the ROI relative to the plurality of ionization chambers. As another example, the processing device 120 may instruct the target subject to move one or more body parts to adjust the position of the ROI relative to the plurality of ionization chambers. More details regarding the adjustment of the position of the ROI relative to the plurality of ionization chambers may be found elsewhere in the present disclosure, for example, in FIG. 17 and the description thereof.

In some embodiments, after the position of the ROI relative to the ionization chamber is adjusted, the processing device 120 may further select the at least one target ionization chamber among the plurality of ionization chambers based on the adjusted position of the ROI. For example, the processing device 120 may perform operation 1610 again to obtain updated target image data of the target subject after the position of the target subject is adjusted. The processing device 120 may further perform 1620 based on the updated target image data to determine the at least one target ionization chamber.

FIG. 16C is a flowchart illustrating an exemplary process for selecting at least one target ionization chamber for an ROI of a target subject based on target image data of the target subject according to some embodiments of the present disclosure. In some embodiments, one or more operations of process 1600C may be performed to achieve at least part of operation 1620 as described in connection with FIG. 16A.

In 1670, the processing device 120 (e.g., the analyzing module 720) may generate a second target image indicating positions of at least some of the plurality of ionization chambers relative to the ROI of the target subject.

For example, the at least some of the ionization chambers may include all of the plurality of ionization chambers. As another example, the plurality of ionization chambers may include a portion of the ionization chambers, which may be selected from the ionization chamber randomly or according to a specific rule. Merely by way of example, multiple sets of ionization chambers may be located in different regions (e.g., relative to the detector), such as a set of ionization chambers located in a central region, a set of ionization chambers located in a left region, a set of ionization chambers located in a right region, a set of ionization chambers located in an upper region, a set of ionization chambers located in a lower region, etc. The processing device 120 may select one or more sets from the sets of ionization chambers as the at least some of the ionization chamber. For example, if the ROI includes two organs of interest that are substantially located on both sides of the body of the target subject, such as the right lung and the left lung, the processing device 120 may select a set of at least one ionization chamber located in the left region and a set of at least one ionization chamber located in the right region as the at least some of the plurality of ionization chambers.

In some embodiments, the processing device 120 may generate the second target image by annotating the ROI and each of the at least some of the plurality of ionization chambers on the target image data. As shown in FIG. 20, one or more candidate ionization chambers 2030 may be annotated in a display image and the display image may be presented to the user via a terminal device. As another example, a subject model representing the target subject may be generated based on the target image data. The second target image may be generated by annotating the ROI and each of the at least some of the plurality of ionization chambers on the subject model. In some embodiments, the second target image may be generated by superimposing a representation of each of the at least some of the plurality of ionization chambers on a representation of the target subject (e.g., a representation of the subject model) in one image.

In 1680, the processing device 120 (e.g., the analyzing module 720) may identify at least one second candidate ionization chamber among the plurality of ionization chambers based on the second target image.

A second candidate ionization chamber refers to an ionization chamber, a representation of which in the second target image is covered by a target region corresponding to the ROI in the second target image.

In 1690, the processing device 120 (e.g., the analyzing module 720) may select the at least one target ionization chamber among the plurality of ionization chamber(s) based on an identification result of the at least one second candidate ionization chamber.

In some embodiments, the processing device 120 may determine whether there is at least one identified second candidate ionization chamber in the second target image. In response to determining that there is at least one identified second candidate ionization chamber in the second target image, the processing device 120 may select the target ionization chamber(s) corresponding to the ROI from the at least one identified second candidate ionization chamber. For example, the processing device 120 may randomly select one or more of the at least one identified second candidate ionization chamber as the target ionization chamber(s). As another example, the processing device 120 may designate one of the at least one identified second candidate ionization chamber whose central point is closest to a specific point of the ROI (e.g., a central point of the ROI or a specific tissue of the ROI) as the target ionization chamber corresponding to the ROI. As yet another example, the ROI may include the left lung and the right lung. The processing device 120 may designate one of the at least one identified second candidate ionization chamber whose central point is closest to the central point of the left lung as a target ionization chamber corresponding to the left lung. The processing device 120 may also designate one of the identified second candidate ionization chambers whose central point is closest to the central point of the right lung as a target ionization chamber corresponding to the right lung. In this way, the processing device 120 may select the target ionization chamber(s) from the plurality of ionization chambers in an automatic manner where little or no user input is needed for selecting the target ionization chamber(s). The automatic selection of the target ionization chamber(s) may reduce the workload of the user and be more accurate (e.g., insusceptible to human error or subjectivity).

In some embodiments, the processing device 120 may transmit the second target image to a terminal device of a user. The user may view the second target image via the terminal device. The processing device 120 may determine the at least one target ionization chamber corresponding to the ROI based on a user input of the user received via the terminal device. For example, the user input may indicate the target ionization chamber(s) selected from the at least one identified second candidate ionization chamber. In some embodiments, the processing device 120 may select the target ionization chamber(s) and annotate the selected target ionization chamber(s) in the second target image. The processing device 120 may further transmit the second target image with the annotation of the selected target ionization chamber(s) to the terminal device. The user may verify the selection result of the target ionization chamber(s).

In some embodiments, in response to determining that there is no identified second candidate ionization chamber in the second target image, the processing device 120 may determine that the position of the ROI relative to the plurality of ionization chambers needs to be adjusted. More details regarding the adjustment of the position of the ROI relative to the plurality of ionization chambers may be found elsewhere in the present disclosure, for example, in the description relating to operation 1660 in FIG. 16 and/or operation 1730 in FIG. 17.

According to some embodiments of the present disclosure, the systems and methods disclosed herein may generate a target image (the first target image and/or the second target image as aforementioned) that indicates a position of one or more ionization chambers (e.g., the candidate ionization chamber(s) and/or the target ionization chamber(s)) to the ROI of the target subject. Optionally, the systems and methods may further transmit the target image to a terminal device of a user to assist or check the selection of the target ionization chamber(s).

Normally, the ionization chambers for existing medical imaging devices are located between the target subject and the detector of the medical imaging device. It may be difficult for the user to directly observe positions of the ionization chambers relative to the ROI since the positions of the ionization chambers are shielded by the target subject and/or the detector (e.g., the flat panel detector 440). Through generating the target image (e.g., the first target image or the second target image), the positions of the ionization chambers (or a portion thereof) relative to the ROI may be presented in the target image. The visualization of the one or more of the plurality of ionization chambers may facilitate the selection of the target ionization chamber(s) from the ionization chambers and/or the verification of the selection result, and also improve the accuracy of the selection of the target ionization chamber(s). Compared to a conventional way that a user needs to manually select the at least one target ionization chamber from the plurality of ionization chambers, the automated target ionization chamber selection systems and methods disclosed herein may be more accurate and efficient by, e.g., reducing the workload of a user, cross-user variations, and the time needed for the selection of the at least one target ionization chamber.

FIG. 17 is a flowchart illustrating an exemplary process for subject positioning according to some embodiments of the present disclosure. In some embodiments, the process 1700 may be implemented in the imaging system 100 illustrated in FIG. 1. For example, the process 1700 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1700 may be accomplished with one or more additional operations not described, and/or without one or more of the operations discussed. Additionally, the order in which the operations of the process 1700 as illustrated in FIG. 17 and described below is not intended to be limiting.

In 1710, the processing device 120 (e.g., the acquisition module 710) may obtain target image data of a target subject holding a posture to be examined (treated or scanned). The target image data may be captured by an image capturing device.

The posture may reflect one or more of a position, a pose, a shape, a size, etc., of the target subject (or a portion thereof). In some embodiments, operation 1720 may be performed in a similar manner as operation 1610 as described in connection with FIG. 16A, and the description thereof are not repeated here.

In 1720, the processing device 120 (e.g., the acquisition module 710) may obtain a target posture model representing a target posture of the target subject. The target posture of the target subject may be also referred to as a reference posture of the target subject as described in connection with FIG. 9. The target posture of the target subject may be a standard posture that the target subject needs to hold during the scan to be performed on the target subject. The target posture model may be a 2D skeleton model, a 3D skeleton model, a 3D mesh model, or the like.

In some embodiments, the target posture model may be generated by the processing device 120 or another computing device based on a reference posture model and image data of the target subject. The image data of the target subject may be acquired prior to the capture of the target image data. For example, the image data of the target subject may be acquired before or after the target subject enters the examination room. More descriptions regarding the generation of the target posture model may be found elsewhere in the present disclosure, for example, the process 900 and descriptions thereof.

In 1730, the processing device 120 (e.g., the analyzing module 720) may determine whether the posture of the target subject needs to be adjusted based on the target image data and the target posture model.

In some embodiments, the processing device 120 may generate a target subject model based on the target image data. The target subject model may represent the target subject holding the posture. For example, the target subject model may be a 2D skeleton model, a 3D skeleton model, a 3D mesh model, or the like. In some embodiments, the model types of the target subject model and the target posture model may be the same. For example, the target subject model and the target posture model may both be 3D skeleton models. In some embodiments, the model types of the target subject model and the target posture model may be different. For example, the target subject model may be a 2D skeleton model, and the target posture model may be a 3D skeleton model. The processing device 120 may need to transform the 3D skeleton model into a second 2D skeleton model by, for example, projecting the 3D skeleton model. The processing device 120 may further compare the 2D skeleton model corresponding to the target subject model and the second 2D skeleton model corresponding to the target posture model.

The processing device 120 may then determine a matching degree between the target subject model and the target posture model. The processing device 120 may further determine whether the posture of the target subject needs to be adjusted based on the matching degree. For example, the processing device 120 may compare the matching degree with a threshold degree. The threshold degree may be, for example, 70%, 75%, 80%, 85%, etc. In response to determining that the matching degree is greater than (or equal to) the threshold degree, the processing device 120 may determine that the posture of the target subject does not need to be adjusted. In response to determining that the matching degree is below the threshold degree, the processing device 120 may determine that the posture of the target subject needs to be adjusted. Merely by way of example, the processing device 120 may further cause a notification to be generated. The notification may be configured to notify a user (e.g., an operator) that the posture of the target subject needs to be adjusted. The notification may be provided to the user via a terminal device, for example, in the form of text, voice, an image, a video, a haptic alert, or the like, or any combination thereof.

The matching degree between the target subject model and the target posture model may be determined in various approaches. Merely by way of example, the processing device 120 may identify one or more first feature points from the target subject model and identify one or more second feature points from the target posture model. The processing device 120 may further determine the matching degree between the target subject model and the target posture model based on the one or more first feature points and the one or more second feature points. For instance, the one or more first feature points may include a plurality of first pixels corresponding to a plurality of joints of the target subject. The one or more second feature points may include a plurality of second pixels corresponding to the plurality of joints of the target subject. The matching degree may be determined by comparing a first coordinate of each first pixel in the target subject model with a second coordinate of a corresponding second pixel of the first pixel in the target posture model. A first pixel and a second pixel may be regarded as corresponding to each other if they correspond to a same physical point of the target subject.

For instance, the processing device 120 may determine a distance between a first pixel and a second pixel corresponding to the first pixel based on a first coordinate of the first pixel and a second coordinate of the second pixel. The processing device 120 may compare the distance with a threshold. In response to determining that the distance is less than or equal to the threshold, the processing device 120 may determine that the first pixel is matched with the second pixel. For example, the threshold may be 0.5 cm, 0.2 cm, 0.1 cm, or the like. In some embodiments, the threshold may have a default value or a value manually set by a user. Additionally or alternatively, the threshold may be adjusted according to an actual need. In some embodiments, the processing device 120 may further determine the matching degree between the target subject model and the target posture model based on a proportion of the first pixels in the target subject model that are matched with corresponding second pixels in the target posture model. For example, if each of 70% of the first pixels in the target subject model is matched with a corresponding second pixel, the processing device 120 may determine that the matching degree between the target subject model and the target posture model is 70%.

In some embodiments, the processing device 120 (e.g., the analyzing module 720) may generate a composite image (e.g., a composite image 1800 as shown in FIG. 18) based on the target posture model and the target image data. The processing device 120 may further determine whether the posture of the target subject needs to be adjusted based on the composite image. The composite image may illustrate both the target posture model and the target subject. Merely by way of example, in the composite image, a representation of the target posture model may be superimposed on a representation of the target subject. For example, the target image data may include an image, such as a color image, an infrared image, of the target subject. The composite image may be generated by superimposing the representation of the target posture model on the representation of the target subject in the image of the target subject. As another example, a target subject model representing the target subject may be generated based on the target image data of the target subject. The composite image may be generated by superimposing the representation of the target posture model on the representation of the target subject model.

In some embodiments, the processing device 120 may determine the matching degree between the target subject model and the target posture model based on the composite image, and determine whether the posture of the target subject needs to be adjusted based on the matching degree. For example, the processing device 120 may determine, in the composite image, a proportion of the representation of the target posture model that is overlapped with the representation of the target subject model. The higher the proportion is, the higher the matching degree between the target subject model and the target posture model. The processing device 120 may further determine whether the posture of the target subject needs to be adjusted based on the matching degree and a threshold degree as aforementioned.

Additionally or alternatively, the processing device 120 may transmit the composite image to a terminal device. In some embodiments, the terminal device may include a first terminal device (e.g., a console) of a user (e.g., a doctor, an operator of the medical imaging device). The processing device 120 may receive a user input by the user regarding whether the posture of the target subject needs to be adjusted. For example, the first terminal device of the user may display the composite image to the user. The user may determine whether the posture of the target subject needs to be adjusted based on the composite image, and input his/her determination result via the first terminal device. Compared to a conventional way of determining whether the posture of the target subject needs to be adjusted by directly observing the posture of the target subject, the composite image may make it more convenient for the user to compare the posture of the target subject and a target posture (i.e., a standard posture) with respect to the scan.

Additionally or alternatively, the terminal device may include a second terminal device of the target subject (e.g., a patient). For example, the second terminal device may include a display device in the vicinity of the target subject, e.g., mounted on the medical imaging device or the ceiling of the examination room. The processing device 120 may transmit the composite image to the second terminal device. The target subject may view the composite image via the second terminal device and get information regarding the present posture he/she holds and the target posture that he/she needs to hold. In some embodiments, in response to determining that the posture of the target subject needs to be adjusted, the processing device 120 may cause an instruction to be generated. The instruction may guide the target subject to move one or more body parts of the target subject to hold the target posture. The instruction may be in the form of text, voice, an image, a video, a haptic alert, or the like, or any combination thereof. The instruction may be provided to the target subject via the second terminal device. For example, the instruction may be provided to the target subject in the form of a voice instruction, such as “please move to your left,” “please put your arms on the armrests of the medical imaging device,” etc. Additionally or alternatively, the instruction may include image data (e.g., an image, an animation) that guides the target subject to move the one or more body parts. Merely by way of example, the composite image illustrating the target posture model and the target subject may be displayed to the target subject via the second terminal device. An annotation may be provided on the composite image to indicate the one or more body parts need to be moved and/or recommended moving directions of the one or more body parts. In some embodiments, the user (e.g., an operator) may view the composite image via the first terminal device and guide the target subject to move the one or more body parts.

In some embodiments, in response to determining that the posture (e.g., the position) of the target subject needs to be adjusted, the processing device 120 may cause the position of one or more movable components to be adjusted. For instance, the one or more movable components may include a scanning table (e.g., the scanning table 114), a detector (e.g., the detector 112, the flat panel detector 440), a radiation source (e.g., a tube, the radiation source 115, the X-ray source 420), or the like, or any combination thereof. The adjustment of the position of the one or more movable components may result in a change of the position of the ROI with respect to the medical imaging device, thus modifying the posture of the target subject.

According to some embodiments of the present disclosure, a target posture model of the target subject may be generated and subsequently used in checking and/or guiding the positioning of the target subject. The target posture model may be a customizable model that has same contour parameter(s) as or similar contour parameter(s) to the target subject. By using such a customizable target posture model, the efficiency and/or accuracy of the target subject positioning may be improved. For example, the target posture model may be compared with a target subject model representing the target subject holding a posture to determine whether the posture of the target subject needs to be adjusted. As another example, the target posture model and the target subject model may be displayed jointly in a composite image, which may be used to guide the target subject to adjust his/her posture. Compared to a conventional way that a user needs to manually check and/or guide the positioning of the target subject, the automated subject positioning systems and methods disclosed herein may be more accurate and efficient by, e.g., reducing the workload of a user, cross-user variations, and the time needed for the subject positioning.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, the process 1700 may further include an operation to update the target subject model based on new target image data of the target subject captured after the posture of the target subject is adjusted. As another example, the process 1700 may further include an operation to determine whether the posture of the target subject needs to be further adjusted based on the updated target subject model and the target posture model.

FIG. 18 is a schematic diagram illustrating an exemplary composite image 1800 according to some embodiments of the present disclosure. As shown in FIG. 18, the composite image 1800 may include a representation 1810 of a target subject and a representation 1820 of the target posture model. The representation 1820 of the target posture model is superimposed on the representation 1810 of the target subject.

Merely for illustration purposes, the representation 1810 of the target subject in FIG. 18 is presented in the form of a 2D model. The 2D model of the target subject may be generated based on target image data of the target subject captured by an image capturing device after the target subject is positioned at the scan position. For example, the 2D model of the target subject may illustrate a posture (e.g., a contour) of the target subject in 2D space.

In some embodiments, the processing device 120 may determine whether the posture of the target subject needs to be adjusted based on the composite image 1800. For example, a matching degree between the target subject model and the target posture model may be based on the composted image 1800. As another example, the processing device 120 may transmit the composite image 1800 to a terminal device of a user for display. The user may view the composite image 1800 and determine whether the posture of the target subject needs to be adjusted based on the composite image 1800. Additionally or alternatively, the processing device 120 may transmit the composite image 1800 to a terminal device of the target subject to guide the target subject to adjust his/her posture.

It should be noted that the example illustrated in FIG. 18 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For instance, the representation 1810 of the target subject may be presented in the form of a 3D mesh model, a 3D skeleton model, a real image of the target subject, etc. As another example, the representation 1820 of the target posture model may be in the form of a 2D skeleton model.

FIG. 19 is a flowchart illustrating an exemplary process for image display according to some embodiments of the present disclosure. In some embodiments, the process 1900 may be implemented in the imaging system 100 illustrated in FIG. 1. For example, the process 1900 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 1900 may be accomplished with one or more additional operations not described, and/or without one or more of the operations discussed. Additionally, the order in which the operations of the process 1900 as illustrated in FIG. 19 and described below is not intended to be limiting.

In 1910, the processing device 120 (e.g., the acquisition module 710) may obtain image data of a target subject scanned or to be scanned by a medical imaging device.

The image data of the target subject may include image data corresponding to the entire target subject or image data corresponding to a portion of the target subject. In some embodiments, the medical imaging device (e.g., the medical imaging device 110) may be a suspended X-ray medical imaging device, a digital radiography (DR) device (e.g., a mobile digital X-ray medical imaging device), a C-arm device, a CT device, a PET device, an MRI device, or the like, as described elsewhere in the present disclosure.

In some embodiments, the image data may include first image data captured by a third image capturing device (e.g., the image capturing device 160) before the target subject is positioned at a scan position for receiving the scan. For example, the third imaging capturing device may obtain the first image data when or after the target subject enters the examination room. The first image data may be used to generate a target posture model of the target subject. Additionally or alternatively, the first image data may be used to determine one or more scanning parameters relating to a scan to be performed on the target subject by the medical imaging device. For instance, the one or more scanning parameters may include a target position of each of one or more movable components of the medical imaging device, such as a scanning table (e.g., the scanning table 114), a detector (e.g., the detector 112, the flat panel detector 440), an X-ray source (e.g., a tube, the radiation source 115, the X-ray source 420), or the like, or any combination thereof. As another example, the one or more scanning parameters may include one or more parameters relating to a light field of the medical imaging device, such as a target size of the light field.

In some embodiments, the image data may include second image data (or referred to as target image data) captured by a fourth image capturing device (e.g., the image capturing device 160) after the target subject is positioned at the scan position for receiving the scan. The third and fourth image capturing devices may be the same or different. For example, the target subject may hold a posture after he/she is positioned at the scan position, and the second image data may be used to generate a representation of the target subject holding the posture (such as, a target subject model). As another example, the scan may include a first scan to be performed on a first ROI of the target subject and a second scan to be performed on a second ROI of the target subject. The processing device may identify a first region corresponding to the first ROI and a second region corresponding to the second ROI based on the second image data.

In some embodiments, the image data may include third image data. The third image data may include a first image of the target subject captured using a fifth image capturing device or a medical imaging device (e.g., the medical imaging device 110). The fifth image capturing device may be the same as or different from the third or fourth image capturing devices. For example, the first image may be captured by a camera after the target subject is positioned at a scan position. As another example, the first image may be generated based on medical image data acquired by an X-ray imaging device in an X-ray scan of the target subject. The processing device 120 may process the first image to determine an orientation of the target subject.

In 1920, the processing device 120 (e.g., the analyzing module 720) may generate a display image based on the image data.

In some embodiments, the display image may include a first display image that is a composite image (e.g., the composite image 1800 as shown in FIG. 18) illustrating the target subject and a target posture model of the target subject. In the first display image, a representation of the target posture model may be superimposed on the representation of the target subject. For instance, the representation of the target subject may be a real human or a target subject model representing the target subject. In some embodiments, the image data obtained in 1910 may include the second image data as aforementioned. The processing device 120 may generate the first display image based on the second image data and the target posture model. The processing device 120 may further determine whether the posture of the target subject needs to be adjusted based on the first display image. More descriptions regarding the generation of the first display image and the determination of whether the posture of the target subject needs to be adjusted may be found elsewhere in the present disclosure, for example, in FIG. 17 and the descriptions thereof.

In some embodiments, the display image may include a second display image. The second display image may be an image illustrating position(s) of one or more components of the medical imaging device relative to the target subject. For example, the medical imaging device may include a plurality of ionization chambers. The second display image may include a first target image indicating a position of each of one or more candidate ionization chambers relative to an ROI of the target subject. The one or more candidate ionization chambers may be selected from the plurality of ionization chambers of the medical imaging device. As another example, the second display image may include a second target image indicating position(s) of at least some of the plurality of ionization chambers relative to the ROI of the target subject. The first target image and/or the second target image may be used to select one or more target ionization chambers among the plurality of ionization chambers, wherein the target ionization chamber(s) may be actuated in a scan of the ROI of the target subject. More descriptions regarding the first target image and/or the second target image may be found elsewhere in the present disclosure, for example, in FIGS. 16A-16C and the description thereof.

As yet another example, the second display image may include a third target image indicating target position(s) of the one or more movable components (e.g., a detector, a radiation source) of the medical imaging device relative to the target subject. The third target image may be used to determine whether the target position(s) of the one or more movable components of the medical imaging device needs to be adjusted. For instance, the target position(s) of the one or more movable components may be determined by performing operations 1010-1030.

In some embodiments, the display image may include a third display image illustrating a position of a light field of the medical imaging device relative to the target subject. For example, the processing device 120 may obtain one or more parameters of the light field, and generate the third display image based on the one or more parameters of the light field and the image data acquired in operation 1910. For instance, the one or more parameters of the light field may include a position, a target size, a width, a height, or the like, of the light field. Merely by way of example, in the third display image, a region corresponding to the light field may be marked on a representation of the target subject. The third display image may be used to determine whether the one or more parameters of the light field of the medical imaging device needs to be adjusted. Additionally or alternatively, the third display image may be used to determine whether the posture of the target subject needs to be adjusted. For example, to determine the one or more parameters of the light field, the processing device 120 may perform one or more operations that are similar to operations 1210-1220 as described in connection with FIG. 12.

In some embodiments, the display image may include a fourth display image in which a representation of the target subject has a reference orientation (e.g., a “head-up” orientation). For instance, the processing device 120 may determine an orientation of the target subject based on the image data of the target subject. The processing device 120 may further generate the fourth display image based on the orientation of the target subject and the image data of the target subject. In some embodiments, the processing device 120 may determine the orientation of the target subject based on the image data in a similar manner as how the orientation of the target subject is determined based on a first image as described in connection with FIG. 13. For example, the processing device may determine the orientation of the target subject based on an orientation of a target region corresponding to an ROI of the target subject in the image data. Alternatively, the processing device 120 may determine the orientation of the target subject based on a position of a target region corresponding to an ROI of the target subject in the image data.

It should be noted that the above mentioned features of the first, second, third, and fourth display images are provided for illustration purposes, and not intended to be limiting. In some embodiments, the display image may have a combination of features of two or more of the first display image, the second display image, the third display image, and the fourth display image. For instance, the display image (e.g., a display image 2000 as shown in FIG. 20) may indicate target position(s) of the one or more movable components of the medical imaging device relative to the target subject, the position(s) of one or more ionization chambers relative to the target subject, and also the position of light field relative to the target subject.

In 1930, the processing device 120 (e.g., the analyzing module 720) may transmit the display image to a terminal device for display.

In some embodiments, the terminal device may include a first terminal device of a user (e.g., a doctor, an operator). The user may view the display image via the first terminal device. In some embodiments, the display image may help the user to perform an analysis and/or a determination. For example, the user may view the first display image via the first terminal device and determine whether the posture of the target subject needs to be adjusted. Alternatively, the processing device 120 may determine whether the posture of the target subject needs to be adjusted based on the first display image. The user may view the first display image and confirm a determination result of whether the posture of the target subject needs to be adjusted. As another example, the user may view the second display image and determine whether the target position(s) of the one or more movable components of the target subject needs to be adjusted. Normally, for a medical imaging device including a scanning table, the detector is located underneath the scanning table, which makes it difficult to directly observe the position of the detector. The second display image may help the user to know the position of the detector in a more intuitive way, thereby improving the accuracy of the target position of the detector. As yet another example, the user may view the third display image and determine whether the one or more parameters relating to the light field needs to be adjusted. The user may adjust one or more parameters of the light field, such as the size and/or the position of the light field via the first terminal device (e.g., by moving the position of a representation of the light field in the third display image). As still another example, the user may view the fourth display image in which the representation of the target subject has the reference orientation. The fourth display image (e.g., a CT image, a PET image, an MRI image) may include anatomical information related to an ROI of the target subject and/or metabolic information related to the ROI. The user may make a diagnostic analysis based on the fourth display image.

Additionally or alternatively, the terminal device may include a second terminal device in the vicinity of the target subject. For instance, the second terminal device may be a display device mounted on the medical imaging device or the ceiling of the examination room). The second terminal device may display the first display image to the target subject. In some embodiments, an instruction may be provided to the target subject to guide the target subject to move one or more body parts of the target subject to hold a target posture. The instruction may be provided to the target subject via the second terminal device in the form of text, voice, an image, a video, a haptic alert, or the like, or any combination thereof. More information regarding the instruction for guiding the target subject may be found elsewhere in the present disclosure, for example, in operation 1930 and the description thereof.

In some embodiments, the terminal device may display the display image along with one or more interactive elements. The one or more interactive elements may be used to implement one or more interactions between the user (or the target subject) and the terminal device. For example, the interactive elements may include one or more keys, buttons, and/or input boxes for the user to make an adjustment to or confirm an analysis result generated by the processing device 120. As another example, the one or more interactive elements may include one or more image display options for the user to manipulate (e.g., zoom in, zoom out, add or modify an annotation) the display image. Merely by way of example, the user may manually adjust the one or more parameters of the light field in the third image by adjusting the contour of a representation of the light field in the third image, such as by dragging one or more lines of the contour of the representation of the light field using a mouse or a touchscreen.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For instance, at least one of the first display image, the second display image, the third display image, or the fourth display image may be transmitted to a storage device (e.g., the storage device 130) for storage.

FIG. 20 is a schematic diagram of an exemplary display image 2000 relating to a target subject according to some embodiments of the present disclosure. The chest of the target subject may be scanned by a medical imaging device. As shown in FIG. 20, the display image 2000 may include a representation 2010 of the target subject, a representation 2020 of a detector (such as the flat panel detector 440) of the medical imaging device, a plurality of representations 2030 of a plurality of ionization chambers of the medical imaging device, and a representation 2040 of a light field of the medical imaging device.

In some embodiments, the display image 2000 may be used to determine whether one or more parameters of the target subject and/or the medical imaging device need to be adjusted. Merely by way of example, as shown in FIG. 20, the representation 2040 of the light field covers a target region corresponding to the ROI (e.g., including the chest, not illustrated in FIG. 20) of the target subject, which suggests that the target size of the light field is suitable for the scan and does not need to be adjusted. The representation 2020 of the detector covers the representation 2040 of the light field in FIG. 20, which suggests that the position of the detector does not need to be adjusted.

In some embodiments, the display image 2000 may be used to select one or more target ionization chambers among the plurality of ionization chambers. As shown in FIG. 20, four ionization chambers are presented. The representations of three of the ionization chambers are covered by the target region, and a representation of one of the ionization chambers is not covered by the target region. In some embodiments, the processing device 120 may select the target ionization chamber(s) from the plurality of ionization chambers based on the display image 2000. For instance, the processing device 120 may select an ionization chamber that is closest to central point of the ROI of the target subject as a candidate ionization chamber. The processing device 120 may further determine whether a representation of the candidate ionization chamber is covered by the target region corresponding to the ROI in the display image 2000. In response to determining that the representation of the candidate ionization chamber is covered by the target region, the processing device 120 may determine that a position offset between the candidate ionization chamber and the ROI is negligible. The processing device 120 may further designate the candidate ionization chamber as a target ionization chamber corresponding to the ROI of the target subject.

Alternatively, the processing device 120 may add an annotation indicating the candidate ionization chamber in the display image 2000 and/or mark the representation of the candidate ionization chamber using a color that is different from other ionization chambers in the display image 2000. The display image 2000 may be displayed to a user via a display (e.g., the display 320 of the mobile device 300). The user may determine whether the candidate ionization chamber should be designated as one of the target ionization chamber(s). In some embodiments, the three ionization chambers whose the representations in the display image 2000 are covered by the target region corresponding to the ROI may be selected as candidate ionization chambers. The user may provide a user input indicating the target ionization chamber(s) selected from the candidate ionization chambers.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For instance, the display image 2000 may further include other information relating to the target subject, such as an imaging protocol of the scan.

FIG. 21 is a flowchart illustrating an exemplary process for imaging a target subject according to some embodiments of the present disclosure. In some embodiments, the process 2100 may be implemented in the imaging system 100 illustrated in FIG. 1. For example, the process 2100 may be stored in a storage device (e.g., the storage device 130, the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 7). The operations of the illustrated process presented below are intended to be illustrative. In some embodiments, the process 2100 may be accomplished with one or more additional operations not described, and/or without one or more of the operations discussed. Additionally, the order in which the operations of the process 2100 as illustrated in FIG. 21 and described below is not intended to be limiting.

In some embodiments, the process 2100 may be implemented in a scan on an ROI of the target subject. In some embodiments, the ROI may include a lower limb of the target subject or a portion of the lower limb. For instance, the lower limb may include a foot, an ankle, a leg (e.g., a calf and/or a thigh), a pelvis, or the like, or any combination thereof.

In some embodiments, the process 2100 may be implemented in a stitching scan of the target subject. In a stitching scan of the target subject, a plurality of scans may be performed on a plurality of ROIs of the target subject in sequence to acquire a stitched image of the ROIs. For illustration purposes, the following descriptions are described with reference to a stitching scan performed on a first ROI and a second ROI of the target subject, and not intended to limit the scope of the present disclosure. The first and second ROIs may be two different regions partially overlap with each other or not overlap at all. The first ROI may be scanned before the second ROI in the stitching scan. Merely by way of example, the first ROI may be the chest of the target subject, and the second ROI may be a lower limb of the target subject (or a portion of the lower limb). A stitched image corresponding to the chest and the lower limb of the target subject may be generated by the stitching scan.

In 2110, the processing device 120 (e.g., the control module 730) may cause a supporting device to move from an initial device position to a target device position.

In some embodiments, the supporting device may include a supporting component (e.g., supporting component 451), a first driving component (e.g., the first driving component 452), a second driving component (e.g., the second driving component 453), a fixing component (e.g., the fixing component 454), a handle (e.g., the handle 456), and a panel (e.g., the panel 455) as described elsewhere in the present disclosure (e.g., FIGS. 4A-4B, and descriptions thereof). In some embodiments, before a scan (e.g., a first scan of a stitching scan) is performed on the target subject, the processing device 120 may control the first driving component to cause the supporting device to move from an initial device position to a target device position. The initial device position refers to an initial position where the supporting device is located before the scan of the target subject. For example, the supporting device may be stored and/or charged at a preset position in the examination room when it is not in use, and the preset position may be regarded as the initial device position. The target device position refers to a position where the supporting device is located during the scan of the target subject. For example, the supporting device may be located in the vicinity of the medical imaging device, such as, at a certain distance (e.g., 5 centimeters, 10 centimeters) in front of a detector (e.g., the flat panel detector 440) of the medical imaging device during the scan as shown in FIG. 4B. In some embodiments, the supporting device may be fixed at the initial device position and/or the target device position by the fixing component.

In 2120, the processing device 120 (e.g., the control module 730) may cause the supporting device to move a target subject from an initial subject position to a target subject position (or referred to as a first position).

In some embodiments, before the first scan, the target subject may be moved to the target subject position so that the first ROI may be located at a suitable position for receiving the first scan. For example, when the target subject is located at the target subject position, during the first scan, a radiation source of the medical imaging device may emit a radiation beam towards the first ROI and a detector (e.g., the flat panel detector 440) of the medical imaging device may cover the entire first ROI of the target subject. In some embodiments, after the first scan, the detector may be moved to another position so that the detector can cover the entire second ROI of the target subject during the second scan. Similarly, the radiation source may be moved to another position so that the radiation source may emit a radiation beam towards the second ROI during the second scan. The target subject may be supported at the target subject position during the first scan and the second scan. In some embodiments, the processing device 120 may determine the target subject position based on the first region, the second region, a moving range of the detector, a moving range of the radiation source, the height of the target subject, or the like, or any combination thereof.

In some embodiments, the target subject position may be represented as a coordinate of a physical point (e.g., on the feet, the head, or the first ROI) of the target subject in a coordinate system. Merely by way of example, the target subject position may be presented as a Z-axis coordinate of the feet of the target subject in the coordinate system 470 as shown in FIG. 4A. The target subject position may be set manually by a user (e.g., a doctor, an operator of the medical imaging device, or the like). For instance, the user may manually input information regarding the target subject position (e.g., a value of a vertical distance between the target subject position and the floor of the examination room) via a terminal device. The supporting device may receive the information regarding the target subject position and set the target subject position based on the information regarding the target subject position. As another example, the user may set the target subject position by manually controlling the movement of the supporting device (e.g., using one or more buttons on the supporting device and/or the terminal device). Alternatively, the processing device 120 may determine the target subject position based on image data of the target subject.

For example, the processing device 120 may obtain the image data of the target subject from an image capturing device mounted in the examination room. The processing device 120 may then generate a subject model representing the target subject based on the image data of the target subject, and identify a first region corresponding to the first ROI from the subject model. More descriptions of the identification of a region corresponding to an ROI from a subject model may be found elsewhere in the present disclosure (e.g., operation 1020 in process 1000 and descriptions thereof). Alternatively, the processing device 120 may identify the first region from the original image data or a target posture model of the target subject.

In some embodiments, after the supporting device is moved to the target device position, the processing device 120 may cause a first notification to be generated, wherein the first notification may be used to notify the target subject to step on the supporting device before the first scan. The first notification may be in the form of text, voice, an image, a video, a haptic alert, or the like, or any combination thereof. The first notification may be outputted by, for example, a terminal device in the vicinity of the target subject, the supporting device, or the medical imaging device. For example, the processing device 120 may cause the supporting device to output a voice notification of “please step on the supporting device.”

In some embodiments, before the first scan and after the target subject steps on the supporting device, the processing device 120 may control the second driving component to cause the supporting device to move the target subject from an initial subject position to the target subject position along a target direction. The initial subject position refers to a position of the target subject after the target subject steps on the supporting device. For example, the target direction may be the Z-axis direction of the coordinate system 470 as shown in FIG. 4A. The second driving component may include a lifting mechanism that may lift up the target subject so as to move the target subject from the initial subject position to the target subject position.

Additionally or alternatively, before or after the target subject steps on the supporting device, a position of the handle of the supporting device may be adjusted so that the target subject can put his/her hands on the handle when the target subject is supported by the supporting device. The position of the handle may be set manually by a user (e.g., a doctor, an operator of the medical imaging device, or the like). For instance, the user may manually input information regarding the position of the handle (e.g., a value of a vertical distance between the handle and the ground of the examination room) via a terminal device. The supporting device may receive the information regarding the handle and set the position of the handle based on the information regarding the position of the handle. As another example, the user may set the position of the handle by manually controlling the movement of the handle (e.g., using one or more buttons on the supporting device and/or the terminal device). Alternatively, the processing device 120 may determine the position of the handle based on the image data of the target subject, a scan position (e.g., the target subject position) of the target subject, or the like. For example, the processing device 120 may determine a distance of the handle to the supporting component of the supporting device as ⅔ of the height of the target subject.

In 2130, the processing device 120 (e.g., the control module 730) may cause the medical imaging device to perform the first scan on the first ROI of the target subject. The target subject may hold an upright posture.

The upright posture may include a standing posture, a sitting posture, a kneeling posture, or the like. The target subject may be supported by a supporting device (e.g., the supporting device 460) at a target subject position during the first scan. For example, the target subject may stand, sit, or kneel on the supporting device to receive the first scan. In some embodiments, the medical imaging device (e.g., the medical imaging device 110) may be an X-ray imaging device (e.g., a suspended X-ray imaging device, a C-arm X-ray imaging device), a digital radiography (DR) device (e.g., a mobile digital X-ray imaging device), a CT device, or the like, as described elsewhere in the present disclosure.

In some embodiments, the processing device 120 may obtain one or more first scanning parameters related to the first scan and perform the first scan on the first ROI of the target subject according to the one or more first scanning parameters. For example, the one or more first scanning parameters may include a scanning angle, a position of a radiation source, a position of a scanning table, an inclination angle of the scanning table, a position of a detector, a gantry angle of a gantry, a size of a field of view (FOV), a shape of a collimator, a current of the radiation source, a voltage of the radiation source, or the like, or any combination thereof.

In some embodiments, the processing device 120 may obtain a parameter value of a scanning parameter based on an imaging protocol relating to the first scan to be performed on the target subject. For example, the protocol may be predetermined and stored in a storage (e.g., the storage device 130). As another example, at least a portion of the protocol may be determined manually by a user (e.g., an operator). In some embodiments, the processing device 120 may determine a parameter value of a scan parameter based on image data relating to the examination room acquired by an image capturing device mounted in the examination room. For example, the image data may illustrate a radiation source and/or a detector of the medical imaging device. The processing device 120 may determine the position of the radiation source and/or the detector based on the image data.

In 2140, the processing device 120 (e.g., the control module 730) may cause the medical imaging device to perform the second scan on the second ROI of the target subject.

In some embodiments, after the first scan and before the second scan, the radiation source and/or the detector may be moved to the suitable position(s) for performing the second scan on the second ROI. The suitable position(s) of the radiation source and/or the detector may be determined based on image data of the target subject captured in operation 1010. More descriptions regarding determining a suitable position for a movable component of a medical imaging device for performing a scan on a target subject may be found elsewhere in the present disclosure, for example, in operation 1020 of FIG. 10 and the descriptions thereof.

In some embodiments, after the first scan and before the second scan, the processing device 120 may control the second driving component to cause the supporting device to move the target subject from a first position (e.g., the target subject position during the first scan) to a second position. Additionally or alternatively, when the supporting device moves the target subject from the first position to the second position, one or more movable components (e.g., a detector) of the medical imaging device may also move along, for example, the target direction or an opposite direction of the target direction. For instance, when the supporting device moves the target subject upward from the target subject position to the second position, the detector, such as the flat panel detector 440, may move downward to a suitable position.

In some embodiments, after the second scan, the processing device 120 may cause a second notification to be generated, wherein the second notification may be used to notify the target subject to step off from the supporting device. The second notification may be in the form of text, voice, an image, a video, a haptic alert, or the like, or any combination thereof. The form of the second notification may be the same as or different from the form of the first notification. The second notification may be outputted by, for example, a terminal device in the vicinity of the target subject, the supporting device, or the medical imaging device. For example, the processing device 120 may cause the supporting device to output a voice notification of “please step off the supporting device.”

In some embodiments, after the second scan, the processing device 120 may control the first driving component to cause the supporting device to move from the target device position back to the initial device position. For example, after the target subject steps off the supporting device, the processing device 120 may control the first driving component to cause the supporting device to move from the target device position back to the initial device position for charge.

In 2150, the processing device 120 (e.g., the acquisition module 710) may obtain first scan data relating to the first scan and second scan data relating to the second scan.

The first scan data and the second scan data (also referred to as medical image data) may include projection data, one or more images generated based on the projection data, or the like. In some embodiments, the processing device 120 may obtain the first scan data and the second scan data from the medical imaging device. Alternatively, the first scan data and the second scan data may be acquired by the medical imaging device and stored in a storage device (e.g., the storage device 130, the storage device 220, the storage 390, or an external source). The processing device 120 may retrieve the first scan data and the second scan data from the storage device.

In 2160, the processing device 120 (e.g., the analyzing module 720) may generate an image corresponding to the first ROI and the second ROI of the target subject.

In some embodiments, the processing device 120 may generate an image A corresponding to the first ROI based on the first scan data and an image B corresponding to the second ROI based on the second scan data. The processing device 120 may further generate the image corresponding to the first and second ROIs based on the image A and the image B. For example, the processing device 120 may generate the image corresponding to the first ROI and the second ROI by stitching the images A and B according to one or more image stitching algorithms. Exemplary image stitching algorithms may include a normalized cross correlation-based image stitching algorithm, a mutual information based image stitching algorithm, a low-level feature-based image stitching algorithm (e.g., a Harris corner detector-based image stitching algorithm, a fast corner detector-based image stitching algorithm, a sift feature detector-based image stitching algorithm, a surf feature detector based image stitching algorithm), a contour-based image stitching algorithm, or the like.

It should be noted that the above description regarding the process 2100 is provided for the purposes of illustration, and not intended to limit the scope of the present disclosure. In some embodiments, more than two ROIs of the target subject may be scanned according to a specific sequence in the stitching scan. Each pair of ROIs that are adjacent in the specific sequence may include an ROI scanned at a first time point and an ROI scanned at a second time point after the first time point. The ROI scanned at the first time point may be regarded as a first ROI, and the ROI scanned at the second time point may be regarded as a second ROI. The processing device 120 may perform the process 2100 (or a portion thereof) for each pair of ROIs that are adjacent in the specific sequence. In some embodiments, one or more of additional scans (e.g., a third scan, a fourth scan) may be performed on one or more other ROIs (e.g., a third ROI, a fourth ROI) of the target subject. A stitched image corresponding to the first ROI, the second ROI, and the other ROI(s) may be generated.

Compared with conventional stitching imaging procedures which need a user (e.g., a doctor) to determine a plurality of scan positions (e.g., the first position, the second position) of the target subject, a stitching imaging procedure (e.g., the process 2100) disclosed in the present disclosure may be implemented with reduced or minimal or without user intervention, which is time-saving, more efficient, and more accurate. For example, the scan positions of the target subject may be determined by analyzing image data of the target subject instead of by a user manually. In addition, the stitching imaging procedure disclosed herein may utilize a supporting device to achieve an automated positioning of the target subject, for example, by moving the target subject to the target subject position and/or the second position automatically. In this way, the determined scan position(s) may be more accurate, and the positioning of the target subject to the scan position(s) may be implemented more precisely, which in turn, may improve the efficiency and/or accuracy of the stitching scan of the target subject. Furthermore, the position of the handle may be determined and adjusted automatically based on the scan position of the target subject and/or the height of the target subject, which may be convenient for the target subject to get on and/or get off the supporting device.

In some embodiments, one or more operations may be added or omitted. For example, an operation for determining the target subject position of the target subject may be added before operation 2120. As another example, the scan to be performed on the target subject may be a non-stitching scan. In operation 2130, the processing device 120 (e.g., the control module 730) may perform a single scan on the first ROI of the target subject when the target subject is supported by the supporting device at the target subject position. An image may be generated based on scan data acquired during the scan. Operations 2140-2160 may be omitted. In some embodiments, two or more operations of the process 2100 may be performed simultaneously or in any suitable order.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer-readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in a combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby, and Groovy, or other programming languages. The program code may execute entirely on the users computer, partly on the users computer, as a stand-alone software package, partly on the users computer and partly on a remote computer or entirely on the remote computer or server. In the latter scenario, the remote computer may be connected to the users computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

1. A method for subject identification implemented on a computing device having one or more processors and one or more storage devices, the method comprising:
obtaining image data of at least one candidate subject, the image data being captured by an image capturing device when or after the at least one candidate subject enters an examination room;
obtaining reference information associated with a target subject to be examined; and
identifying, from the at least one candidate subject, the target subject based on the reference information and the image data.

2. The method of claim 1, wherein the obtaining reference information associated with a target subject to be examined comprises:
obtaining a replication image of at least one of an identity card, a medical insurance card, a medical card, or an examination application form of the target subject; and
determining, based on the replication image, the reference information.

3. The method of claim 2, wherein the reference information associated with the target subject includes reference image data of the target subject.

4. The method of claim 3, wherein the obtaining reference information associated with a target subject to be examined comprises:
obtaining the reference image data of the target subject captured by a second image capturing device before the target subject enters the examination room.

5. The method of claim 3, wherein the identifying, from the at least one candidate subject, the target subject based on the reference Information and the image data comprises:
extracting, from the reference image data, reference feature information of the target subject;
extracting, from the image data, feature information of each of the at least one candidate subject; and
identifying, based on the reference feature information of the target subject and the feature information of the each of the at least one candidate subject, the target subject.

6. The method of claim 2, wherein the reference information associated with the target subject includes reference identity information of the target subject.

7. The method of claim 6, wherein the identifying, from the at least one candidate subject, the target subject based on the reference Information and the image data comprises:
determining, based on the image data, identity information of each of the at least one candidate subject; and
identifying, from the at least one candidate subject, the target subject by comparing the identity information of the each of the at least one candidate subject with the reference identity information of the target subject.

8. The method of claim 6, wherein the determining, based on the image data, identity information of each of the at least one candidate subject comprises:
for the each of the at least one candidate subject, segmenting, from the image data, a human face of the candidate subject; and
determining, based on the human face of the candidate subject and from an identity information database, the identity information of the candidate subject.

9. The method of claim 1, wherein
the obtaining reference information associated with a target subject to be examined comprises:
causing a terminal device of a user to display the image data of the at least one candidate subject; and
obtaining, via the terminal device, an input associated with the target subject from the user, and
the identifying, from the at least one candidate subject, the target subject based on the reference information and the image data comprises:
identifying, from the at least one candidate subject, the target subject based on the input.

10. The method of claim 1, further comprising:
generating a subject model of the target subject based on the image data;
obtaining a reference posture model associated with the target subject; and
generating a target posture model of the target subject based on the subject model and the reference posture model.

11-18. (canceled)

19. A method for scan preparation implemented on a computing device having one or more processors and one or more storage devices, the method The method of claim 1, further comprising:
for one or more movable components of a medical imaging device, determining, based on the image data, a target position of each of the one or more movable components;
for each of the one or more movable components of the medical imaging device, causing the movable component to move to the target position of the movable component; and
causing the medical imaging device to scan the target subject when the each of the one or more movable components of the medical imaging device is at its respective target position.

20-25. (canceled)

26. The method of claim 1, further comprising:
determining, based on the image data, one or more parameter values of a light field; and
causing a medical imaging device to scan the target subject according to the one or more parameter values of the light field.

27-33. (canceled)

34. The method of claim 1, further comprising:
obtaining a first image of the target subject;
determining, based on the first image, an orientation of the target subject; and
causing a terminal device to display a second image of the target subject based on the first image and the orientation of the target subject, wherein a representation of the target subject having a reference orientation in the second image.

35-43. (canceled)

44. The method of claim 1, further comprising:
obtaining at least one parameter value of at least one scanning parameter relating to a scan to be performed on the target subject;
obtaining a relationship between a reference dose and the at least one scanning parameter; and
determining, based on the relationship and the at least one parameter value of the at least one scanning parameter, a value of an estimated dose associated with the target subject.

45-53. (canceled)

54. The method of claim 1, further comprising:
obtaining target image data of the target subject to be scanned by a medical imaging device, the medical imaging device including a plurality of ionization chambers;
selecting, among the plurality of ionization chambers, at least one target ionization chamber based on the target image data; and
causing the medical imaging device to scan the target subject using the at least one target ionization chamber.

55-63. (canceled)

64. The method of claim 1, further comprising:
obtaining target image data of the target subject holding a posture captured by the image capturing device;
obtaining a target posture model representing a target posture of the target subject; and
determining, based on the target image data and the target posture model, whether the posture of the target subject needs to be adjusted.

65-76. (canceled)

77. The method of claim 1, further comprising:
obtaining target image data of the target subject scanned or to be scanned by a medical imaging device;
generating a display image based on the target image data; and
transmitting the display image to the terminal device for display.

78-86. (canceled)

87. The method of claim 1, further comprising:
causing a supporting device to move the target subject from an initial subject position to a target subject position;
causing a medical imaging device to perform a scan on a region of interest (ROI) of the target subject, the target subject holding an upright posture and being supported by the supporting device at the target subject position during the scan;
obtaining scan data relating to the scan; and
generating an image corresponding to the ROI based on the scan data.

88-98. (canceled)

99. The method of claim 1, further comprising:
obtaining an image of the target subject;
determining, based on the image, an orientation of the target subject;
adjusting the image based on the orientation of the target subject; and
causing a terminal device to display an adjusted image of the target subject, wherein a representation of the target subject having a reference orientation in the adjusted image.

100-105. (canceled)

106. A system for subject identification, comprising:
at least one storage device including a set of instructions; and
at least one processor configured to communicate with the at least one storage device, wherein when executing the set of instructions, the at least one processor is configured to direct the system to perform operations including:
obtaining image data of at least one candidate subject, the image data being captured by an image capturing device when or after the at least one candidate subject enters an examination room;
obtaining reference information associated with a target subject to be examined; and
identifying, from the at least one candidate subject, the target subject based on the reference information and the image data.
